SEQUENCE LISTING (29 sequences)

SEQ ID NO: 1 - DNA, 1494 bp, Homo sapiens
atgcccacccccgacgccaccacgccacaggccaagggcttccgcagggccgtgtctgagctggacgccaagcaggcagaggccatcatgtccccgcggttcattgggcgcaggcagagcctcatcgaggacgcccgcaaggagcgggaggcggcggtggcagcagcggccgctgcagtcccctcggagcccggggaccccctggaggctgtggcctttgaggagaaggaggggaaggccgtgctaaacctgctcttctccccgagggccaccaagccctcggcgctgtcccgagctgtgaaggtgtttgagacgtttgaagccaaaatccaccatctagagacccggcccgcccagaggccgcgagctgggggcccccacctggagtacttcgtgcgcctcgaggtgcgccgaggggacctggccgccctgctcagtggtgtgcgccaggtgtcagaggacgtgcgcagccccgcggggcccaaggtcccctggttcccaagaaaagtgtcagagctggacaagtgtcatcacctggtcaccaagttcgaccctgacctggacttggaccacccgggcttctcggaccaggtgtaccgccagcgcaggaagctgattgctgagatcgccttccagtacaggcacggcgacccgattccccgtgtggagtacaccgccgaggagattgccacctggaaggaggtctacaccacgctgaagggcctctacgccacgcacgcctgcggggagcacctggaggcctttgctttgctggagcgcttcagcggctaccgggaagacaatatcccccagctggaggacgtctcccgcttcctgaaggagcgcacgggcttccagctgcggcctgtggccggcctgctgtccgcccgggacttcctggccagcctggccttccgcgtgttccagtgcacccagtatatccgccacgcgtcctcgcccatgcactcccctgagccggactgctgccacgagctgctggggcacgtgcccatgctggccgaccgcaccttcgcgcagttctcgcaggacattggcctggcgtccctgggggcctcggatgaggaaattgagaagctgtccacgctgtactggttcacggtggagttcgggctgtgtaagcagaacggggaggtgaaggcctatggtgccgggctgctgtcctcctacggggagctcctgcactgcctgtctgaggagcctgagattcgggccttcgaccctgaggctgcggccgtgcagccctaccaagaccagacgtaccagtcagtctacttcgtgtctgagagcttcagtgacgccaaggacaagctcaggagctatgcctcacgcatccagcgccccttctccgtgaagttcgacccgtacacgctggccatcgacgtgctggacagcccccaggccgtgcggcgctccctggagggtgtccaggatgagctggacacccttgcccatgcgctgagtgccattggctag

SEQ ID NO: 2 - protein, 497 aa, Homo sapiens
MPTPDATTPQAKGFRRAVSELDAKQAEAIMSPRFIGRRQSLIEDARKEREAAVAAAAAAVPSEPGDPLEAVAFEEKEGKAVLNLLFSPRATKPSALSRAVKVFETFEAKIHHLETRPAQRPRAGGPHLEYFVRLEVRRGDLAALLSGVRQVSEDVRSPAGPKVPWFPRKVSELDKCHHLVTKFDPDLDLDHPGFSDQVYRQRRKLIAEIAFQYRHGDPIPRVEYTAEEIATWKEVYTTLKGLYATHACGEHLEAFALLERFSGYREDNIPQLEDVSRFLKERTGFQLRPVAGLLSARDFLASLAFRVFQCTQYIRHASSPMHSPEPDCCHELLGHVPMLADRTFAQFSQDIGLASLGASDEEIEKLSTLYWFTVEFGLCKQNGEVKAYGAGLLSSYGELLHCLSEEPEIRAFDPEAAAVQPYQDQTYQSVYFVSESFSDAKDKLRSYASRIQRPFSVKFDPYTLAIDVLDSPQAVRRSLEGVQDELDTLAHALSAIG

SEQ ID NO: 3 - DNA, 1029 bp, Homo sapiens
atgagccccgcggggcccaaggtcccctggttcccaagaaaagtgtcagagctggacaagtgtcatcacctggtcaccaagttcgaccctgacctggacttggaccacccgggcttctcggaccaggtgtaccgccagcgcaggaagctgattgctgagatcgccttccagtacaggcacggcgacccgattccccgtgtggagtacaccgccgaggagattgccacctggaaggaggtctacaccacgctgaagggcctctacgccacgcacgcctgcggggagcacctggaggcctttgctttgctggagcgcttcagcggctaccgggaagacaatatcccccagctggaggacgtctcccgcttcctgaaggagcgcacgggcttccagctgcggcctgtggccggcctgctgtccgcccgggacttcctggccagcctggccttccgcgtgttccagtgcacccagtatatccgccacgcgtcctcgcccatgcactcccctgagccggactgctgccacgagctgctggggcacgtgcccatgctggccgaccgcaccttcgcgcagttctcgcaggacattggcctggcgtccctgggggcctcggatgaggaaattgagaagctgtccacgctgtactggttcacggtggagttcgggctgtgtaagcagaacggggaggtgaaggcctatggtgccgggctgctgtcctcctacggggagctcctgcactgcctgtctgaggagcctgagattcgggccttcgaccctgaggctgcggccgtgcagccctaccaagaccagacgtaccagtcagtctacttcgtgtctgagagcttcagtgacgccaaggacaagctcaggagctatgcctcacgcatccagcgccccttctccgtgaagttcgacccgtacacgctggccatcgacgtgctggacagcccccaggccgtgcggcgctccctggagggtgtccaggatgagctggacacccttgcccatgcgctgagtgccattggctag

SEQ ID NO: 4 - protein, 342 aa, Homo sapiens
MSPAGPKVPWFPRKVSELDKCHHLVTKFDPDLDLDHPGFSDQVYRQRRKLIAEIAFQYRHGDPIPRVEYTAEEIATWKEVYTTLKGLYATHACGEHLEAFALLERFSGYREDNIPQLEDVSRFLKERTGFQLRPVAGLLSARDFLASLAFRVFQCTQYIRHASSPMHSPEPDCCHELLGHVPMLADRTFAQFSQDIGLASLGASDEEIEKLSTLYWFTVEFGLCKQNGEVKAYGAGLLSSYGELLHCLSEEPEIRAFDPEAAAVQPYQDQTYQSVYFVSESFSDAKDKLRSYASRIQRPFSVKFDPYTLAIDVLDSPQAVRRSLEGVQDELDTLAHALSAIG

SEQ ID NO: 5 - DNA, 792 bp, Mus musculus
ggtggttttcctttgaaaaacacgatgataatatggccacaaccgcggccgtagatcccgggaccatggagaagccgcggggagtcaggtgcaccaatgggttctccgagcgggagctgccgcggcccggggccagcccgcctgccgagaagtcccggccgcccgaggccaagggcgcacagccggccgacgcctggaaggcagggcggcaccgcagcgaggaggaaaaccaggtgaacctccccaaactggcggctgcttactcgtccattctgctctcgctgggcgaggacccccagcggcaggggctgctcaagacgccctggagggcggccaccgccatgcagtacttcaccaagggataccaggagaccatctcagatgtcctgaatgatgctatatttgatgaagatcatgacgagatggtgattgtgaaggacatagatatgttctccatgtgtgagcatcaccttgttccatttgtaggaagggtccatattggctatcttcctaacaagcaagtccttggtctcagtaaacttgccaggattgtagaaatctacagtagacgactacaagttcaagagcgcctcaccaaacagattgcggtggccatcacagaagccttgcagcctgctggcgttggagtagtgattgaagcgacacacatgtgcatggtaatgcgaggcgtgcagaaaatgaacagcaagactgtcactagcaccatgctgggcgtgttccgggaagaccccaagactcgggaggagttcctcacactaatcaggagctgag

SEQ ID NO: 6 - DNA, 753 bp, Homo sapiens
atggagaagggccctgtgcgggcaccggcggagaagccgcggggcgccaggtgcagcaatgggttccccgagcgggatccgccgcggcccgggcccagcaggccggcggagaagcccccgcggcccgaggccaagagcgcgcagcccgcggacggctggaagggcgagcggccccgcagcgaggaggataacgagctgaacctccctaacctggcagccgcctactcgtccatcctgagctcgctgggcgagaacccccagcggcaagggctgctcaagacgccctggagggcggcctcggccatgcagttcttcaccaagggctaccaggagaccatctcagatgtcctaaacgatgctatatttgatgaagatcatgatgagatggtgattgtgaaggacatagacatgttttccatgtgtgagcatcacttggttccatttgttggaaaggtccatattggttatcttcctaacaagcaagtccttggcctcagcaaacttgcgaggattgtagaaatctatagtagaagactacaagttcaggagcgccttacaaaacaaattgctgtagcaatcacggaagccttgcggcctgctggagtcggggtagtggttgaagcaacacacatgtgtatggtaatgcgaggtgtacagaaaatgaacagcaaaactgtgaccagcacaatgttgggtgtgttccgggaggatccaaagactcgggaagagttcctgactctcattaggagctga

SEQ ID NO: 7 - protein, 250 aa, Homo sapiens
MEKGPVRAPAEKPRGARCSNGFPERDPPRPGPSRPAEKPPRPEAKSAQPADGWKGERPRSEEDNELNLPNLAAAYSSILSSLGENPQRQGLLKTPWRAASAMQFFTKGYQETISDVLNDAIFDEDHDEMVIVKDIDMFSMCEHHLVPFVGKVHIGYLPNKQVLGLSKLARIVEIYSRRLQVQERLTKQIAVAITEALRPAGVGVVVEATHMCMVMRGVQKMNSKTVTSTMLGVFREDPKTREEFLTLIRS

SEQ ID NO: 8 - DNA, 798 bp, Artificial Sequence
  CBh promoter
cgttacataacttacggtaaatggcccgcctggctgaccgcccaacgacccccgcccattgacgtcaatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattgtgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagctgagcaagaggtaagggtttaagggatggttggttggtggggtattaatgtttaattacctggagcacctgcctgaaatcactttttttcaggttgg

SEQ ID NO: 9 - DNA, 469 bp, Homo sapiens
ctgcagagggccctgcgtatgagtgcaagtgggttttaggaccaggatgaggcggggtgggggtgcctacctgacgaccgaccccgacccactggacaagcacccaacccccattccccaaattgcgcatcccctatcagagagggggaggggaaacaggatgcggcgaggcgcgtgcgcactgccagcttcagcaccgcggacagtgccttcgcccccgcctggcggcgcgcgccaccgccgcctcagcactgaaggcgcgctgacgtcactcgccggtcccccgcaaactccccttcccggccaccttggtcgcgtccgcgccgccgccggcccagccggaccgcaccacgcgaggcgcgagataggggggcacgggcgcgaccatctgcgctgcggcgccggcgactcagcgctgcctcagtctgcggtgggcagcggaggagtcgtgtcgtgcctgagagcgcag

SEQ ID NO: 10 - DNA, 659 bp, Artificial Sequence
  Chicken beta actin promoter with a cytomegalovirus enhancer (CB7)
gacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcg

SEQ ID NO: 11 - DNA, 663 bp, Artificial Sequence
  TRE promoter
acgcgtggagctagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgtcaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttgcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctcc

SEQ ID NO: 12 - DNA, 592 bp, Woodchuck hepatitis B virus
aatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctg

SEQ ID NO: 13 - DNA, 247 bp, Woodchuck hepatitis B virus
aatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttagttcttgccacggcggaactcatcgccgcctgccttgcccgctgctggacaggggctcggctgttgggcactgacaattccgtggtgt

SEQ ID NO: 14 - DNA, 224 bp, Simian virus 40
agcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggta

SEQ ID NO: 15 - DNA, 208 bp, Bos taurus
ctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagagaatagcaggcatgctgggga

SEQ ID NO: 16 - DNA, 113 bp, Artificial Sequence
  ITR sequence
ccactccctctctgcgcgctcgctcgctcactgaggccgggcgaccaaaggtcgcccgacgcccgggctttgcccgggcggcctcagtgagcgagcgagcgcgcagagaggga

SEQ ID NO: 17 - DNA, 5071 bp, Artificial Sequence
  Expression vector comprising a CBh promoter operably linked to an htTH coding sequence
agcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagctctcgagatctagaaagcttcccggggggatctgggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcctggaggggtggagtcgtgacctaggcaactttgtatagaaaagttgcgttacataacttacggtaaatggcccgcctggctgaccgcccaacgacccccgcccattgacgtcaatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattgtgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagctgagcaagaggtaagggtttaagggatggttggttggtggggtattaatgtttaattacctggagcacctgcctgaaatcactttttttcaggttggcaagtttgtacagccaccatgagccccgcggggcccaaggtcccctggttcccaagaaaagtgtcagagctggacaagtgtcatcacctggtcaccaagttcgaccctgacctggacttggaccacccgggcttctcggaccaggtgtaccgccagcgcaggaagctgattgctgagatcgccttccagtacaggcacggcgacccgattccccgtgtggagtacaccgccgaggagattgccacctggaaggaggtctacaccacgctgaagggcctctacgccacgcacgcctgcggggagcacctggaggcctttgctttgctggagcgcttcagcggctaccgggaagacaatatcccccagctggaggacgtctcccgcttcctgaaggagcgcacgggcttccagctgcggcctgtggccggcctgctgtccgcccgggacttcctggccagcctggccttccgcgtgttccagtgcacccagtatatccgccacgcgtcctcgcccatgcactcccctgagccggactgctgccacgagctgctggggcacgtgcccatgctggccgaccgcaccttcgcgcagttctcgcaggacattggcctggcgtccctgggggcctcggatgaggaaattgagaagctgtccacgctgtactggttcacggtggagttcgggctgtgtaagcagaacggggaggtgaaggcctatggtgccgggctgctgtcctcctacggggagctcctgcactgcctgtctgaggagcctgagattcgggccttcgaccctgaggctgcggccgtgcagccctaccaagaccagacgtaccagtcagtctacttcgtgtctgagagcttcagtgacgccaaggacaagctcaggagctatgcctcacgcatccagcgccccttctccgtgaagttcgacccgtacacgctggccatcgacgtgctggacagcccccaggccgtgcggcgctccctggagggtgtccaggatgagctggacacccttgcccatgcgctgagtgccattggctaacagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtaactagtccactccctctctgcgcgctcgctcgctcactgaggccgggcgaccaaaggtcgcccgacgcccgggctttgcccgggcggcctcagtgagcgagcgagcgcgcagagagggacagatccgggcccgcatgcgtcgacaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaag

SEQ ID NO: 18 - DNA, 4954 bp, Artificial Sequence
  Expression vector comprising a CBh promoter operably linked to a GCH-1 coding sequence
agcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagctctcgagatctagaaagcttcccggggggatctgggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcctggaggggtggagtcgtgacctaggcaactttgtatagaaaagttgcgttacataacttacggtaaatggcccgcctggctgaccgcccaacgacccccgcccattgacgtcaatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattgtgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagctgagcaagaggtaagggtttaagggatggttggttggtggggtattaatgtttaattacctggagcacctgcctgaaatcactttttttcaggttggcaagtttgtacagccaccatggagaagccgcggggtgtaaggtgcaccaatgggttccccgagcgggagctgccgcggcccggggccagccgacctgccgagaagtcccggccgcccgaggccaagggcgcacagccagccgacgcctggaaggcagggcggccccgcagcgaggaggataacgagctgaacctccccaacctggcggccgcttactcgtccatcctgcgctcgctgggcgaggacccccagcggcaggggctgctcaagacgccctggagggcggccaccgccatgcagttcttcaccaagggataccaggagaccatctcagatgtcctgaacgatgctatatttgatgaggaccatgacgagatggtgattgtgaaggacattgacatgttttccatgtgtgagcatcacctggtcccatttgtgggaagggtccatattggttatcttcctaacaagcaagtccttggtctcagcaaacttgccaggattgtggaaatctacagtagaagactacaagttcaagaacgccttaccaaacagattgcagtggccatcacagaagccttgcagcctgctggcgtcggggtagtgattgaagcaacacacatgtgtatggtcatgcgaggtgtgcagaaaatgaacagcaagactgtcactagcaccatgctaggcgtgttccgggaagacccaaagactcgggaggagttcctcacactcatcaggagctgaaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttagttcttgccacggcggaactcatcgccgcctgccttgcccgctgctggacaggggctcggctgttgggcactgacaattccgtggtgtttatttgtgaaatttgtgatgctattgctttatttgtaaccatctagctttatttgtgaaatttgtgatgctattgctttatttgtaaccattataagctgcaataaacaagttaacaacaacaattgcattcattttatgtttcaggttcagggggagagactagtccactccctctctgcgcgctcgctcgctcactgaggccgggcgaccaaaggtcgcccgacgcccgggctttgcccgggcggcctcagtgagcgagcgagcgcgcagagagggacagatccgggcccgcatgcgtcgacaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaag

SEQ ID NO: 19 - DNA, 4984 bp, Artificial Sequence
  Expression vector comprising a SYN1 promoter operably linked to an htTH coding sequence
ggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcctggaggggtggagtcgtgacctaggcaactttgtatagaaaagttgctgcagagtgcaagtgggttttaggaccaggatgaggcggggtgggggtgcctacctgacgaccgaccccgacccactggacaagcacccaacccccattccccaaattgcgcatcccctatcagagagggggaggggaaacaggatgcggcgaggcgcgtgcgcactgccagcttcagcaccgcggacagtgccttcgcccccgcctggcggcgcgcgccaccgccgcctcagcactgaaggcgcgctgacgtcactcgccggtcccccgcaaactccccttcccggccaccttggtcgcgtccgcgccgccgccggcccagccggaccgcaccacgcgaggcgcgagataggggggcacgggcgcgaccatctgcgctgcggcgccggcgactcagcgctgcctcagtctgcggtgggcagcggaggagtcgtgtcgtgcctgagagcgcaggccaccatgagccccgcggggcccaaggtcccctggttcccaagaaaagtgtcagagctggacaagtgtcatcacctggtcaccaagttcgaccctgacctggacttggaccacccgggcttctcggaccaggtgtaccgccagcgcaggaagctgattgctgagatcgccttccagtacaggcacggcgacccgattccccgtgtggagtacaccgccgaggagattgccacctggaaggaggtctacaccacgctgaagggcctctacgccacgcacgcctgcggggagcacctggaggcctttgctttgctggagcgcttcagcggctaccgggaagacaatatcccccagctggaggacgtctcccgcttcctgaaggagcgcacgggcttccagctgcggcctgtggccggcctgctgtccgcccgggacttcctggccagcctggccttccgcgtgttccagtgcacccagtatatccgccacgcgtcctcgcccatgcactcccctgagccggactgctgccacgagctgctggggcacgtgcccatgctggccgaccgcaccttcgcgcagttctcgcaggacattggcctggcgtccctgggggcctcggatgaggaaattgagaagctgtccacgctgtactggttcacggtggagttcgggctgtgtaagcagaacggggaggtgaaggcctatggtgccgggctgctgtcctcctacggggagctcctgcactgcctgtctgaggagcctgagattcgggccttcgaccctgaggctgcggccgtgcagccctaccaagaccagacgtaccagtcagtctacttcgtgtctgagagcttcagtgacgccaaggacaagctcaggagctatgcctcacgcatccagcgccccttctccgtgaagttcgacccgtacacgctggccatcgacgtgctggacagcccccaggccgtgcggcgctccctggagggtgtccaggatgagctggacacccttgcccatgcgctgagtgccattggctaaaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttagttcttgccacggcggaactcatcgccgcctgccttgcccgctgctggacaggggctcggctgttgggcactgacaattccgtggtgtttatttgtgaaatttgtgatgctattgctttatttgtaaccatctagctttatttgtgaaatttgtgatgctattgctttatttgtaaccattataagctgcaataaacaagttaacaacaacaattgcattcattttatgtttcaggttcagggggagagcaattgcattcattttatgtttcaggttcagggggaggtgtgggaggttttttaaagcaagtaaaacctctacaaatgtggtaactagtccactccctctctgcgcgctcgctcgctcactgaggccgggcgaccaaaggtcgcccgacgcccgggctttgcccgggcggcctcagtgagcgagcgagcgcgcagagagggacagatccgggcccgcatgcgtcgacaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagctctcgagatctagaaagcttcccggggggatctg

SEQ ID NO: 20 - DNA, 4681 bp, Artificial Sequence
  Expression vector comprising a SYN1 promoter operably linked to a GCH-1 coding sequence
ggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcctggaggggtggagtcgtgacctaggcaactttgtatagaaaagttgctgcagagtgcaagtgggttttaggaccaggatgaggcggggtgggggtgcctacctgacgaccgaccccgacccactggacaagcacccaacccccattccccaaattgcgcatcccctatcagagagggggaggggaaacaggatgcggcgaggcgcgtgcgcactgccagcttcagcaccgcggacagtgccttcgcccccgcctggcggcgcgcgccaccgccgcctcagcactgaaggcgcgctgacgtcactcgccggtcccccgcaaactccccttcccggccaccttggtcgcgtccgcgccgccgccggcccagccggaccgcaccacgcgaggcgcgagataggggggcacgggcgcgaccatctgcgctgcggcgccggcgactcagcgctgcctcagtctgcggtgggcagcggaggagtcgtgtcgtgcctgagagcgcaggccaccatggagaagccgcggggtgtaaggtgcaccaatgggttccccgagcgggagctgccgcggcccggggccagccgacctgccgagaagtcccggccgcccgaggccaagggcgcacagccagccgacgcctggaaggcagggcggccccgcagcgaggaggataacgagctgaacctccccaacctggcggccgcttactcgtccatcctgcgctcgctgggcgaggacccccagcggcaggggctgctcaagacgccctggagggcggccaccgccatgcagttcttcaccaagggataccaggagaccatctcagatgtcctgaacgatgctatatttgatgaggaccatgacgagatggtgattgtgaaggacattgacatgttttccatgtgtgagcatcacctggtcccatttgtgggaagggtccatattggttatcttcctaacaagcaagtccttggtctcagcaaacttgccaggattgtggaaatctacagtagaagactacaagttcaagaacgccttaccaaacagattgcagtggccatcacagaagccttgcagcctgctggcgtcggggtagtgattgaagcaacacacatgtgtatggtcatgcgaggtgtgcagaaaatgaacagcaagactgtcactagcaccatgctaggcgtgttccgggaagacccaaagactcgggaggagttcctcacactcatcaggagctgaaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttagttcttgccacggcggaactcatcgccgcctgccttgcccgctgctggacaggggctcggctgttgggcactgacaattccgtggtgtttatttgtgaaatttgtgatgctattgctttatttgtaaccatctagctttatttgtgaaatttgtgatgctattgctttatttgtaaccattataagctgcaataaacaagttaacaacaacaattgcattcattttatgtttcaggttcagggggagagcaattgcattcattttatgtttcaggttcagggggaggtgtgggaggttttttaaagcaagtaaaacctctacaaatgtggtaactagtccactccctctctgcgcgctcgctcgctcactgaggccgggcgaccaaaggtcgcccgacgcccgggctttgcccgggcggcctcagtgagcgagcgagcgcgcagagagggacagatccgggcccgcatgcgtcgacaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagctctcgagatctagaaagcttcccggggggatctg

SEQ ID NO: 21 - DNA, 1494 bp, Homo sapiens
atgcccacccccgacgccaccacgccacaggccaagggcttccgcagggccgtgtctgagctggacgccaagcaggcagaggccatcatgtccccgcggttcattgggcgcaggcagagcctcatcgaggacgcccgcaaggagcgggaggcggcggtggcagcagcggccgctgcagtcccctcggagcccggggaccccctggaggctgtggcctttgaggagaaggaggggaaggccgtgctaaacctgctcttctccccgagggccaccaagccctcggcgctgtcccgagctgtgaaggtgtttgagacgtttgaagccaaaatccaccatctagagacccggcccgcccagaggccgcgagctgggggcccccacctggagtacttcgtgcgcctcgaggtgcgccgaggggacctggccgccctgctcagtggtgtgcgccaggtgtcagaggacgtgcgcagccccgcggggcccaaggtcccctggttcccaagaaaagtgtcagagctggacaagtgtcatcacctggtcaccaagttcgaccctgacctggacttggaccacccgggcttctcggaccaggtgtaccgccagcgcaggaagctgattgctgagatcgccttccagtacaggcacggcgacccgattccccgtgtggagtacaccgccgaggagattgccacctggaaggaggtctacaccacgctgaagggcctctacgccacgcacgcctgcggggagcacctggaggcctttgctttgctggagcgcttcagcggctaccgggaagacaatatcccccagctggaggacgtctcccgcttcctgaaggagcgcacgggcttccagctgcggcctgtggccggcctgctgtccgcccgggacttcctggccagcctggccttccgcgtgttccagtgcacccagtatatccgccacgcgtcctcgcccatgcactcccctgagccggactgctgccacgagctgctggggcacgtgcccatgctggccgaccgcaccttcgcgcagttctcgcaggacattggcctggcgtccctgggggcctcggatgaggaaattgagaagctgtccacgctgtcatggttcacggtggagttcgggctgtgtaagcagaacggggaggtgaaggcctatggtgccgggctgctgtcctcctacggggagctcctgcactgcctgtctgaggagcctgagattcgggccttcgaccctgaggctgcggccgtgcagccctaccaagaccagacgtaccagtcagtctacttcgtgtctgagagcttcagtgacgccaaggacaagctcaggagctatgcctcacgcatccagcgccccttctccgtgaagttcgacccgtacacgctggccatcgacgtgctggacagcccccaggccgtgcggcgctccctggagggtgtccaggatgagctggacacccttgcccatgcgctgagtgccattggctag

SEQ ID NO: 22 - protein, 497 aa, Homo sapiens
MPTPDATTPQAKGFRRAVSELDAKQAEAIMSPRFIGRRQSLIEDARKEREAAVAAAAAAVPSEPGDPLEAVAFEEKEGKAVLNLLFSPRATKPSALSRAVKVFETFEAKIHHLETRPAQRPRAGGPHLEYFVRLEVRRGDLAALLSGVRQVSEDVRSPAGPKVPWFPRKVSELDKCHHLVTKFDPDLDLDHPGFSDQVYRQRRKLIAEIAFQYRHGDPIPRVEYTAEEIATWKEVYTTLKGLYATHACGEHLEAFALLERFSGYREDNIPQLEDVSRFLKERTGFQLRPVAGLLSARDFLASLAFRVFQCTQYIRHASSPMHSPEPDCCHELLGHVPMLADRTFAQFSQDIGLASLGASDEEIEKLSTLSWFTVEFGLCKQNGEVKAYGAGLLSSYGELLHCLSEEPEIRAFDPEAAAVQPYQDQTYQSVYFVSESFSDAKDKLRSYASRIQRPFSVKFDPYTLAIDVLDSPQAVRRSLEGVQDELDTLAHALSAIG

SEQ ID NO: 23 - DNA, 1029 bp, Homo sapiens; identical to SEQ ID NO: 3

SEQ ID NO: 24 - protein, 342 aa, Homo sapiens
MSPAGPKVPWFPRKVSELDKCHHLVTKFDPDLDLDHPGFSDQVYRQRRKLIAEIAFQYRHGDPIPRVEYTAEEIATWKEVYTTLKGLYATHACGEHLEAFALLERFSGYREDNIPQLEDVSRFLKERTGFQLRPVAGLLSARDFLASLAFRVFQCTQYIRHASSPMHSPEPDCCHELLGHVPMLADRTFAQFSQDIGLASLGASDEEIEKLSTLSWFTVEFGLCKQNGEVKAYGAGLLSSYGELLHCLSEEPEIRAFDPEAAAVQPYQDQTYQSVYFVSESFSDAKDKLRSYASRIQRPFSVKFDPYTLAIDVLDSPQAVRRSLEGVQDELDTLAHALSAIG

SEQ ID NO: 25 - DNA, 589 bp, Artificial Sequence
  CMV promoter
tagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatc

SEQ ID NO: 26 - DNA, 272 bp, Homo sapiens
ttcgaacaggtaagcgcccctaaaatccctttgggcacaatgtgtcctgaggggagaggcagcgacctgtagatgggacgggggcactaaccctcaggtttggggcttctgaatgtgagtatcgccatgtaagcccagtatttggccaatctcagaaagctcctggtccctggagggatggagagagaaaaacaaacagctcctggagcagggagagtgctggcctcttgctctccggctccctctgttgccctctggtttctccccaggtt

SEQ ID NO: 27 - DNA, 82 bp, Mice minute virus
gtaagggtttaagggatggttggttggtggggtattaatgtttaattacctggagcacctgcctgaaatcactttttttcag

SEQ ID NO: 28 - DNA, 304 bp, Artificial Sequence
  3′UTR coding sequence
gtgcacggcgtccctgagggcccttcccaacctcccctggtcctgcactgtcccggagctcaggccctggtgaggggctgggtcccgggtgccccccatgccctccctgctgccaggctcccactgcccctgcacctgcttctcagcgcaacagctgtgtgtgcccgtggtgaggttgtgctgcctgtggtgaggtcctgtcctggctcccagggtcctgggggctgctgcactgccctccgcccttccctgacactgtctgctgccccaatcaccgtcacaataaaagaaactgtggtctcta

SEQ ID NO: 29 - DNA, 6 bp, Artificial Sequence
  5′UTR coding sequence
gccacc

(19) United States
(12) Patent Application Publication
(10) Pub. No.: US 2025/0277203 A1
(43) Pub. Date: Sep. 4, 2025

(54) EXPRESSION VECTORS COMPOSITION

(71) Applicant: MAAVRX LTD, Guildford (GB)

(72) Inventor: Michael McDONALD, Guildford Surrey (GB)

(73) Assignee: MAAVRX LTD, Guildford (GB)

(21) Appl. No.: 18/265,659

(22) PCT Filed: Dec. 7, 2021
(86) PCT No.: PCT/GB2021/053191

§ 371 Date: Jun. 6, 2023

(30) Foreign Application Priority Data
Dec. 8, 2020 (GB) 2019286.0

(51) Int. Cl. C12N 9/78; A61K 38/00; A61K 48/00; A61P 25/16; C12N 9/02; C12N 15/86

(52) U.S. Cl. CPC C12N 9/78; A61K 48/0058; A61K 48/0083; A61P 25/16; C12N 9/0071; C12N 15/86; A61K 38/00; C12N 2750/14143; C12N 2830/003; C12N 2830/008; C12N 2830/48; C12N 2830/50; C12Y 114/16002; C12Y 305/04016

(57) ABSTRACT

The invention relates to expression vectors, and pharmaceutical compositions, and kits comprising the vectors, and, in particular, their use in methods for treating Parkinson's disease (PD), DOPA responsive dystonia, vascular parkinsonism, side effects associated with L-DOPA treatment for Parkinson's disease, L-DOPA induced dyskinesia, Segawa syndrome, or genetic dopamine receptor abnormalities.

The present invention relates to expression vectors, and pharmaceutical compositions, and kits comprising the vectors, and, in particular, their use in methods for treating Parkinson's disease (PD), DOPA responsive dystonia, vascular parkinsonism, side effects associated with L-DOPA treatment for Parkinson's disease, L-DOPA induced dyskinesia, Segawa syndrome, or genetic dopamine receptor abnormalities.

Parkinson's disease is a neurodegenerative disease associated with the loss of dopamine-producing cells in the striatum. Three enzymes are necessary for the production of dopamine by brain cells: tyrosine hydroxylase (TH), GTP cyclohydrolase 1 (GCH1) and aromatic amino acid decarboxylase (AADC). TH and GCH1 regulate the production of L-DOPA (a precursor to dopamine) from tyrosine, and AADC converts L-DOPA to dopamine. The current treatment options for Parkinson's disease include oral administration of L-DOPA, which, in contrast to dopamine, is absorbed across the blood-brain barrier. This treatment is efficacious because AADC is still present in the brains of patients with Parkinson's disease.

However, a problem with oral L-DOPA therapy is that it can lead to side effects, such as abnormal movement. These side effects are believed to be due to the fluctuation of levels of L-DOPA in the blood and brain caused by the short half-life of L-DOPA and the variable absorption across the gut mucosa and blood brain barrier resulting from competition with other amino acids for active transport (Lees, April 2008, The Importance of Steady-State plasma DOPA levels in reducing motor fluctuations in Parkinson's disease, Expert Roundtable Supplement, CNS Spectr 13:4 (Suppl 7) P4-7).

Many attempts have been made to formulate L-DOPA into a sustained release oral product that will deliver steady blood and brain levels of L-DOPA. These have not been successful. Currently, the most effective method for delivering steady plasma L-DOPA level requires constant slow infusion of a gel formulation of L-DOPA directly into the patient's jejunum via a tube through the patient's abdominal wall. The more stable plasma levels of L-DOPA result in significantly improved symptomatic control and reduced dyskinesias (Olanow et al Continuous intrajejunal infusion of levodopa-carbidopa intestinal gel for patients with advanced Parkinson's disease: a randomised, controlled, double-blind, double-dummy study. The Lancet Neurology Vol 13 Feb. 2014). However the lifelong requirement for a tube through the abdominal wall (with adverse events including dislodgement, kinking, blockage and infection), to carry a large pump and to refresh the supply of gel daily restrict use of this therapy and make it suboptimal especially for elderly patients with PD.

Many attempts, therefore, have, been made by multiple authors to restore dopamine levels in Parkinson's disease patients by targeting gene therapy directly into the most affected area of the brain, i.e. the striatum. Preclinical and clinical studies have shown some effect with various constructs but none has yet demonstrated sufficient efficacy, safety or the ability to be manufactured at a commercially viable cost to be approved for medicinal use in any country.

Efficacy of a formulation of a mixture of three monocistronic single stranded AAV vectors each encoding either TH, GCH, or AADC demonstrated efficacy in the 1-methyl-4-phenyl-1,2,3,6-tetrahydropyridine (MPTP) lesioned macaque model of PD. The contribution of the AAV vector encoding AADC was considered integral to the success of the formulation. This product was never progressed to clinical evaluation (Muramatsu 10 February, 2002, Behavioural Recovery in a Primate Model of Parkinson's disease by Triple Transduction of Striatal Cells with Adeno-Associated Viral (AAV) Vectors Expressing dopamine-Synthesizing Enzymes, Human Gene Therapy, 12: 345-354). The requirement to produce and mix three different AAV vectors and the resulting cost of goods was a factor in the decision not to develop the formulation further.

A single tricistronic Lente vector encoding all three genes demonstrated efficacy in the MPTP macaque model of PD (Azzouz M, Martin-Rendon E, Barber R D, et al. Multicistronic Lentiviral Vector-Mediated Striatal Gene Transfer of Aromatic 1-Amino Acid Decarboxylase, Tyrosine Hydroxylase, and GTP Cyclohydrolase I Induces Sustained Transgene Expression, Dopamine Production, and Functional Improvement in a Rat Model of Parkinson's Disease. J Neurosci. 2002; 22(23):10302-10312.). Although this was progressed into a clinical trial, the reported efficacy was modest. Approximately one third of treated patients reported no reduction in their oral L-DOPA equivalent daily dose of L-DOPA and dopamine agonists. Remaining patients experienced only a modest reduction in the need for oral L-DOPA or dopamine agonists. The most common reported adverse events remained dyskinesia and on/off fluctuation (Palfi S, Gurru J, Le H, et al. Long-term follow up of a phase 1/2 study of ProSavin, a lentiviral vector gene therapy for Parkinson's disease. Hum Gene Ther Cl Dev. Published online 2018.). Again, the contribution of the AADC was considered integral to the efficacy of the product. However, AADC may also have contributed to the incidence of dyskinesia by locally amplifying the erratic peaks in L-DOPA levels associated with the continued requirement for oral L-DOPA.

Rosenblad et al evaluated a bicistronic AAV encoding only TH and GCH1 administered directly to the striatum to produce L-DOPA (WO2013/061076 and WO2010/055209). Although this bicistronic AAV vector resulted in strong expression of TH and GCH1 and complete symptomatic improvement of motor deficit in the 6-hydroxydopamine (6-OHDA) lesioned rat model of PD, it did not result in the expected increase in striatal TH expression (assessed by immunohistochemistry) or sufficient motor improvement in the MPTP lesioned macaque model of PD leading the inventors to write “This issue should be resolved prior to proceeding towards clinical trials.” (Cederfjill E, Nilsson N, Sahin G, et al. Continuous DOPA synthesis from a single AAV: dosing and efficacy in models of Parkinson's disease. Sci Rep-uk. 2013; 3(1).). Furthermore, the inventors stated “we cannot exclude that this property of our vector is due to some feature of its design” (Rosenblad C, Li Q, Pioli E Y, et al. Vector-mediated 1-3,4-dihydroxyphenylalanine delivery reverses motor impairments in a primate model of Parkinson's disease. Brain. 2019; 142(8):2402-2416.).

Despite this prior art, there clearly remains an unmet clinical need for an effective gene therapy to treat PD.

The inventors investigated novel expression vectors for treating PD, and found that using a combination of two monocistronic vectors, which separately encode GCH1 and TH1, results in surprising levels of gene expression. This approach is superior to that of Rosenblad as, in contrast to the Rosenblad bicistronic vector, it resulted in TH expression which was readily detected by immunohistochemistry. In contrast to the Muramatsu and Azzouz methods the invention results in improved motor function in the MPTP lesioned macaque model of PD without augmenting AADC.

Thus, according to a first aspect of the invention, there is provided a composition comprising first and second expression vectors, wherein the first expression vector comprises a self-complementary coding sequence including a promoter operably linked to a sequence, which encodes tyrosine hydroxylase (TH), and the second expression vector comprises a self-complementary coding sequence including a promoter operably linked to a coding sequence, which encodes GTP cyclohydrolase 1 (GCH1).

Preferably, the composition does not comprise a vector (either the first or second expression vector or any other vector), which encodes aromatic amino acid decarboxylase (AADC).

Advantageously, the composition of the invention exhibits the following unique combination of properties:
a) It does not require the manufacture and mixing of three vectors (it only requires two vectors), thus simplifying manufacture and reducing cost;
b) It does not encode AADC, thus reducing the potential for increased AADC expression to exaggerate peak dopamine levels in a patient with a continued requirement for oral L-DOPA (albeit at reduced dosage); and
c) It results in strong expression of TH, for example in the striatum, thereby resulting in restored levels of endogenously produced L-DOPA and dopamine for use in treating Parkinson's disease.

Preferably, in one embodiment, the first expression vector is an AAV vector. Preferably, in one embodiment, the second expression vector is an AAV vector.

Preferably, in one embodiment, the first expression vector is a self-complementary AAV (scAAV) vector. Preferably, in one embodiment, the second expression vector is a self-complementary AAV vector.

In another embodiment, the first expression vector is a naked DNA vector. Preferably, the second expression vector is a naked DNA vector.

Preferably, in one embodiment, the second expression vector is a single-stranded AAV (ssAAV) vector.

In one preferred embodiment, however, the first expression vector is self-complementary AAV vector, and the second expression vector is a ssAAV or naked DNA vector.

The skilled person would understand that a self-complementary vector is a vector that includes an inverted repeat genome that can fold into dsDNA without the requirement for DNA synthesis or base-pairing between multiple vector genomes. A self-complementary adeno-associated virus (scAAV) vector is an adeno-associated virus (AAV) vector that carries an inverted repeat genome that can fold into dsDNA without the requirement for DNA synthesis or base-pairing between multiple vector genomes

The AAV (first and/or second expression vector) may be derived from AAV-1, AAV-2, AAV-3A, AAV-3B, AAV-4, AAV-5, AAV-6, AAV-7, AAV-8, AAV-9, AAV-10, and/or AAV-11. Preferably, the AAV has tropism to neural tissue. The first and second AAV expression vectors may be different serotypes, but more preferably are the same serotype. In a preferred embodiment, the AAV (first and/or second expression vector) may be derived from AAV1, AAV5, or AAV9, and more preferably, AAV5.

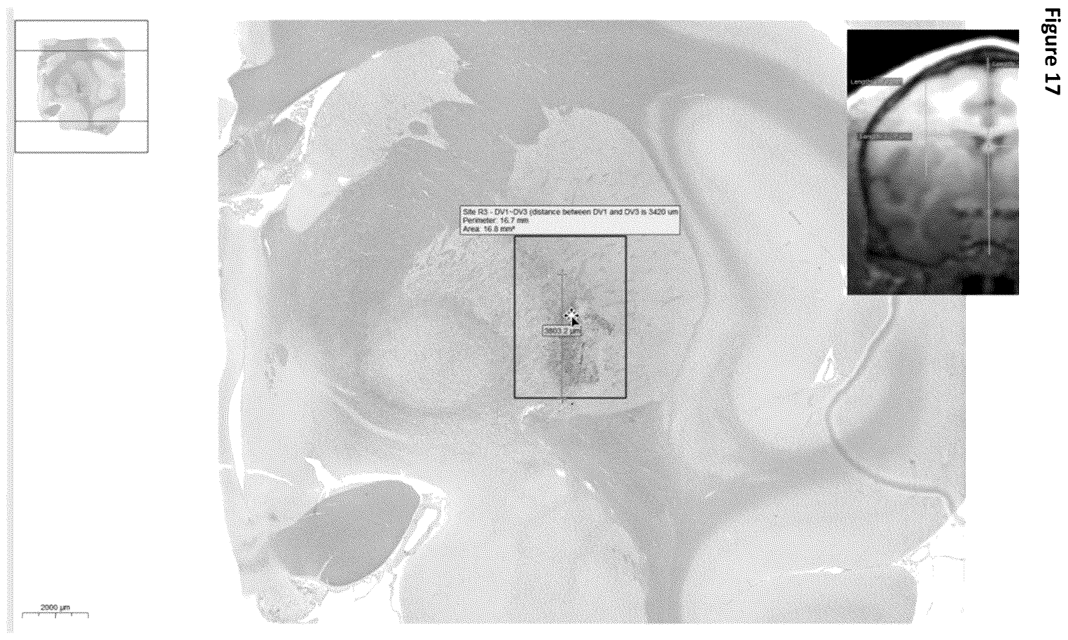

As described in the Examples, and as shown in FIGS. 17-19, using a 1:1 mixture of scAAV5-human truncated TH and scAAV5-GCH1 (both using CBh promoters) resulted in very clear expression of human truncated TH. Thus, AAV5 is most preferred for the first and/or second expression vector. The skilled person would understand that the truncated TH lacks the regulatory domain of TH.

The composition is preferably a combination or mixture of the first and second expression vectors. The TH-encoding vector is preferably self-complementary AAV (scAAV). The GCH-encoding vector may be either self-complementary or single stranded. Preferably, however, the second vector is also self-complementary. Therefore, in a most preferred embodiment, the first expression vector is scAAV and the second expression vector is scAAV.

It should be appreciated that based on prior art it was not at all predictable or expected that administration of two different vectors would result is sufficient coinfection of sufficient cells at an effective ratio throughout a sufficient volume of the primate striatum to achieve sufficient new transduced L-DOPA production to achieve impromved motor function in the MPTP macaque model of PD. Furthermore, the use of scAAVs results in significantly lower doses being required due to their high expression levels, which reduces the cost of the treatment, which is very important given the high price of AAVs.

The composition may comprise the two vectors supplied individually (e.g. in a vial or syringe) and mixed immediately prior to, or at the time of, administration, or may be supplied as a single pre-mixed formulation. The ratio of the first vector to second vector may preferably be about 50:50, but could be 5:95, 10:90, 20:80, 30:70 60:40, 40:60, 70:30, 80:20, 90:10 or 95:5.

In one embodiment, the coding sequence, which encodes TH, encodes human TH, which is referred to herein as SEQ ID No: 1, as set out below:

[SEQ ID NO: 1]
atgcccacccccgacgccaccacgccacaggccaagggcttccgcagggccgtgtctgagctggacgccaagcaggca
gaggccatcatgtccccgcggttcattgggcgcaggcagagcctcatcgaggacgcccgcaaggagcgggaggcggcg
gtggcagcagcggccgctgcagtcccctcggagcccggggaccccctggaggctgtggcctttgaggagaaggagggg
aaggccgtgctaaacctgctcttctccccgagggccaccaagccctcggcgctgtcccgagctgtgaaggtgtttgag
acgtttgaagccaaaatccaccatctagagacccggcccgcccagaggccgcgagctgggggcccccacctggagtac
ttcgtgcgcctcgaggtgcgccgaggggacctggccgccctgctcagtggtgtgcgccaggtgtcagaggacgtgcgc
agccccgcggggcccaaggtcccctggttcccaagaaaagtgtcagagctggacaagtgtcatcacctggtcaccaag
ttcgaccctgacctggacttggaccacccgggcttctcggaccaggtgtaccgccagcgcaggaagctgattgctgag
atcgccttccagtacaggcacggcgacccgattccccgtgtggagtacaccgccgaggagattgccacctggaaggag
gtctacaccacgctgaagggcctctacgccacgcacgcctgcggggagcacctggaggcctttgctttgctggagcgc
ttcagcggctaccgggaagacaatatcccccagctggaggacgtctcccgcttcctgaaggagcgcacgggcttccag
ctgcggcctgtggccggcctgctgtccgcccgggacttcctggccagcctggccttccgcgtgttccagtgcacccag
tatatccgccacgcgtcctcgcccatgcactcccctgagccggactgctgccacgagctgctggggcacgtgcccatg
ctggccgaccgcaccttcgcgcagttctcgcaggacattggcctggcgtccctgggggcctcggatgaggaaattgag
aagctgtccacgctgtactggttcacggtggagttcgggctgtgtaagcagaacggggaggtgaaggcctatggtgcc
gggctgctgtcctcctacggggagctcctgcactgcctgtctgaggagcctgagattcgggccttcgaccctgaggct
gcggccgtgcagccctaccaagaccagacgtaccagtcagtctacttcgtgtctgagagcttcagtgacgccaaggac
aagctcaggagctatgcctcacgcatccagcgccccttctccgtgaagttcgacccgtacacgctggccatcgacgtg
ctggacagcccccaggccgtgcggcgctccctggagggtgtccaggatgagctggacacccttgcccatgcgctgagt
gccattggctag

Preferably, therefore, the coding sequence that encodes TH comprises a nucleotide sequence substantially as set out in SEQ ID No: 1, or a fragment or variant thereof.

Human TH may have an amino acid sequence according to NCBI Reference Sequence: NP_000351.2, which is referred to herein as SEQ ID NO: 2, as set out below:

[SEQ ID NO: 2]
MPTPDATTPQAKGFRRAVSELDAKQAEAIMSPRFIGRRQSLIEDARKEREAAVAAAAAAVPSEPGDPLEA
VAFEEKEGKAVLNLLESPRATKPSALSRAVKVFETFEAKIHHLETRPAQRPRAGGPHLEYFVRLEVRRGD
LAALLSGVRQVSEDVRSPAGPKVPWFPRKVSELDKCHHLVTKFDPDLDLDHPGFSDQVYRQRRKLIAEIA
FQYRHGDPIPRVEYTAEEIATWKEVYTTLKGLYATHACGEHLEAFALLERFSGYREDNIPQLEDVSRFLK
ERTGFQLRPVAGLLSARDFLASLAFRVFQCTQYIRHASSPMHSPEPDCCHELLGHVPMLADRTFAQFSQD
IGLASLGASDEEIEKLSTLYWFTVEFGLCKQNGEVKAYGAGLLSSYGELLHCLSEEPEIRAFDPEAAAVQ
PYQDQTYQSVYFVSESFSDAKDKLRSYASRIQRPFSVKFDPYTLAIDVLDSPQAVRRSLEGVQDELDTLA
HALSAIG*

Preferably, therefore, the coding sequence that encodes TH comprises a nucleotide sequence which encodes an amino acid sequence substantially as set out in SEQ ID No: 2, or a fragment or variant thereof.

In another embodiment, however, the coding sequence, which encodes TH, encodes human TH, which is referred to herein as SEQ ID No: 21, as set out below:

[SEQ ID NO: 21]
atgcccacccccgacgccaccacgccacaggccaagggcttccgcagggccgtgtctgagctggacgccaagcaggca
gaggccatcatgtccccgcggttcattgggcgcaggcagagcctcatcgaggacgcccgcaaggagcgggaggcggcg
gtggcagcagcggccgctgcagtcccctcggagcccggggaccccctggaggctgtggcctttgaggagaaggagggg
aaggccgtgctaaacctgctcttctccccgagggccaccaagccctcggcgctgtcccgagctgtgaaggtgtttgag
acgtttgaagccaaaatccaccatctagagacccggcccgcccagaggccgcgagctgggggcccccacctggagtac
ttcgtgcgcctcgaggtgcgccgaggggacctggccgccctgctcagtggtgtgcgccaggtgtcagaggacgtgcgc
agccccgcggggcccaaggtcccctggttcccaagaaaagtgtcagagctggacaagtgtcatcacctggtcaccaag
ttcgaccctgacctggacttggaccacccgggcttctcggaccaggtgtaccgccagcgcaggaagctgattgctgag
atcgccttccagtacaggcacggcgacccgattccccgtgtggagtacaccgccgaggagattgccacctggaaggag
gtctacaccacgctgaagggcctctacgccacgcacgcctgcggggagcacctggaggcctttgctttgctggagcgc
ttcagcggctaccgggaagacaatatcccccagctggaggacgtctcccgcttcctgaaggagcgcacgggcttccag
ctgcggcctgtggccggcctgctgtccgcccgggacttcctggccagcctggccttccgcgtgttccagtgcacccag
tatatccgccacgcgtcctcgcccatgcactcccctgagccggactgctgccacgagctgctggggcacgtgcccatg
ctggccgaccgcaccttcgcgcagttctcgcaggacattggcctggcgtccctgggggcctcggatgaggaaattgag
aagctgtccacgctgtcatggttcacggtggagttcgggctgtgtaagcagaacggggaggtgaaggcctatggtgcc
gggctgctgtcctcctacggggagctcctgcactgcctgtctgaggagcctgagattcgggccttcgaccctgaggct
gcggccgtgcagccctaccaagaccagacgtaccagtcagtctacttcgtgtctgagagcttcagtgacgccaaggac
aagctcaggagctatgcctcacgcatccagcgccccttctccgtgaagttcgacccgtacacgctggccatcgacgtg
ctggacagcccccaggccgtgcggcgctccctggagggtgtccaggatgagctggacacccttgcccatgcgctgagt
gccattggctag

It should be appreciated that SEQ ID No: 21 is exactly the same as SEQ ID No: 1, except for reversing AC (in SEQ ID No: 1) to CA (in SEQ ID No: 21) at positions 1109 and 1110. Preferably, therefore, the coding sequence that encodes TH comprises a nucleotide sequence substantially as set out in SEQ ID No: 21, or a fragment or variant thereof. Human TH encoded by SEQ ID No: 21 may have an amino acid sequence, which is referred to herein as SEQ ID NO: 22, as set out below:

[SEQ ID NO: 22]
MPTPDATTPQAKGFRRAVSELDAKQAEAIMSPRFIGRRQSLIEDARKEREAAVAAAAAAVPSEPGDPLEAVAFEEKEG
KAVLNLLFSPRATKPSALSRAVKVFETFEAKIHHLETRPAQRPRAGGPHLEYFVRLEVRRGDLAALLSGVRQVSEDVR
SPAGPKVPWFPRKVSELDKCHHLVTKFDPDLDLDHPGFSDQVYRQRRKLIAEIAFQYRHGDPIPRVEYTAEEIATWKE
VYTTLKGLYATHACGEHLEAFALLERFSGYREDNIPQLEDVSRFLKERTGFQLRPVAGLLSARDFLASLAFRVFQCTQ
YIRHASSPMHSPEPDCCHELLGHVPMLADRTFAQFSQDIGLASLGASDEEIEKLSTLSWFTVEFGLCKQNGEVKAYGA
GLLSSYGELLHCLSEEPEIRAFDPEAAAVQPYQDQTYQSVYFVSESFSDAKDKLRSYASRIQRPFSVKFDPYTLAIDV
LDSPQAVRRSLEGVQDELDTLAHALSAIG*

The A-C translocation at positions 1109 and 1110 (from SEQ ID No: 1 to SEQ ID No: 21) results in a change of amino acid 401 from a tyrosine to a serine. Preferably, therefore, the coding sequence that encodes TH comprises a nucleotide sequence which encodes an amino acid sequence substantially as set out in SEQ ID No: 22, or a fragment or variant thereof.

In another embodiment, however, the coding sequence encoding TH comprises a nucleotide sequence encoding human truncated TH. Human truncated TH is a variant of TH with the regulatory domain removed. Hence, preferably the first vector comprises a coding sequence encoding TH lacking the regulatory domain of TH. Advantageously, the first expression vector in the composition of the invention does not encode the regulatory domain of TH, and thus limits the potential for the resulting L-DOPA or dopamine to inhibit additional production due to feedback inhibition. It will be appreciated that the preferred embodiment of the construct comprises a nucleotide sequence encoding human truncated TH, because, in some embodiments, the non-truncated version may be too long to optimally fit into a scAAV.

The domains of TH and their roles are described in Daubner et al. (Daubner S C, Lohse D L, Fitzpatrick' P F. Expression and characterization of catalytic and regulatory domains of rat tyrosine hydroxylase. Protein Sci. 1993; 2:1452-60). In one embodiment, human truncated TH comprises the nucleotide sequence referred to herein as SEQ ID No: 3, as set out below:

[SEQ ID NO: 3]
atgagccccgcggggcccaaggtcccctggttcccaagaaaagtgtcagagctggacaagtgtcatcacctggtcacc
aagttcgaccctgacctggacttggaccacccgggcttctcggaccaggtgtaccgccagcgcaggaagctgattgct
gagatcgccttccagtacaggcacggcgacccgattccccgtgtggagtacaccgccgaggagattgccacctggaag
gaggtctacaccacgctgaagggcctctacgccacgcacgcctgcggggagcacctggaggcctttgctttgctggag
cgcttcagcggctaccgggaagacaatatcccccagctggaggacgtctcccgcttcctgaaggagcgcacgggcttc
cagctgcggcctgtggccggcctgctgtccgcccgggacttcctggccagcctggccttccgcgtgttccagtgcacc
cagtatatccgccacgcgtcctcgcccatgcactcccctgagccggactgctgccacgagctgctggggcacgtgccc
atgctggccgaccgcaccttcgcgcagttctcgcaggacattggcctggcgtccctgggggcctcggatgaggaaatt
gagaagctgtccacgctgtactggttcacggtggagttcgggctgtgtaagcagaacggggaggtgaaggcctatggt
gccgggctgctgtcctcctacggggagctcctgcactgcctgtctgaggagcctgagattcgggccttcgaccctgag
gctgcggccgtgcagccctaccaagaccagacgtaccagtcagtctacttcgtgtctgagagcttcagtgacgccaag
gacaagctcaggagctatgcctcacgcatccagcgccccttctccgtgaagttcgacccgtacacgctggccatcgac
gtgctggacagcccccaggccgtgcggcgctccctggagggtgtccaggatgagctggacacccttgcccatgcgctg
agtgccattggctag

Preferably, therefore, the coding sequence encoding TH (and preferably lacking the regulatory domain of TH) comprises a nucleotide sequence substantially as set out in SEQ ID No: 3, or a fragment or variant thereof.

In one preferred embodiment, the coding sequence encoding TH comprises a nucleotide sequence encoding human truncated TH. In one embodiment, human truncated TH comprises an amino acid sequence referred to herein as SEQ ID NO: 4, as set out below:

[SEQ ID NO: 4]
MSPAGPKVPWFPRKVSELDKCHHLVTKFDPDLDLDHPGFSDQVYRQRRKLIAEIAFQYRHGDPIPRVEYTAEEIATWK
EVYTTLKGLYATHACGEHLEAFALLERFSGYREDNIPQLEDVSRFLKERTGFQLRPVAGLLSARDFLASLAFRVFQCT
QYIRHASSPMHSPEPDCCHELLGHVPMLADRTFAQFSQDIGLASLGASDEEIEKLSTLYWFTVEFGLCKQNGEVKAYG
AGLLSSYGELLHCLSEEPEIRAFDPEAAAVQPYQDQTYQSVYFVSESFSDAKDKLRSYASRIQRPFSVKFDPYTLAID
VLDSPQAVRRSLEGVQDELDTLAHALSAIG*

Preferably, therefore, the coding sequence encoding TH comprises a nucleotide sequence encoding an amino acid sequence substantially as set out in SEQ ID No: 4, or a fragment or variant thereof.

In another embodiment, human truncated TH (having the AC to CA translocation) comprises the nucleotide sequence referred to herein as SEQ ID No: 23, as set out below:

[SEQ ID NO: 23]
Atgagccccgcggggcccaaggtcccctggttcccaagaaaagtgtcagagctggacaagtgtcatcacctggtcacc
aagttcgaccctgacctggacttggaccacccgggcttctcggaccaggtgtaccgccagcgcaggaagctgattgct
gagatcgccttccagtacaggcacggcgacccgattccccgtgtggagtacaccgccgaggagattgccacctggaag
gaggtctacaccacgctgaagggcctctacgccacgcacgcctgcggggagcacctggaggcctttgctttgctggag
cgcttcagcggctaccgggaagacaatatcccccagctggaggacgtctcccgcttcctgaaggagcgcacgggcttc
cagctgcggcctgtggccggcctgctgtccgcccgggacttcctggccagcctggccttccgcgtgttccagtgcacc
cagtatatccgccacgcgtcctcgcccatgcactcccctgagccggactgctgccacgagctgctggggcacgtgccc
atgctggccgaccgcaccttcgcgcagttctcgcaggacattggcctggcgtccctgggggcctcggatgaggaaatt
gagaagctgtccacgctgtactggttcacggtggagttcgggctgtgtaagcagaacggggaggtgaaggcctatggt
gccgggctgctgtcctcctacggggagctcctgcactgcctgtctgaggagcctgagattcgggccttcgaccctgag
gctgcggccgtgcagccctaccaagaccagacgtaccagtcagtctacttcgtgtctgagagcttcagtgacgccaag
gacaagctcaggagctatgcctcacgcatccagcgccccttctccgtgaagttcgacccgtacacgctggccatcgac
gtgctggacagcccccaggccgtgcggcgctccctggagggtgtccaggatgagctggacacccttgcccatgcgctg
agtgccattggctag

Preferably, the coding sequence encoding TH (and preferably lacking the regulatory domain of TH) comprises a nucleotide sequence substantially as set out in SEQ ID No: 23, or a fragment or variant thereof.

In another embodiment, human truncated TH (in which amino acid 401 has changed from a tyrosine to a serine) comprises an amino acid sequence referred to herein as SEQ ID NO: 24, as set out below:

[SEQ ID NO: 24]
MSPAGPKVPWFPRKVSELDKCHHLVTKFDPDLDLDHPGFSDQVYRQRRKLIAEIAFQYRHGDPIPRVEYTAEEIATWK
EVYTTLKGLYATHACGEHLEAFALLERFSGYREDNIPQLEDVSRFLKERTGFQLRPVAGLLSARDFLASLAFRVFQCT
QYIRHASSPMHSPEPDCCHELLGHVPMLADRTFAQFSQDIGLASLGASDEEIEKLSTLSWFTVEFGLCKQNGEVKAYG
AGLLSSYGELLHCLSEEPEIRAFDPEAAAVQPYQDQTYQSVYFVSESFSDAKDKLRSYASRIQRPFSVKEDPYTLAID
VLDSPQAVRRSLEGVQDELDTLAHALSAIG

Preferably, therefore, the coding sequence encoding TH comprises a nucleotide sequence encoding an amino acid sequence substantially as set out in SEQ ID No: 24, or a fragment or variant thereof.

In an embodiment, the coding sequence encoding GCH1 comprises a nucleotide sequence encoding murine GCH1. The nucleotide sequence encoding murine GCH1 is referred to herein as SEQ ID No: 5, as set out below:

[SEQ ID NO: 5]
ggtggttttcctttgaaaaacacgatgataatatggccacaaccgcggccgtagatcccgggaccatggagaagccgc
ggggagtcaggtgcaccaatgggttctccgagcgggagctgccgcggcccggggccagcccgcctgccgagaagtccc
ggccgcccgaggccaagggcgcacagccggccgacgcctggaaggcagggcggcaccgcagcgaggaggaaaaccagg
tgaacctccccaaactggcggctgcttactcgtccattctgctctcgctgggcgaggacccccagcggcaggggctgc
tcaagacgccctggagggcggccaccgccatgcagtacttcaccaagggataccaggagaccatctcagatgtcctga
atgatgctatatttgatgaagatcatgacgagatggtgattgtgaaggacatagatatgttctccatgtgtgagcatc
accttgttccatttgtaggaagggtccatattggctatcttcctaacaagcaagtccttggtctcagtaaacttgcca
ggattgtagaaatctacagtagacgactacaagttcaagagcgcctcaccaaacagattgcggtggccatcacagaag
ccttgcagcctgctggcgttggagtagtgattgaagcgacacacatgtgcatggtaatgcgaggcgtgcagaaaatga
acagcaagactgtcactagcaccatgctgggcgtgttccgggaagaccccaagactcgggaggagttcctcacactaa
tcaggagctgag

Therefore, the coding sequence may comprise a nucleotide sequence substantially as set out in SEQ ID No: 5, or a fragment or variant thereof.

In a preferred embodiment, however, the coding sequence encoding GCH1 comprises a nucleotide sequence encoding human GCH1. For example, the nucleotide sequence encoding human GCH may be the sequence according to GenBank NM 000161.2, which is referred to herein as SEQ ID No: 6, as set out below:

[SEQ ID NO: 6]
atggagaagggccctgtgcgggcaccggcggagaagccgcggggcgccaggtgcagcaatgggttccccg
agcgggatccgccgcggcccgggcccagcaggccggcggagaagcccccgcggcccgaggccaagagcgc
gcagcccgcggacggctggaagggcgagcggccccgcagcgaggaggataacgagctgaacctccctaac
ctggcagccgcctactcgtccatcctgagctcgctgggcgagaacccccagcggcaagggctgctcaaga
cgccctggagggcggcctcggccatgcagttcttcaccaagggctaccaggagaccatctcagatgtcct
aaacgatgctatatttgatgaagatcatgatgagatggtgattgtgaaggacatagacatgttttccatg
tgtgagcatcacttggttccatttgttggaaaggtccatattggttatcttcctaacaagcaagtccttg
gcctcagcaaacttgcgaggattgtagaaatctatagtagaagactacaagttcaggagcgccttacaaa
acaaattgctgtagcaatcacggaagccttgcggcctgctggagtcggggtagtggttgaagcaacacac
atgtgtatggtaatgcgaggtgtacagaaaatgaacagcaaaactgtgaccagcacaatgttgggtgtgt
tccgggaggatccaaagactcgggaagagttcctgactctcattaggagctga

Preferably, therefore, the coding sequence encoding GCH1 comprises a nucleotide sequence substantially as set out in SEQ ID No: 6, or a fragment or variant thereof.

In one preferred embodiment, the coding sequence encoding GCH1 comprises a nucleotide sequence encoding human GCH1. Human GCH1 may have an amino acid sequence according to NCBI Reference Sequence: NP_000152.1. Human GCH1 comprises an amino acid sequence referred to herein as SEQ ID NO: 7, as set out below:

[SEQ ID NO: 7]
MEKGPVRAPAEKPRGARCSNGFPERDPPRPGPSRPAEKPPRPEAKSAQPADGWKGERPRSEEDNELNLPN
LAAAYSSILSSLGENPQRQGLLKTPWRAASAMQFFTKGYQETISDVLNDAIFDEDHDEMVIVKDIDMFSM
CEHHLVPFVGKVHIGYLPNKQVLGLSKLARIVEIYSRRLQVQERLTKQIAVAITEALRPAGVGVVVEATH
MCMVMRGVQKMNSKTVTSTMLGVFREDPKTREEFLTLIRS*

Preferably, therefore, the coding sequence encoding GCH1 comprises a nucleotide sequence encoding an amino acid sequence substantially as set out in SEQ ID No: 7, or a fragment or variant thereof.

The first and second expression vectors each comprise a promoter which may be any suitable promoter, including a constitutive promoter, an activatable promoter, an inducible promoter, or a tissue-specific promoter. The promoter may be the same in the first construct and the second construct, or different promoters may be used for each construct.

In a preferred embodiment, the promoter in the first and second expression vector is one enabling the expression of TH and/or GCH1 in the most suitable tissue or tissues for treating Parkinson's disease. In an embodiment, therefore, the promoter is one that permits high expression in a subject's neurons, or in the subject's glial cells, or in the subject's neurons and glial cells, or in the subject's neurons and ependymal cells lining the cerebral ventricles, or in the subject's neurons and glial cells and ependymal cells.

Preferably, the promoter in the first and/or second expression vector may be the CBh promoter, or a fragment or variant thereof. (Gray S J, Foti S B, Schwartz J W, et al. Optimizing Promoters for Recombinant Adeno-Associated Virus-Mediated Gene Expression in the Peripheral and Central Nervous System Using Self-Complementary Vectors. Hum Gene Ther. 2011; 22(9):1143-1153.). As described in the Examples, the inventor has compared different potential constructs and demonstrated surprisingly that despite the limited packaging capacity of self-complementary vectors requiring the use of two different viral vectors (one to transduce TH and one to transduce GCH1), the increased transduction achieved requires a lower total number of vector genome copies than would be required using the optimal single bicistronic viral vector. This is important for at least two reasons: (1) reducing manufacturing cost of goods, and (2) reducing the AAV capsid load for the patients and thus reducing the risk of capsid related toxicity.

Either or both promoters in the expression vectors may be the CBh promoter. Preferably, both the first and second expression vector comprises the CBh promoter.

The use of the CBh promoter offers at least four advantages to the constructs for treating PD. Firstly, its short length enables accommodation of the promoter trans gene combination within a self-complementary AAV construct. Second, it is less prone to silencing than the CMV promoter which is widely used in previous monocistronic constructs. Thirdly, its lack of neuronal specificity enables transduction of astrocytes and glia increasing the potential for additional production of DOPA by these cells within the striatum. Fourthly, the CBh promoter contains both a truncated chicken beta-actin intron and a minute virus of mouse (MVM) intron, which, together, act as a spacer, thereby increasing gene expression.

In one embodiment, the sequence of the CBh promoter is referred to herein as SEQ ID NO: 8, as follows:

[SEQ ID No: 8]
CGTTACATAACTTACGGTAAATGGCCCGCCTGGCTGACCGCCCAACGACCCCCGCCCATTGACGTCAATAGTAACGCCAATAGGGACT
TTCCATTGACGTCAATGGGTGGAGTATTTACGGTAAACTGCCCACTTGGCAGTACATCAAGTGTATCATATGCCAAGTACGCCCCCTA
TTGACGTCAATGACGGTAAATGGCCCGCCTGGCATTGTGCCCAGTACATGACCTTATGGGACTTTCCTACTTGGCAGTACATCTACGT
ATTAGTCATCGCTATTACCATGGTCGAGGTGAGCCCCACGTTCTGCTTCACTCTCCCCATCTCCCCCCCCTCCCCACCCCCAATTTTG
TATTTATTTATTTTTTAATTATTTTGTGCAGCGATGGGGGGGGGGGGGGGGGGGGGGGGCGCGCCAGGCGGGGGGGGGCGGGGCGAGG
GGCGGGGCGGGGCGAGGCGGAGAGGTGCGGCGGCAGCCAATCAGAGCGGCGCGCTCCGAAAGTTTCCTTTTATGGCGAGGCGGCGGCG
GCGGCGGCCCTATAAAAAGCGAAGCGCGCGGCGGGCGGGAGTCGCTGCGCGCTGCCTTCGCCCCGTGCCCCGCTCCGCCGCCGCCTCG
CGCCGCCCGCCCCGGCTCTGACTGACCGCGTTACTCCCACAGGTGAGCGGGCGGGACGGCCCTTCTCCTCCGGGCTGTAATTAGCTGA
GCAAGAGGTAAGGGTTTAAGGGATGGTTGGTTGGTGGGGTATTAATGTTTAATTACCTGGAGCACCTGCCTGAAATCACTTTTTTTCA
GGTTGG

Preferably, therefore, the promoter sequence in the first and/or second expression vector comprises a nucleotide sequence substantially as set out in SEQ ID No: 8, or a fragment or variant thereof.

In another embodiment, the promoter in the first and/or second expression vector may be a human synapsin promoter. Either or both promoters in the expression vectors may be the human synapsin promoter. Preferably, the promoter is a human synapsin 1 promoter, which comprises 469 nucleotides. One embodiment of the nucleotide sequence forming the human synapsin I (SYN I) promoter is referred to herein as SEQ ID NO: 9, as follows:

[SEQ ID NO: 9]
CTGCAGAGGGCCCTGCGTATGAGTGCAAGTGGGTTTTAGGACCAGGATGAGGCGGGGTGGGGGTGCCTACCTGACGACCGACCCCGAC
CCACTGGACAAGCACCCAACCCCCATTCCCCAAATTGCGCATCCCCTATCAGAGAGGGGGAGGGGAAACAGGATGCGGCGAGGCGCGT
GCGCACTGCCAGCTTCAGCACCGCGGACAGTGCCTTCGCCCCCGCCTGGCGGCGCGCGCCACCGCCGCCTCAGCACTGAAGGCGCGCT
GACGTCACTCGCCGGTCCCCCGCAAACTCCCCTTCCCGGCCACCTTGGTCGCGTCCGCGCCGCCGCCGGCCCAGCCGGACCGCACCAC
GCGAGGCGCGAGATAGGGGGGCACGGGCGCGACCATCTGCGCTGCGGCGCCGGCGACTCAGCGCTGCCTCAGTCTGCGGTGGGCAGCG
GAGGAGTCGTGTCGTGCCTGAGAGCGCAG

The promoter may comprise a nucleotide sequence substantially as set out in SEQ ID No: 9, or a fragment or variant thereof.

In another embodiment, the promoter in the first and/or second expression vector is the chicken beta actin promoter with a cytomegalovirus enhancer (CB7). Either or both promoters in the expression vectors may comprise the chicken beta actin promoter with a cytomegalovirus enhancer. One embodiment of this promoter is referred to herein as SEQ ID NO: 10, as follows:

[SEQ ID No: 10]
gacattgattattgactagttattaatagtaatcaattacggggtcattagttcatagcccatatatggagttccgcgttacataact
tacggtaaatggcccgcctggctgaccgcccaacgacccccgcccattgacgtcaataatgacgtatgttcccatagtaacgccaata
gggactttccattgacgtcaatgggtggagtatttacggtaaactgcccacttggcagtacatcaagtgtatcatatgccaagtacgc
cccctattgacgtcaatgacggtaaatggcccgcctggcattatgcccagtacatgaccttatgggactttcctacttggcagtacat
ctacgtattagtcatcgctattaccatggtcgaggtgagccccacgttctgcttcactctccccatctcccccccctccccaccccca
attttgtatttatttattttttaattattttgtgcagcgatgggggcggggggggggggggggcgcgcgccaggcggggcggggcggg
gcgaggggcggggcggggcgaggcggagaggtgcggcggcagccaatcagagcggcgcgctccgaaagtttccttttatggcgaggcg
gcggcggcggcggccctataaaaagcgaagcgcgcgggggcg

The promoter may comprise a nucleotide sequence substantially as set out in SEQ ID No: 10, or a fragment or variant thereof.

In an embodiment, the promoter in the first and/or second expression vector is a Tetracycline-responsive element (TRE) promoter. Either or both promoters in the expression vectors may be the TRE promoter, one embodiment of which is referred to herein as SEQ ID NO: 11, as follows:

[SEQ ID No: 11]
ACGCGTGGAGCTAGTTATTAATAGTAATCAATTACGGGGTCATTAGTTCA
TAGCCCATATATGGAGTTCCGCGTTACATAACTTACGGTAAATGGCCCGC
CTGGCTGACCGCCCAACGACCCCCGCCCATTGACGTCAATAATGACGTAT
GTTCCCATAGTAACGTCAATAGGGACTTTCCATTGACGTCAATGGGTGGA
GTATTTACGGTAAACTGCCCACTTGGCAGTACATCAAGTGTATCATATGC
CAAGTACGCCCCCTATTGACGTCAATGACGGTAAATGGCCCGCCTGGCAT
TATGCCCAGTACATGACCTTATGGGACTTTCCTACTTGGCAGTACATCTA
CGTATTAGTCATCGCTATTACCATGGTGATGCGGTTTTGGCAGTACATCA
ATGGGCGTGGATAGCGGTTTGACTCACGGGGATTTCCAAGTCTCCACCCC
ATTGACGTCAATGGGAGTTTGTTTTGCACCAAAATCAACGGGACTTTCCA
AAATGTCGTAACAACTCCGCCCCATTGACGCAAATGGGCGGTAGGCGTGT
ACGGTGGGAGGTCTATATAAGCAGAGCTCGTTTAGTGAACCGTCAGATCG
CCTGGAGACGCCATCCACGCTGTTTTGACCTCCATAGAAGACACCGGGAC
CGATCCAGCCTCC

The promoter may comprise a nucleotide sequence substantially as set out in SEQ ID No: 11, or a fragment or variant thereof.

The promoter in the first and/or the second expression vector may not be the CMV promoter, or the CMV enhancer/promoter. However, in some embodiments, the promoter in the first and/or second expression vector is a CMV promoter. Either or both promoters in the expression vectors may be the CMV promoter, one embodiment of which is referred to herein as SEQ ID NO: 25, as follows:

[SEQ ID No: 25]
TAGTTATTAATAGTAATCAATTACGGGGTCATTAGTTCATAGCCCATATA
TGGAGTTCCGCGTTACATAACTTACGGTAAATGGCCCGCCTGGCTGACCG
CCCAACGACCCCCGCCCATTGACGTCAATAATGACGTATGTTCCCATAGT
AACGCCAATAGGGACTTTCCATTGACGTCAATGGGTGGAGTATTTACGGT
AAACTGCCCACTTGGCAGTACATCAAGTGTATCATATGCCAAGTACGCCC
CCTATTGACGTCAATGACGGTAAATGGCCCGCCTGGCATTATGCCCAGTA
CATGACCTTATGGGACTTTCCTACTTGGCAGTACATCTACGTATTAGTCA
TCGCTATTACCATGGTGATGCGGTTTTGGCAGTACATCAATGGGCGTGGA
TAGCGGTTTGACTCACGGGGATTTCCAAGTCTCCACCCCATTGACGTCAA
TGGGAGTTTGTTTTGGCACCAAAATCAACGGGACTTTCCAAAATGTCGTA
ACAACTCCGCCCCATTGACGCAAATGGGCGGTAGGCGTGTACGGTGGGAG
GTCTATATAAGCAGAGCTGGTTTAGTGAACCGTCAGATC

The promoter may comprise a nucleotide sequence substantially as set out in SEQ ID No: 25, or a fragment or variant thereof.

Therefore, the promoter in the first and/or second expression vector may comprise a nucleotide sequence substantially as set out in SEQ ID No: 8, 9, 10, 11, or 25 or a fragment or variant thereof. However, most preferably, the promoter may comprise a nucleotide sequence substantially as set out in SEQ ID No: 8, or a fragment or variant thereof, i.e. the CBh promoter. Preferably, the first and second vectors comprise the CBh promoter.

In some embodiments, the first expression vector comprises an intron disposed between its promoter and the nucleotide encoding TH. In some embodiments, the second expression vector comprises an intron disposed between its promoter and the nucleotide encoding GCH1.

Introns, as non-coding DNA sequences, have the function to regulation gene expression in eukaryotes by a variety of mechanisms, such as enhancing RNA polymerase II process activity, promoting the interaction between splicing proteins and certain transcription factors, connecting and facilitating multiple types of RNA processing mechanisms and affecting nuclear mRNA export, translational efficiency and RNA decay.

The intron may be at least 25, 50, 75, or 100 nucleotides in length. The intron may be at least 125, 150, 175, or 200 nucleotides in length. The intron may be at least 225, 250, 275, or 300 nucleotides in length.

The intron may be selected from a group of introns including: the human growth hormone (hGH) intron; the beta-actin intron; the minute virus of mouse (MVM) intron; the SV40 intron; and the EF-1 alpha intron.

The intron may be the human growth hormone (hGH) intron. The nucleotide sequence of the hGH intron is referred to herein as SEQ ID NO: 26, as follows:

[SEQ ID No: 26]
TTCGAACAGGTAAGCGCCCCTAAAATCCCTTTGGGCACAATGTGTCCTGA
GGGGAGAGGCAGCGACCTGTAGATGGGACGGGGGCACTAACCCTCAGGTT
TGGGGCTTCTGAATGTGAGTATCGCCATGTAAGCCCAGTATTTGGCCAAT
CTCAGAAAGCTCCTGGTCCCTGGAGGGATGGAGAGAGAAAAACAAACAGC
TCCTGGAGCAGGGAGAGTGCTGGCCTCTTGCTCTCCGGCTCCCTCTGTTG
CCCTCTGGTTTCTCCCCAGGTT

Hence, the first and/or second expression vector may comprise an intron which comprises a nucleotide sequence substantially as set out in SEQ ID No: 26, or a fragment or variant thereof.

Preferably, the intron is the beta-actin intron and/or the minute virus of mouse (MVM) intron. Preferably, the intron is the MVM intron. The nucleotide sequence of the MVM intron is referred to herein as SEQ ID NO: 27, as follows:

[SEQ ID No: 27]
GTAAGGGTTTAAGGGATGGTTGGTTGGTGGGGTATTAATGTTTAATTACC
TGGAGCACCTGCCTGAAATCACTTTTTTTCAG

Hence, the first and/or second expression vector may comprise an intron which comprises a nucleotide sequence substantially as set out in SEQ ID No: 27, or a fragment or variant thereof.

As discussed above, the advantage of using the CBh promoter is that it comprises both the beta-actin intron and the (MVM) intron, which is believed to contribute to improved expression levels of the TH (preferably the truncated TH) and GCH1.

Thus, the first and/or second expression vector may comprise a SYN1 promoter followed by an intron, which may be the MVM intron (SEQ ID No: 27) or the human growth hormone (hGH) intron (SEQ ID No: 26).

Alternatively, the first and/or second expression vector may comprise a chicken beta actin promoter with a cytomegalovirus enhancer (CB7) followed by an intron, which may be the MVM intron or the human growth hormone (hGH) intron.

Alternatively, the first and/or second expression vector may comprise a Tetracycline-responsive element (TRE) promoter followed by an intron, which may be the MVM intron or the human growth hormone (hGH) intron.

Alternatively, the first and/or second expression vector may comprise a CMV promoter followed by an intron, which may be the MVM intron or the human growth hormone (hGH) intron.

In an embodiment, the first and/or second expression vector may further comprise a nucleotide sequence encoding Woodchuck Hepatitis Virus Post-transcriptional Regulatory Element (WPRE), which further enhances the expression of TH1 and/or GCH1, respectively. In some embodiments, the first expression vector does not comprise a nucleotide sequence encoding a WPRE.

Preferably, the second expression vector further comprises a nucleotide sequence encoding Woodchuck Hepatitis Virus Post-transcriptional Regulatory Element (WPRE).

Preferably, the WPRE coding sequence is disposed 3′ of GCH1 coding sequence on the second expression vector.

One embodiment of the WPRE is 592 bp long, including gamma-alpha-beta elements, and is referred to herein as SEQ ID No: 12, as follows:

[SEQ ID NO: 12]
AATCAACCTCTGGATTACAAAATTTGTGAAAGATTGACTGGTATTCTTAA
CTATGTTGCTCCTTTTACGCTATGTGGATACGCTGCTTTAATGCCTTTGT
ATCATGCTATTGCTTCCCGTATGGCTTTCATTTTCTCCTCCTTGTATAAA
TCCTGGTTGCTGTCTCTTTATGAGGAGTTGTGGCCCGTTGTCAGGCAACG
TGGCGTGGTGTGCACTGTGTTTGCTGACGCAACCCCCACTGGTTGGGGCA
TTGCCACCACCTGTCAGCTCCTTTCCGGGACTTTCGCTTTCCCCCTCCCT
ATTGCCACGGCGGAACTCATCGCCGCCTGCCTTGCCCGCTGCTGGACAGG
GGCTCGGCTGTTGGGCACTGACAATTCCGTGGTGTTGTCGGGGAAGCTGA
CGTCCTTTCCATGGCTGCTCGCCTGTGTTGCCACCTGGATTCTGCGCGGG
ACGTCCTTCTGCTACGTCCCTTCGGCCCTCAATCCAGCGGACCTTCCTTC
CCGCGGCCTGCTGCCGGCTCTGCGGCCTCTTCCGCGTCTTCGCCTTCGCC
CTCAGACGAGTCGGATCTCCCTTTGGGCCGCCTCCCCGCCTG

Preferably, the WPRE comprises a nucleic acid sequence substantially as set out in SEQ ID No: 11, or a fragment or variant thereof.

However, in a preferred embodiment, a truncated WPRE is used, which is 247 bp long due to deletion of the beta element, and which is referred to herein as SEQ ID No: 13, as follows:

[SEQ ID NO: 13]
AATCAACCTCTGGATTACAAAATTTGTGAAAGATTGACTGGTATTCTTAA
CTATGTTGCTCCTTTTACGCTATGTGGATACGCTGCTTTAATGCCTTTGT
ATCATGCTATTGCTTCCCGTATGGCTTTCATTTTCTCCTCCTTGTATAAA
TCCTGGTTAGTTCTTGCCACGGCGGAACTCATCGCCGCCTGCCTTGCCCG
CTGCTGGACAGGGGCTCGGCTGTTGGGCACTGACAATTCCGTGGTGT

Preferably, the WPRE comprises a nucleic acid sequence substantially as set out in SEQ ID No: 13, or a fragment or variant thereof.

Preferably, the first expression vector comprises a nucleotide sequence encoding a polyA tail. Preferably, the second expression vector comprises a nucleotide sequence encoding a polyA tail. Preferably, the polyA tail coding sequence is disposed 3′ of the TH and/or GCH1 coding sequence, and preferably 3′ of the WPRE coding sequence, if present in the second vector.

In one embodiment, the polyA tail comprises the simian virus 40 poly-A 224 bp sequence (i.e. SV40 polyA tail). One embodiment of the SV40 polyA tail is referred to herein as SEQ ID No: 14, as follows:

[SEQ ID NO: 14]
AGCAGACATGATAAGATACATTGATGAGTTTGGACAAACCACAACTAGAA
TGCAGTGAAAAAAATGCTTTATTTGTGAAATTTGTGATGCTATTGCTTTA
TTTGTAACCATTATAAGCTGCAATAAACAAGTTAACAACAACAATTGCAT
TCATTTTATGTTTCAGGTTCAGGGGGAGGTGTGGGAGGTTTTTTAAAGCA
AGTAAAACCTCTACAAATGTGGTA

Preferably, the polyA tail comprises a nucleic acid sequence substantially as set out in SEQ ID No: 14, or a fragment or variant thereof.

In another embodiment, the polyA tail comprises the bovine growth hormone (BGH) poly A 208 bp sequence. One embodiment of the BGH polyA tail is referred to herein as SEQ ID No: 15, as follows:

[SEQ ID No: 15]
CTGTGCCTTCTAGTTGCCAGCCATCTGTTGTTTGCCCCTCCCCCGTGCCT
TCCTTGACCCTGGAAGGTGCCACTCCCACTGTCCTTTCCTAATAAAATGA
GGAAATTGCATCGCATTGTCTGAGTAGGTGTCATTCTATTCTGGGGGGTG
GGGTGGGGCAGGACAGCAAGGGGGAGGATTGGGAAGAGAATAGCAGGCAT
GCTGGGGA

Preferably, the polyA tail comprises a nucleic acid sequence substantially as set out in SEQ ID No: 15, or a fragment or variant thereof.

Preferably, the first expression vector comprises a left and/or a right Inverted Terminal Repeat sequences (ITRs). Preferably, the second expression vector comprises a left and/or a right Inverted Terminal Repeat sequences (ITRs). Preferably, each ITR is disposed at the 5′ and/or 3′ end of the expression vector.

The preferred self-complementary first expression vector comprises one Inverted Terminal Repeat (ITR) sequence and one modified ITR sequence in which the terminal resolution site is deleted. The preferred self-complementary second expression vector comprises one Inverted Terminal Repeat (ITR) sequence and one modified ITR sequence in which the terminal resolution site is deleted. One embodiment of the ITR in which the terminal resolution site is deleted is referred to herein as SEQ ID No:16:

[SEQ ID No: 16]
CCACTCCCTCTCTGCGCGCTCGCTCGCTCACTGAGGCCGGGCGACCAAAG
GTCGCCCGACGCCCGGGCTTTGCCCGGGCGGCCTCAGTGAGCGAGCGAGC
GCGCAGAGAGGGA

Thus, preferably the first and/or second expression vector comprises an ITR comprising a nucleic acid sequence substantially as set out in SEQ ID No: 16, or a fragment or variant thereof.

The first expression vector may comprise a nucleotide sequence encoding a 3′ untranslated region (3′ UTR). The second expression vector may comprise a nucleotide sequence encoding a 3′ untranslated region (3′ UTR). Preferably, the 3′ UTR coding sequence is disposed 3′ of the TH and/or GCH1 coding sequence, and preferably 5′ of the poly A tail if present and/or 5′ of the WPRE coding sequence, if present.

One embodiment of the 3′ UTR coding sequence in the first expression vector (i.e. a 3′ UTR of TH) is referred to herein as SEQ ID No: 28:

[SEQ ID No: 28]
GTGCACGGCGTCCCTGAGGGCCCTTCCCAACCTCCCCTGGTCCTGCACTG
TCCCGGAGCTCAGGCCCTGGTGAGGGGCTGGGTCCCGGGTGCCCCCCATG
CCCTCCCTGCTGCCAGGCTCCCACTGCCCCTGCACCTGCTTCTCAGCGCA
ACAGCTGTGTGTGCCCGTGGTGAGGTTGTGCTGCCTGTGGTGAGGTCCTG
TCCTGGCTCCCAGGGTCCTGGGGGCTGCTGCACTGCCCTCCGCCCTTCCC
TGACACTGTCTGCTGCCCCAATCACCGTCACAATAAAAGAAACTGTGGTC
TCTA

Thus, preferably the first expression vector comprises a 3′ UTR coding sequence comprising a nucleic acid sequence substantially as set out in SEQ ID No: 28, or a fragment or variant thereof.

The first expression vector may comprise a nucleotide sequence encoding a 5′ untranslated region (5′ UTR). The second expression vector may comprise a nucleotide sequence encoding a 5′ untranslated region (5′ UTR). Preferably, the 5′ UTR coding sequence is disposed 5′ of the TH and/or GCH1 coding sequence, and preferably 3′ of the promoter.

The 5′ UTR coding sequence is preferably a Kozak sequence. One embodiment of the 5′ UTR coding sequence in the first and/or second expression vector is referred to herein as SEQ ID No: 29:

[SEQ ID No: 29]
GCCACC

Thus, preferably the first and/or second expression vector comprises a 5′ UTR coding sequence comprising a nucleic acid sequence substantially as set out in SEQ ID No: 29, or a fragment or variant thereof.

In an embodiment, the first expression vector may comprise, in this specified order, a 5′ promoter; a sequence encoding TH; and a 3′ sequence encoding a poly A tail. In an embodiment, the second expression vector may comprise, in this specified order, a 5′ promoter; a sequence encoding GCH1; and a 3′ sequence encoding a poly A tail. The use of 5′ and 3′ described herein indicates that the features are either upstream or downstream, and is not intended to indicate that the features are necessarily terminal features.

In an embodiment, the first expression vector may comprise, in this specified order, a 5′ ITR; a promoter; a sequence encoding human truncated TH; a sequence encoding a poly A tail; and a 3′ ITR. In an embodiment, the second expression vector may comprise, in this specified order, a 5′ ITR; a promoter; a sequence encoding human GCH1; a sequence encoding a poly A tail; and a 3′ ITR.

In an embodiment, the first expression vector may comprise, in this specified order, a 5′ ITR; a promoter; an intron; a sequence encoding human truncated TH; a sequence encoding a poly A tail; and a 3′ ITR. In an embodiment, the second expression vector may comprise, in this specified order, a 5′ ITR; a promoter; an intron; a sequence encoding human GCH1; a sequence encoding a polyA tail; and a 3′ ITR.

In an embodiment, the first expression vector may comprise, in this specified order, a 5′ ITR; a CBh promoter; a sequence encoding human truncated TH; a sequence encoding a poly A tail; and a 3′ ITR. In another embodiment, the second expression vector may comprise, in this specified order, a 5′ ITR; a CBh promoter; a sequence encoding GCH1; a sequence encoding WPRE; a sequence encoding a poly A tail; and a 3′ ITR.

In a preferred embodiment, the first expression vector may comprise, in this specified order, a 5′ ITR; a CBh promoter; a sequence encoding human truncated TH; a sequence encoding a poly A tail; and a 3′ ITR, in which the terminal resolution site is deleted. In a preferred embodiment, the second expression vector may comprise, in this specified order, 5′ ITR; a CBh promoter; a sequence encoding GCH1; a sequence encoding WPRE; a sequence encoding a poly A tail; and a 3′ ITR, in which the terminal resolution site is deleted. Preferably, the composition of the first aspect comprises the first and second expression vectors as described herein.

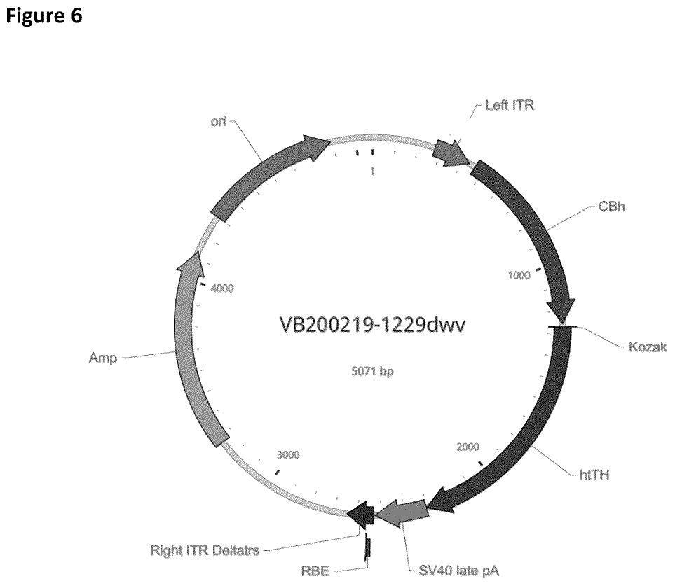

The following sequence, referred to herein as SEQ ID No: 17 and shown in FIG. 6, depicts a vector comprising a CBh promoter operably linked to an htTH (human truncated TH) coding sequence:

[SEQ ID No: 17]
AGCGCCCAATACGCAAACCGCCTCTCCCCGCGCGTTGGCCGATTCATTAATGCAGCTGGCACGACAGGTTTCCCGACT
GGAAAGCGGGCAGTGAGCGCAACGCAATTAATGTGAGTTAGCTCACTCATTAGGCACCCCAGGCTTTACACTTTATGC
TTCCGGCTCGTATGTTGTGTGGAATTGTGAGCGGATAACAATTTCACACAGGAAACAGCTATGACCATGATTACGCCA
AGCTCTCGAGATCTAGAAAGCTTCCCGGGGGGATCTGGGCCACTCCCTCTCTGCGCGCTCGCTCGCTCACTGAGGCCG
GGCGACCAAAGGTCGCCCGACGCCCGGGCTTTGCCCGGGCGGCCTCAGTGAGCGAGCGAGCGCGCAGAGAGGGAGTGG
CCAACTCCATCACTAGGGGTTCCTGGAGGGGTGGAGTCGTGACCTAGGCAACTTTGTATAGAAAAGTTGCGTTACATA
ACTTACGGTAAATGGCCCGCCTGGCTGACCGCCCAACGACCCCCGCCCATTGACGTCAATAGTAACGCCAATAGGGAC
TTTCCATTGACGTCAATGGGTGGAGTATTTACGGTAAACTGCCCACTTGGCAGTACATCAAGTGTATCATATGCCAAG
TACGCCCCCTATTGACGTCAATGACGGTAAATGGCCCGCCTGGCATTGTGCCCAGTACATGACCTTATGGGACTTTCC
TACTTGGCAGTACATCTACGTATTAGTCATCGCTATTACCATGGTCGAGGTGAGCCCCACGTTCTGCTTCACTCTCCC
CATCTCCCCCCCCTCCCCACCCCCAATTTTGTATTTATTTATTTTTTAATTATTTTGTGCAGCGATGGGGGCGGGGGG
GGGGGGGGGGCGCGCGCCAGGCGGGGCGGGGCGGGGCGAGGGGCGGGGCGGGGCGAGGCGGAGAGGTGCGGCGGCAGC
CAATCAGAGCGGCGCGCTCCGAAAGTTTCCTTTTATGGCGAGGCGGCGGCGGCGGCGGCCCTATAAAAAGCGAAGCGC
GCGGCGGGCGGGAGTCGCTGCGCGCTGCCTTCGCCCCGTGCCCCGCTCCGCCGCCGCCTCGCGCCGCCCGCCCCGGCT
CTGACTGACCGCGTTACTCCCACAGGTGAGCGGGCGGGACGGCCCTTCTCCTCCGGGCTGTAATTAGCTGAGCAAGAG
GTAAGGGTTTAAGGGATGGTTGGTTGGTGGGGTATTAATGTTTAATTACCTGGAGCACCTGCCTGAAATCACTTTTTT
TCAGGTTGGCAAGTTTGTACAGCCACCATGAGCCCCGCGGGGCCCAAGGTCCCCTGGTTCCCAAGAAAAGTGTCAGAG
CTGGACAAGTGTCATCACCTGGTCACCAAGTTCGACCCTGACCTGGACTTGGACCACCCGGGCTTCTCGGACCAGGTG
TACCGCCAGCGCAGGAAGCTGATTGCTGAGATCGCCTTCCAGTACAGGCACGGCGACCCGATTCCCCGTGTGGAGTAC
ACCGCCGAGGAGATTGCCACCTGGAAGGAGGTCTACACCACGCTGAAGGGCCTCTACGCCACGCACGCCTGCGGGGAG
CACCTGGAGGCCTTTGCTTTGCTGGAGCGCTTCAGCGGCTACCGGGAAGACAATATCCCCCAGCTGGAGGACGTCTCC
CGCTTCCTGAAGGAGCGCACGGGCTTCCAGCTGCGGCCTGTGGCCGGCCTGCTGTCCGCCCGGGACTTCCTGGCCAGC
CTGGCCTTCCGCGTGTTCCAGTGCACCCAGTATATCCGCCACGCGTCCTCGCCCATGCACTCCCCTGAGCCGGACTGC
TGCCACGAGCTGCTGGGGCACGTGCCCATGCTGGCCGACCGCACCTTCGCGCAGTTCTCGCAGGACATTGGCCTGGCG
TCCCTGGGGGCCTCGGATGAGGAAATTGAGAAGCTGTCCACGCTGTACTGGTTCACGGTGGAGTTCGGGCTGTGTAAG
CAGAACGGGGAGGTGAAGGCCTATGGTGCCGGGCTGCTGTCCTCCTACGGGGAGCTCCTGCACTGCCTGTCTGAGGAG
CCTGAGATTCGGGCCTTCGACCCTGAGGCTGCGGCCGTGCAGCCCTACCAAGACCAGACGTACCAGTCAGTCTACTTC
GTGTCTGAGAGCTTCAGTGACGCCAAGGACAAGCTCAGGAGCTATGCCTCACGCATCCAGCGCCCCTTCTCCGTGAAG
TTCGACCCGTACACGCTGGCCATCGACGTGCTGGACAGCCCCCAGGCCGTGCGGCGCTCCCTGGAGGGTGTCCAGGAT
GAGCTGGACACCCTTGCCCATGCGCTGAGTGCCATTGGCTAACAGACATGATAAGATACATTGATGAGTTTGGACAAA
CCACAACTAGAATGCAGTGAAAAAAATGCTTTATTTGTGAAATTTGTGATGCTATTGCTTTATTTGTAACCATTATAA
GCTGCAATAAACAAGTTAACAACAACAATTGCATTCATTTTATGTTTCAGGTTCAGGGGGAGGTGTGGGAGGTTTTTT
AAAGCAAGTAAAACCTCTACAAATGTGGTAACTAGTCCACTCCCTCTCTGCGCGCTCGCTCGCTCACTGAGGCCGGGC
GACCAAAGGTCGCCCGACGCCCGGGCTTTGCCCGGGCGGCCTCAGTGAGCGAGCGAGCGCGCAGAGAGGGACAGATCC
GGGCCCGCATGCGTCGACAATTCACTGGCCGTCGTTTTACAACGTCGTGACTGGGAAAACCCTGGCGTTACCCAACTT
AATCGCCTTGCAGCACATCCCCCTTTCGCCAGCTGGCGTAATAGCGAAGAGGCCCGCACCGATCGCCCTTCCCAACAG
TTGCGCAGCCTGAATGGCGAATGGCGCCTGATGCGGTATTTTCTCCTTACGCATCTGTGCGGTATTTCACACCGCATA
TGGTGCACTCTCAGTACAATCTGCTCTGATGCCGCATAGTTAAGCCAGCCCCGACACCCGCCAACACCCGCTGACGCG
CCCTGACGGGCTTGTCTGCTCCCGGCATCCGCTTACAGACAAGCTGTGACCGTCTCCGGGAGCTGCATGTGTCAGAGG
TTTTCACCGTCATCACCGAAACGCGCGAGACGAAAGGGCCTCGTGATACGCCTATTTTTATAGGTTAATGTCATGATA
ATAATGGTTTCTTAGACGTCAGGTGGCACTTTTCGGGGAAATGTGCGCGGAACCCCTATTTGTTTATTTTTCTAAATA
CATTCAAATATGTATCCGCTCATGAGACAATAACCCTGATAAATGCTTCAATAATATTGAAAAAGGAAGAGTATGAGT
ATTCAACATTTCCGTGTCGCCCTTATTCCCTTTTTTGCGGCATTTTGCCTTCCTGTTTTTGCTCACCCAGAAACGCTG
GTGAAAGTAAAAGATGCTGAAGATCAGTTGGGTGCACGAGTGGGTTACATCGAACTGGATCTCAACAGCGGTAAGATC
CTTGAGAGTTTTCGCCCCGAAGAACGTTTTCCAATGATGAGCACTTTTAAAGTTCTGCTATGTGGCGCGGTATTATCC
CGTATTGACGCCGGGCAAGAGCAACTCGGTCGCCGCATACACTATTCTCAGAATGACTTGGTTGAGTACTCACCAGTC
ACAGAAAAGCATCTTACGGATGGCATGACAGTAAGAGAATTATGCAGTGCTGCCATAACCATGAGTGATAACACTGCG
GCCAACTTACTTCTGACAACGATCGGAGGACCGAAGGAGCTAACCGCTTTTTTGCACAACATGGGGGATCATGTAACT
CGCCTTGATCGTTGGGAACCGGAGCTGAATGAAGCCATACCAAACGACGAGCGTGACACCACGATGCCTGTAGCAATG
GCAACAACGTTGCGCAAACTATTAACTGGCGAACTACTTACTCTAGCTTCCCGGCAACAATTAATAGACTGGATGGAG
GCGGATAAAGTTGCAGGACCACTTCTGCGCTCGGCCCTTCCGGCTGGCTGGTTTATTGCTGATAAATCTGGAGCCGGT
GAGCGTGGGTCTCGCGGTATCATTGCAGCACTGGGGCCAGATGGTAAGCCCTCCCGTATCGTAGTTATCTACACGACG
GGGAGTCAGGCAACTATGGATGAACGAAATAGACAGATCGCTGAGATAGGTGCCTCACTGATTAAGCATTGGTAACTG
TCAGACCAAGTTTACTCATATATACTTTAGATTGATTTAAAACTTCATTTTTAATTTAAAAGGATCTAGGTGAAGATC
CTTTTTGATAATCTCATGACCAAAATCCCTTAACGTGAGTTTTCGTTCCACTGAGCGTCAGACCCCGTAGAAAAGATC
AAAGGATCTTCTTGAGATCCTTTTTTTCTGCGCGTAATCTGCTGCTTGCAAACAAAAAAACCACCGCTACCAGCGGTG
GTTTGTTTGCCGGATCAAGAGCTACCAACTCTTTTTCCGAAGGTAACTGGCTTCAGCAGAGCGCAGATACCAAATACT
GTTCTTCTAGTGTAGCCGTAGTTAGGCCACCACTTCAAGAACTCTGTAGCACCGCCTACATACCTCGCTCTGCTAATC
CTGTTACCAGTGGCTGCTGCCAGTGGCGATAAGTCGTGTCTTACCGGGTTGGACTCAAGACGATAGTTACCGGATAAG
GCGCAGCGGTCGGGCTGAACGGGGGGTTCGTGCACACAGCCCAGCTTGGAGCGAACGACCTACACCGAACTGAGATAC
CTACAGCGTGAGCTATGAGAAAGCGCCACGCTTCCCGAAGGGAGAAAGGCGGACAGGTATCCGGTAAGCGGCAGGGTC
GGAACAGGAGAGCGCACGAGGGAGCTTCCAGGGGGAAACGCCTGGTATCTTTATAGTCCTGTCGGGTTTCGCCACCTC
TGACTTGAGCGTCGATTTTTGTGATGCTCGTCAGGGGGGCGGAGCCTATGGAAAAACGCCAGCAACGCGGCCTTTTTA
CGGTTCCTGGCCTTTTGCTGGCCTTTTGCTCACATGTTCTTTCCTGCGTTATCCCCTGATTCTGTGGATAACCGTATT
ACCGCCTTTGAGTGAGCTGATACCGCTCGCCGCAGCCGAACGACCGAGCGCAGCGAGTCAGTGAGCGAGGAAGCGGAA
G

Preferably, the first expression vector comprises a nucleic acid sequence substantially as set out in SEQ ID No: 17, or a fragment or variant thereof.

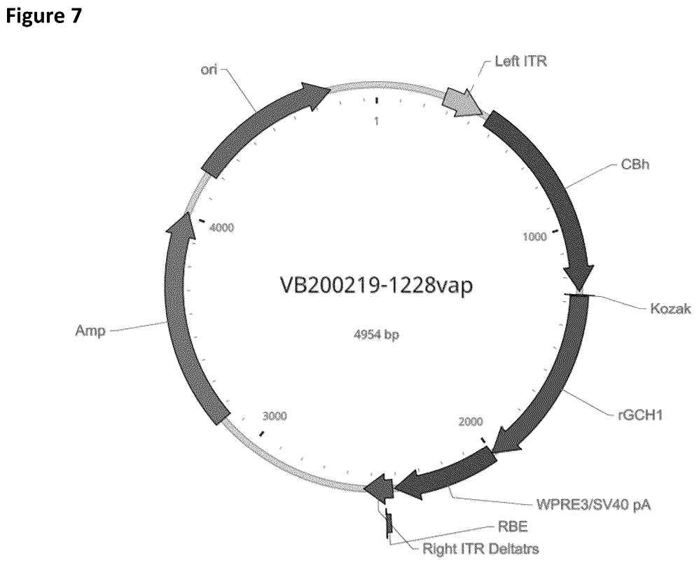

The following sequence, referred to herein as SEQ ID No: 18 and shown in FIG. 7, depicts a vector comprising a CBh promoter operably linked to a GCH-1 coding sequence:

[SEQ ID No: 18]
AGCGCCCAATACGCAAACCGCCTCTCCCCGCGCGTTGGCCGATTCATTAATGCAGCTGGCACGACAGGTTTCCCGACT
GGAAAGCGGGCAGTGAGCGCAACGCAATTAATGTGAGTTAGCTCACTCATTAGGCACCCCAGGCTTTACACTTTATGC
TTCCGGCTCGTATGTTGTGTGGAATTGTGAGCGGATAACAATTTCACACAGGAAACAGCTATGACCATGATTACGCCA
AGCTCTCGAGATCTAGAAAGCTTCCCGGGGGGATCTGGGCCACTCCCTCTCTGCGCGCTCGCTCGCTCACTGAGGCCG
GGCGACCAAAGGTCGCCCGACGCCCGGGCTTTGCCCGGGCGGCCTCAGTGAGCGAGCGAGCGCGCAGAGAGGGAGTGG
CCAACTCCATCACTAGGGGTTCCTGGAGGGGTGGAGTCGTGACCTAGGCAACTTTGTATAGAAAAGTTGCGTTACATA
ACTTACGGTAAATGGCCCGCCTGGCTGACCGCCCAACGACCCCCGCCCATTGACGTCAATAGTAACGCCAATAGGGAC
TTTCCATTGACGTCAATGGGTGGAGTATTTACGGTAAACTGCCCACTTGGCAGTACATCAAGTGTATCATATGCCAAG
TACGCCCCCTATTGACGTCAATGACGGTAAATGGCCCGCCTGGCATTGTGCCCAGTACATGACCTTATGGGACTTTCC
TACTTGGCAGTACATCTACGTATTAGTCATCGCTATTACCATGGTCGAGGTGAGCCCCACGTTCTGCTTCACTCTCCC
CATCTCCCCCCCCTCCCCACCCCCAATTTTGTATTTATTTATTTTTTAATTATTTTGTGCAGCGATGGGGGGGGGGGG
GGGGGGGGGGCGCGCGCCAGGCGGGGCGGGGCGGGGCGAGGGGCGGGGCGGGGCGAGGCGGAGAGGTGCGGCGGCAGC
CAATCAGAGCGGCGCGCTCCGAAAGTTTCCTTTTATGGCGAGGCGGCGGCGGCGGCGGCCCTATAAAAAGCGAAGCGC
GCGGCGGGCGGGAGTCGCTGCGCGCTGCCTTCGCCCCGTGCCCCGCTCCGCCGCCGCCTCGCGCCGCCCGCCCCGGCT
CTGACTGACCGCGTTACTCCCACAGGTGAGCGGGCGGGACGGCCCTTCTCCTCCGGGCTGTAATTAGCTGAGCAAGAG
GTAAGGGTTTAAGGGATGGTTGGTTGGTGGGGTATTAATGTTTAATTACCTGGAGCACCTGCCTGAAATCACTTTTTT
TCAGGTTGGCAAGTTTGTACAGCCACCATGGAGAAGCCGCGGGGTGTAAGGTGCACCAATGGGTTCCCCGAGCGGGAG
CTGCCGCGGCCCGGGGCCAGCCGACCTGCCGAGAAGTCCCGGCCGCCCGAGGCCAAGGGCGCACAGCCAGCCGACGCC
TGGAAGGCAGGGCGGCCCCGCAGCGAGGAGGATAACGAGCTGAACCTCCCCAACCTGGCGGCCGCTTACTCGTCCATC
CTGCGCTCGCTGGGCGAGGACCCCCAGCGGCAGGGGCTGCTCAAGACGCCCTGGAGGGCGGCCACCGCCATGCAGTTC
TTCACCAAGGGATACCAGGAGACCATCTCAGATGTCCTGAACGATGCTATATTTGATGAGGACCATGACGAGATGGTG
ATTGTGAAGGACATTGACATGTTTTCCATGTGTGAGCATCACCTGGTCCCATTTGTGGGAAGGGTCCATATTGGTTAT
CTTCCTAACAAGCAAGTCCTTGGTCTCAGCAAACTTGCCAGGATTGTGGAAATCTACAGTAGAAGACTACAAGTTCAA
GAACGCCTTACCAAACAGATTGCAGTGGCCATCACAGAAGCCTTGCAGCCTGCTGGCGTCGGGGTAGTGATTGAAGCA
ACACACATGTGTATGGTCATGCGAGGTGTGCAGAAAATGAACAGCAAGACTGTCACTAGCACCATGCTAGGCGTGTTC
CGGGAAGACCCAAAGACTCGGGAGGAGTTCCTCACACTCATCAGGAGCTGAAATCAACCTCTGGATTACAAAATTTGT
GAAAGATTGACTGGTATTCTTAACTATGTTGCTCCTTTTACGCTATGTGGATACGCTGCTTTAATGCCTTTGTATCAT
GCTATTGCTTCCCGTATGGCTTTCATTTTCTCCTCCTTGTATAAATCCTGGTTAGTTCTTGCCACGGCGGAACTCATC
GCCGCCTGCCTTGCCCGCTGCTGGACAGGGGCTCGGCTGTTGGGCACTGACAATTCCGTGGTGTTTATTTGTGAAATT
TGTGATGCTATTGCTTTATTTGTAACCATCTAGCTTTATTTGTGAAATTTGTGATGCTATTGCTTTATTTGTAACCAT
TATAAGCTGCAATAAACAAGTTAACAACAACAATTGCATTCATTTTATGTTTCAGGTTCAGGGGGAGAGACTAGTCCA
CTCCCTCTCTGCGCGCTCGCTCGCTCACTGAGGCCGGGCGACCAAAGGTCGCCCGACGCCCGGGCTTTGCCCGGGCGG
CCTCAGTGAGCGAGCGAGCGCGCAGAGAGGGACAGATCCGGGCCCGCATGCGTCGACAATTCACTGGCCGTCGTTTTA
CAACGTCGTGACTGGGAAAACCCTGGCGTTACCCAACTTAATCGCCTTGCAGCACATCCCCCTTTCGCCAGCTGGCGT
AATAGCGAAGAGGCCCGCACCGATCGCCCTTCCCAACAGTTGCGCAGCCTGAATGGCGAATGGCGCCTGATGCGGTAT
TTTCTCCTTACGCATCTGTGCGGTATTTCACACCGCATATGGTGCACTCTCAGTACAATCTGCTCTGATGCCGCATAG
TTAAGCCAGCCCCGACACCCGCCAACACCCGCTGACGCGCCCTGACGGGCTTGTCTGCTCCCGGCATCCGCTTACAGA
CAAGCTGTGACCGTCTCCGGGAGCTGCATGTGTCAGAGGTTTTCACCGTCATCACCGAAACGCGCGAGACGAAAGGGC
CTCGTGATACGCCTATTTTTATAGGTTAATGTCATGATAATAATGGTTTCTTAGACGTCAGGTGGCACTTTTCGGGGA
AATGTGCGCGGAACCCCTATTTGTTTATTTTTCTAAATACATTCAAATATGTATCCGCTCATGAGACAATAACCCTGA
TAAATGCTTCAATAATATTGAAAAAGGAAGAGTATGAGTATTCAACATTTCCGTGTCGCCCTTATTCCCTTTTTTGCG
GCATTTTGCCTTCCTGTTTTTGCTCACCCAGAAACGCTGGTGAAAGTAAAAGATGCTGAAGATCAGTTGGGTGCACGA
GTGGGTTACATCGAACTGGATCTCAACAGCGGTAAGATCCTTGAGAGTTTTCGCCCCGAAGAACGTTTTCCAATGATG
AGCACTTTTAAAGTTCTGCTATGTGGCGCGGTATTATCCCGTATTGACGCCGGGCAAGAGCAACTCGGTCGCCGCATA
CACTATTCTCAGAATGACTTGGTTGAGTACTCACCAGTCACAGAAAAGCATCTTACGGATGGCATGACAGTAAGAGAA
TTATGCAGTGCTGCCATAACCATGAGTGATAACACTGCGGCCAACTTACTTCTGACAACGATCGGAGGACCGAAGGAG
CTAACCGCTTTTTTGCACAACATGGGGGATCATGTAACTCGCCTTGATCGTTGGGAACCGGAGCTGAATGAAGCCATA
CCAAACGACGAGCGTGACACCACGATGCCTGTAGCAATGGCAACAACGTTGCGCAAACTATTAACTGGCGAACTACTT
ACTCTAGCTTCCCGGCAACAATTAATAGACTGGATGGAGGCGGATAAAGTTGCAGGACCACTTCTGCGCTCGGCCCTT
CCGGCTGGCTGGTTTATTGCTGATAAATCTGGAGCCGGTGAGCGTGGGTCTCGCGGTATCATTGCAGCACTGGGGCCA
GATGGTAAGCCCTCCCGTATCGTAGTTATCTACACGACGGGGAGTCAGGCAACTATGGATGAACGAAATAGACAGATC
GCTGAGATAGGTGCCTCACTGATTAAGCATTGGTAACTGTCAGACCAAGTTTACTCATATATACTTTAGATTGATTTA
AAACTTCATTTTTAATTTAAAAGGATCTAGGTGAAGATCCTTTTTGATAATCTCATGACCAAAATCCCTTAACGTGAG
TTTTCGTTCCACTGAGCGTCAGACCCCGTAGAAAAGATCAAAGGATCTTCTTGAGATCCTTTTTTTCTGCGCGTAATC
TGCTGCTTGCAAACAAAAAAACCACCGCTACCAGCGGTGGTTTGTTTGCCGGATCAAGAGCTACCAACTCTTTTTCCG
AAGGTAACTGGCTTCAGCAGAGCGCAGATACCAAATACTGTTCTTCTAGTGTAGCCGTAGTTAGGCCACCACTTCAAG
AACTCTGTAGCACCGCCTACATACCTCGCTCTGCTAATCCTGTTACCAGTGGCTGCTGCCAGTGGCGATAAGTCGTGT
CTTACCGGGTTGGACTCAAGACGATAGTTACCGGATAAGGCGCAGCGGTCGGGCTGAACGGGGGGTTCGTGCACACAG
CCCAGCTTGGAGCGAACGACCTACACCGAACTGAGATACCTACAGCGTGAGCTATGAGAAAGCGCCACGCTTCCCGAA
GGGAGAAAGGCGGACAGGTATCCGGTAAGCGGCAGGGTCGGAACAGGAGAGCGCACGAGGGAGCTTCCAGGGGGAAAC
GCCTGGTATCTTTATAGTCCTGTCGGGTTTCGCCACCTCTGACTTGAGCGTCGATTTTTGTGATGCTCGTCAGGGGGG
CGGAGCCTATGGAAAAACGCCAGCAACGCGGCCTTTTTACGGTTCCTGGCCTTTTGCTGGCCTTTTGCTCACATGTTC
TTTCCTGCGTTATCCCCTGATTCTGTGGATAACCGTATTACCGCCTTTGAGTGAGCTGATACCGCTCGCCGCAGCCGA
ACGACCGAGCGCAGCGAGTCAGTGAGCGAGGAAGCGGAAG

Preferably, the second expression vector comprises a nucleic acid sequence substantially as set out in SEQ ID No: 18, or a fragment or variant thereof.

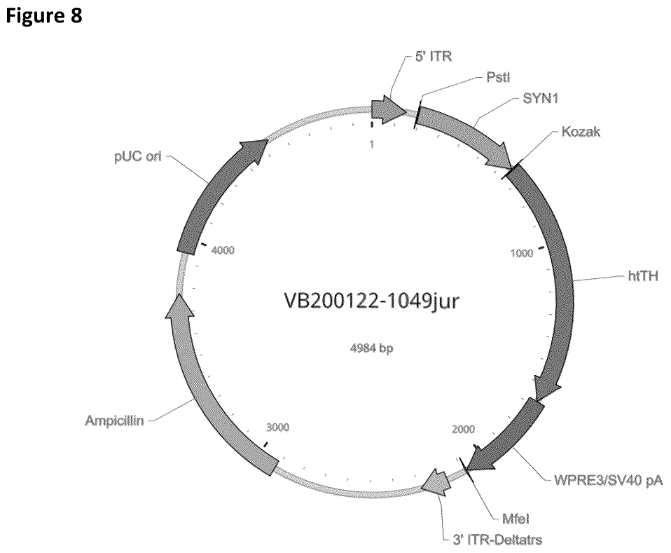

The following sequence, referred to herein as SEQ ID No: 19 and shown in FIG. 8, depicts a vector (i.e. the first expression vector of the composition of the first aspect) comprising a SYN1 promoter operably linked to a htTH coding sequence:

[SEQ ID No: 19]
GGCCACTCCCTCTCTGCGCGCTCGCTCGCTCACTGAGGCCGGGCGACCAAAGGTCGCCCGACGCCCGGGCTTTGCCCG
GGCGGCCTCAGTGAGCGAGCGAGCGCGCAGAGAGGGAGTGGCCAACTCCATCACTAGGGGTTCCTGGAGGGGTGGAGT
CGTGACCTAGGCAACTTTGTATAGAAAAGTTGCTGCAGAGTGCAAGTGGGTTTTAGGACCAGGATGAGGCGGGGTGGG
GGTGCCTACCTGACGACCGACCCCGACCCACTGGACAAGCACCCAACCCCCATTCCCCAAATTGCGCATCCCCTATCA
GAGAGGGGGAGGGGAAACAGGATGCGGCGAGGCGCGTGCGCACTGCCAGCTTCAGCACCGCGGACAGTGCCTTCGCCC
CCGCCTGGCGGCGCGCGCCACCGCCGCCTCAGCACTGAAGGCGCGCTGACGTCACTCGCCGGTCCCCCGCAAACTCCC
CTTCCCGGCCACCTTGGTCGCGTCCGCGCCGCCGCCGGCCCAGCCGGACCGCACCACGCGAGGCGCGAGATAGGGGGG
CACGGGCGCGACCATCTGCGCTGCGGCGCCGGCGACTCAGCGCTGCCTCAGTCTGCGGTGGGCAGCGGAGGAGTCGTG
TCGTGCCTGAGAGCGCAGGCCACCATGAGCCCCGCGGGGCCCAAGGTCCCCTGGTTCCCAAGAAAAGTGTCAGAGCTG
GACAAGTGTCATCACCTGGTCACCAAGTTCGACCCTGACCTGGACTTGGACCACCCGGGCTTCTCGGACCAGGTGTAC
CGCCAGCGCAGGAAGCTGATTGCTGAGATCGCCTTCCAGTACAGGCACGGCGACCCGATTCCCCGTGTGGAGTACACC
GCCGAGGAGATTGCCACCTGGAAGGAGGTCTACACCACGCTGAAGGGCCTCTACGCCACGCACGCCTGCGGGGAGCAC
CTGGAGGCCTTTGCTTTGCTGGAGCGCTTCAGCGGCTACCGGGAAGACAATATCCCCCAGCTGGAGGACGTCTCCCGC
TTCCTGAAGGAGCGCACGGGCTTCCAGCTGCGGCCTGTGGCCGGCCTGCTGTCCGCCCGGGACTTCCTGGCCAGCCTG
GCCTTCCGCGTGTTCCAGTGCACCCAGTATATCCGCCACGCGTCCTCGCCCATGCACTCCCCTGAGCCGGACTGCTGC
CACGAGCTGCTGGGGCACGTGCCCATGCTGGCCGACCGCACCTTCGCGCAGTTCTCGCAGGACATTGGCCTGGCGTCC
CTGGGGGCCTCGGATGAGGAAATTGAGAAGCTGTCCACGCTGTACTGGTTCACGGTGGAGTTCGGGCTGTGTAAGCAG
AACGGGGAGGTGAAGGCCTATGGTGCCGGGCTGCTGTCCTCCTACGGGGAGCTCCTGCACTGCCTGTCTGAGGAGCCT
GAGATTCGGGCCTTCGACCCTGAGGCTGCGGCCGTGCAGCCCTACCAAGACCAGACGTACCAGTCAGTCTACTTCGTG
TCTGAGAGCTTCAGTGACGCCAAGGACAAGCTCAGGAGCTATGCCTCACGCATCCAGCGCCCCTTCTCCGTGAAGTTC
GACCCGTACACGCTGGCCATCGACGTGCTGGACAGCCCCCAGGCCGTGCGGCGCTCCCTGGAGGGTGTCCAGGATGAG
CTGGACACCCTTGCCCATGCGCTGAGTGCCATTGGCTAAAATCAACCTCTGGATTACAAAATTTGTGAAAGATTGACT
GGTATTCTTAACTATGTTGCTCCTTTTACGCTATGTGGATACGCTGCTTTAATGCCTTTGTATCATGCTATTGCTTCC
CGTATGGCTTTCATTTTCTCCTCCTTGTATAAATCCTGGTTAGTTCTTGCCACGGCGGAACTCATCGCCGCCTGCCTT
GCCCGCTGCTGGACAGGGGCTCGGCTGTTGGGCACTGACAATTCCGTGGTGTTTATTTGTGAAATTTGTGATGCTATT
GCTTTATTTGTAACCATCTAGCTTTATTTGTGAAATTTGTGATGCTATTGCTTTATTTGTAACCATTATAAGCTGCAA
TAAACAAGTTAACAACAACAATTGCATTCATTTTATGTTTCAGGTTCAGGGGGAGAGCAATTGCATTCATTTTATGTT
TCAGGTTCAGGGGGAGGTGTGGGAGGTTTTTTAAAGCAAGTAAAACCTCTACAAATGTGGTAACTAGTCCACTCCCTC
TCTGCGCGCTCGCTCGCTCACTGAGGCCGGGCGACCAAAGGTCGCCCGACGCCCGGGCTTTGCCCGGGCGGCCTCAGT
GAGCGAGCGAGCGCGCAGAGAGGGACAGATCCGGGCCCGCATGCGTCGACAATTCACTGGCCGTCGTTTTACAACGTC
GTGACTGGGAAAACCCTGGCGTTACCCAACTTAATCGCCTTGCAGCACATCCCCCTTTCGCCAGCTGGCGTAATAGCG
AAGAGGCCCGCACCGATCGCCCTTCCCAACAGTTGCGCAGCCTGAATGGCGAATGGCGCCTGATGCGGTATTTTCTCC
TTACGCATCTGTGCGGTATTTCACACCGCATATGGTGCACTCTCAGTACAATCTGCTCTGATGCCGCATAGTTAAGCC
AGCCCCGACACCCGCCAACACCCGCTGACGCGCCCTGACGGGCTTGTCTGCTCCCGGCATCCGCTTACAGACAAGCTG
TGACCGTCTCCGGGAGCTGCATGTGTCAGAGGTTTTCACCGTCATCACCGAAACGCGCGAGACGAAAGGGCCTCGTGA
TACGCCTATTTTTATAGGTTAATGTCATGATAATAATGGTTTCTTAGACGTCAGGTGGCACTTTTCGGGGAAATGTGC
GCGGAACCCCTATTTGTTTATTTTTCTAAATACATTCAAATATGTATCCGCTCATGAGACAATAACCCTGATAAATGC
TTCAATAATATTGAAAAAGGAAGAGTATGAGTATTCAACATTTCCGTGTCGCCCTTATTCCCTTTTTTGCGGCATTTT
GCCTTCCTGTTTTTGCTCACCCAGAAACGCTGGTGAAAGTAAAAGATGCTGAAGATCAGTTGGGTGCACGAGTGGGTT
ACATCGAACTGGATCTCAACAGCGGTAAGATCCTTGAGAGTTTTCGCCCCGAAGAACGTTTTCCAATGATGAGCACTT
TTAAAGTTCTGCTATGTGGCGCGGTATTATCCCGTATTGACGCCGGGCAAGAGCAACTCGGTCGCCGCATACACTATT
CTCAGAATGACTTGGTTGAGTACTCACCAGTCACAGAAAAGCATCTTACGGATGGCATGACAGTAAGAGAATTATGCA
GTGCTGCCATAACCATGAGTGATAACACTGCGGCCAACTTACTTCTGACAACGATCGGAGGACCGAAGGAGCTAACCG
CTTTTTTGCACAACATGGGGGATCATGTAACTCGCCTTGATCGTTGGGAACCGGAGCTGAATGAAGCCATACCAAACG
ACGAGCGTGACACCACGATGCCTGTAGCAATGGCAACAACGTTGCGCAAACTATTAACTGGCGAACTACTTACTCTAG
CTTCCCGGCAACAATTAATAGACTGGATGGAGGCGGATAAAGTTGCAGGACCACTTCTGCGCTCGGCCCTTCCGGCTG
GCTGGTTTATTGCTGATAAATCTGGAGCCGGTGAGCGTGGGTCTCGCGGTATCATTGCAGCACTGGGGCCAGATGGTA
AGCCCTCCCGTATCGTAGTTATCTACACGACGGGGAGTCAGGCAACTATGGATGAACGAAATAGACAGATCGCTGAGA
TAGGTGCCTCACTGATTAAGCATTGGTAACTGTCAGACCAAGTTTACTCATATATACTTTAGATTGATTTAAAACTTC
ATTTTTAATTTAAAAGGATCTAGGTGAAGATCCTTTTTGATAATCTCATGACCAAAATCCCTTAACGTGAGTTTTCGT
TCCACTGAGCGTCAGACCCCGTAGAAAAGATCAAAGGATCTTCTTGAGATCCTTTTTTTCTGCGCGTAATCTGCTGCT
TGCAAACAAAAAAACCACCGCTACCAGCGGTGGTTTGTTTGCCGGATCAAGAGCTACCAACTCTTTTTCCGAAGGTAA
CTGGCTTCAGCAGAGCGCAGATACCAAATACTGTTCTTCTAGTGTAGCCGTAGTTAGGCCACCACTTCAAGAACTCTG
TAGCACCGCCTACATACCTCGCTCTGCTAATCCTGTTACCAGTGGCTGCTGCCAGTGGCGATAAGTCGTGTCTTACCG
GGTTGGACTCAAGACGATAGTTACCGGATAAGGCGCAGCGGTCGGGCTGAACGGGGGGTTCGTGCACACAGCCCAGCT
TGGAGCGAACGACCTACACCGAACTGAGATACCTACAGCGTGAGCTATGAGAAAGCGCCACGCTTCCCGAAGGGAGAA
AGGCGGACAGGTATCCGGTAAGCGGCAGGGTCGGAACAGGAGAGCGCACGAGGGAGCTTCCAGGGGGAAACGCCTGGT
ATCTTTATAGTCCTGTCGGGTTTCGCCACCTCTGACTTGAGCGTCGATTTTTGTGATGCTCGTCAGGGGGGCGGAGCC
TATGGAAAAACGCCAGCAACGCGGCCTTTTTACGGTTCCTGGCCTTTTGCTGGCCTTTTGCTCACATGTTCTTTCCTG
CGTTATCCCCTGATTCTGTGGATAACCGTATTACCGCCTTTGAGTGAGCTGATACCGCTCGCCGCAGCCGAACGACCG
AGCGCAGCGAGTCAGTGAGCGAGGAAGCGGAAGAGCGCCCAATACGCAAACCGCCTCTCCCCGCGCGTTGGCCGATTC
ATTAATGCAGCTGGCACGACAGGTTTCCCGACTGGAAAGCGGGCAGTGAGCGCAACGCAATTAATGTGAGTTAGCTCA
CTCATTAGGCACCCCAGGCTTTACACTTTATGCTTCCGGCTCGTATGTTGTGTGGAATTGTGAGCGGATAACAATTTC
ACACAGGAAACAGCTATGACCATGATTACGCCAAGCTCTCGAGATCTAGAAAGCTTCCCGGGGGGATCTG

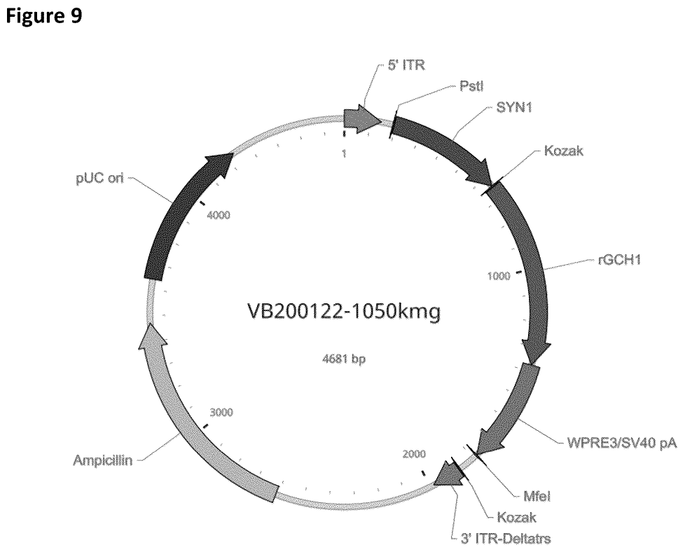

The following sequence, referred to herein as SEQ ID No: 20 and shown in FIG. 9, depicts a vector (i.e. the second expression vector of the composition of the first aspect) comprising a SYN1 promoter operably linked to a GCH-1 coding sequence:

[SEQ ID No: 20]
GGCCACTCCCTCTCTGCGCGCTCGCTCGCTCACTGAGGCCGGGCGACCAAAGGTCGCCCGACGCCCGGGCTTTGCCCG
GGCGGCCTCAGTGAGCGAGCGAGCGCGCAGAGAGGGAGTGGCCAACTCCATCACTAGGGGTTCCTGGAGGGGTGGAGT
CGTGACCTAGGCAACTTTGTATAGAAAAGTTGCTGCAGAGTGCAAGTGGGTTTTAGGACCAGGATGAGGCGGGGTGGG
GGTGCCTACCTGACGACCGACCCCGACCCACTGGACAAGCACCCAACCCCCATTCCCCAAATTGCGCATCCCCTATCA
GAGAGGGGGAGGGGAAACAGGATGCGGCGAGGCGCGTGCGCACTGCCAGCTTCAGCACCGCGGACAGTGCCTTCGCCC
CCGCCTGGCGGCGCGCGCCACCGCCGCCTCAGCACTGAAGGCGCGCTGACGTCACTCGCCGGTCCCCCGCAAACTCCC
CTTCCCGGCCACCTTGGTCGCGTCCGCGCCGCCGCCGGCCCAGCCGGACCGCACCACGCGAGGCGCGAGATAGGGGGG
CACGGGCGCGACCATCTGCGCTGCGGCGCCGGCGACTCAGCGCTGCCTCAGTCTGCGGTGGGCAGCGGAGGAGTCGTG
TCGTGCCTGAGAGCGCAGGCCACCATGGAGAAGCCGCGGGGTGTAAGGTGCACCAATGGGTTCCCCGAGCGGGAGCTG
CCGCGGCCCGGGGCCAGCCGACCTGCCGAGAAGTCCCGGCCGCCCGAGGCCAAGGGCGCACAGCCAGCCGACGCCTGG
AAGGCAGGGCGGCCCCGCAGCGAGGAGGATAACGAGCTGAACCTCCCCAACCTGGCGGCCGCTTACTCGTCCATCCTG
CGCTCGCTGGGCGAGGACCCCCAGCGGCAGGGGCTGCTCAAGACGCCCTGGAGGGCGGCCACCGCCATGCAGTTCTTC
ACCAAGGGATACCAGGAGACCATCTCAGATGTCCTGAACGATGCTATATTTGATGAGGACCATGACGAGATGGTGATT
GTGAAGGACATTGACATGTTTTCCATGTGTGAGCATCACCTGGTCCCATTTGTGGGAAGGGTCCATATTGGTTATCTT
CCTAACAAGCAAGTCCTTGGTCTCAGCAAACTTGCCAGGATTGTGGAAATCTACAGTAGAAGACTACAAGTTCAAGAA
CGCCTTACCAAACAGATTGCAGTGGCCATCACAGAAGCCTTGCAGCCTGCTGGCGTCGGGGTAGTGATTGAAGCAACA
CACATGTGTATGGTCATGCGAGGTGTGCAGAAAATGAACAGCAAGACTGTCACTAGCACCATGCTAGGCGTGTTCCGG
GAAGACCCAAAGACTCGGGAGGAGTTCCTCACACTCATCAGGAGCTGAAATCAACCTCTGGATTACAAAATTTGTGAA
AGATTGACTGGTATTCTTAACTATGTTGCTCCTTTTACGCTATGTGGATACGCTGCTTTAATGCCTTTGTATCATGCT
ATTGCTTCCCGTATGGCTTTCATTTTCTCCTCCTTGTATAAATCCTGGTTAGTTCTTGCCACGGCGGAACTCATCGCC
GCCTGCCTTGCCCGCTGCTGGACAGGGGCTCGGCTGTTGGGCACTGACAATTCCGTGGTGTTTATTTGTGAAATTTGT
GATGCTATTGCTTTATTTGTAACCATCTAGCTTTATTTGTGAAATTTGTGATGCTATTGCTTTATTTGTAACCATTAT
AAGCTGCAATAAACAAGTTAACAACAACAATTGCATTCATTTTATGTTTCAGGTTCAGGGGGAGAGCAATTGCATTCA
TTTTATGTTTCAGGTTCAGGGGGAGGTGTGGGAGGTTTTTTAAAGCAAGTAAAACCTCTACAAATGTGGTAACTAGTC
CACTCCCTCTCTGCGCGCTCGCTCGCTCACTGAGGCCGGGCGACCAAAGGTCGCCCGACGCCCGGGCTTTGCCCGGGC
GGCCTCAGTGAGCGAGCGAGCGCGCAGAGAGGGACAGATCCGGGCCCGCATGCGTCGACAATTCACTGGCCGTCGTTT
TACAACGTCGTGACTGGGAAAACCCTGGCGTTACCCAACTTAATCGCCTTGCAGCACATCCCCCTTTCGCCAGCTGGC
GTAATAGCGAAGAGGCCCGCACCGATCGCCCTTCCCAACAGTTGCGCAGCCTGAATGGCGAATGGCGCCTGATGCGGT
ATTTTCTCCTTACGCATCTGTGCGGTATTTCACACCGCATATGGTGCACTCTCAGTACAATCTGCTCTGATGCCGCAT
AGTTAAGCCAGCCCCGACACCCGCCAACACCCGCTGACGCGCCCTGACGGGCTTGTCTGCTCCCGGCATCCGCTTACA
GACAAGCTGTGACCGTCTCCGGGAGCTGCATGTGTCAGAGGTTTTCACCGTCATCACCGAAACGCGCGAGACGAAAGG
GCCTCGTGATACGCCTATTTTTATAGGTTAATGTCATGATAATAATGGTTTCTTAGACGTCAGGTGGCACTTTTCGGG
GAAATGTGCGCGGAACCCCTATTTGTTTATTTTTCTAAATACATTCAAATATGTATCCGCTCATGAGACAATAACCCT
GATAAATGCTTCAATAATATTGAAAAAGGAAGAGTATGAGTATTCAACATTTCCGTGTCGCCCTTATTCCCTTTTTTG
CGGCATTTTGCCTTCCTGTTTTTGCTCACCCAGAAACGCTGGTGAAAGTAAAAGATGCTGAAGATCAGTTGGGTGCAC
GAGTGGGTTACATCGAACTGGATCTCAACAGCGGTAAGATCCTTGAGAGTTTTCGCCCCGAAGAACGTTTTCCAATGA
TGAGCACTTTTAAAGTTCTGCTATGTGGCGCGGTATTATCCCGTATTGACGCCGGGCAAGAGCAACTCGGTCGCCGCA
TACACTATTCTCAGAATGACTTGGTTGAGTACTCACCAGTCACAGAAAAGCATCTTACGGATGGCATGACAGTAAGAG
AATTATGCAGTGCTGCCATAACCATGAGTGATAACACTGCGGCCAACTTACTTCTGACAACGATCGGAGGACCGAAGG
AGCTAACCGCTTTTTTGCACAACATGGGGGATCATGTAACTCGCCTTGATCGTTGGGAACCGGAGCTGAATGAAGCCA
TACCAAACGACGAGCGTGACACCACGATGCCTGTAGCAATGGCAACAACGTTGCGCAAACTATTAACTGGCGAACTAC
TTACTCTAGCTTCCCGGCAACAATTAATAGACTGGATGGAGGCGGATAAAGTTGCAGGACCACTTCTGCGCTCGGCCC
TTCCGGCTGGCTGGTTTATTGCTGATAAATCTGGAGCCGGTGAGCGTGGGTCTCGCGGTATCATTGCAGCACTGGGGC
CAGATGGTAAGCCCTCCCGTATCGTAGTTATCTACACGACGGGGAGTCAGGCAACTATGGATGAACGAAATAGACAGA
TCGCTGAGATAGGTGCCTCACTGATTAAGCATTGGTAACTGTCAGACCAAGTTTACTCATATATACTTTAGATTGATT
TAAAACTTCATTTTTAATTTAAAAGGATCTAGGTGAAGATCCTTTTTGATAATCTCATGACCAAAATCCCTTAACGTG
AGTTTTCGTTCCACTGAGCGTCAGACCCCGTAGAAAAGATCAAAGGATCTTCTTGAGATCCTTTTTTTCTGCGCGTAA
TCTGCTGCTTGCAAACAAAAAAACCACCGCTACCAGCGGTGGTTTGTTTGCCGGATCAAGAGCTACCAACTCTTTTTC
CGAAGGTAACTGGCTTCAGCAGAGCGCAGATACCAAATACTGTTCTTCTAGTGTAGCCGTAGTTAGGCCACCACTTCA
AGAACTCTGTAGCACCGCCTACATACCTCGCTCTGCTAATCCTGTTACCAGTGGCTGCTGCCAGTGGCGATAAGTCGT
GTCTTACCGGGTTGGACTCAAGACGATAGTTACCGGATAAGGCGCAGCGGTCGGGCTGAACGGGGGGTTCGTGCACAC
AGCCCAGCTTGGAGCGAACGACCTACACCGAACTGAGATACCTACAGCGTGAGCTATGAGAAAGCGCCACGCTTCCCG
AAGGGAGAAAGGCGGACAGGTATCCGGTAAGCGGCAGGGTCGGAACAGGAGAGCGCACGAGGGAGCTTCCAGGGGGAA
ACGCCTGGTATCTTTATAGTCCTGTCGGGTTTCGCCACCTCTGACTTGAGCGTCGATTTTTGTGATGCTCGTCAGGGG
GGCGGAGCCTATGGAAAAACGCCAGCAACGCGGCCTTTTTACGGTTCCTGGCCTTTTGCTGGCCTTTTGCTCACATGT
TCTTTCCTGCGTTATCCCCTGATTCTGTGGATAACCGTATTACCGCCTTTGAGTGAGCTGATACCGCTCGCCGCAGCC
GAACGACCGAGCGCAGCGAGTCAGTGAGCGAGGAAGCGGAAGAGCGCCCAATACGCAAACCGCCTCTCCCCGCGCGTT
GGCCGATTCATTAATGCAGCTGGCACGACAGGTTTCCCGACTGGAAAGCGGGCAGTGAGCGCAACGCAATTAATGTGA
GTTAGCTCACTCATTAGGCACCCCAGGCTTTACACTTTATGCTTCCGGCTCGTATGTTGTGTGGAATTGTGAGCGGAT
AACAATTTCACACAGGAAACAGCTATGACCATGATTACGCCAAGCTCTCGAGATCTAGAAAGCTTCCCGGGGGGATCT
G

The invention preferably provides a composition comprising:—
(i) a first self-complementary adeno-associated virus (scAAV) vector comprising a promoter operably linked to a coding sequence, which encodes tyrosine hydroxylase (TH), optionally human truncated TH lacking the regulatory domain; and
(ii) a second self-complementary adeno-associated virus (scAAV) vector comprising a promoter operably linked a coding sequence, which encodes GTP cyclohydrolase 1 (GCH1).

According to a second aspect, there is provided a pharmaceutical composition comprising the composition according to the first aspect, and a pharmaceutically acceptable vehicle.

The composition comprising the recombinant vectors of the first aspect, and the pharmaceutical composition of the second aspect are particularly suitable for therapy.

Hence, according to a third aspect, there is provided the composition according to the first aspect, or the pharmaceutical composition according to the second aspect, for use as a medicament or in therapy.

Treatment of Parkinson's disease, DOPA responsive dystonia, vascular parkinsonism, side effects associated with L-DOPA treatment for Parkinson's disease, L-DOPA induced dyskinesia, Segawa syndrome, or genetic dopamine receptor abnormalities are especially envisaged.

Thus, in a fourth aspect of the invention, there is provided the composition according to the first aspect, or the pharmaceutical composition according to the second aspect, for use in treating, preventing, or ameliorating Parkinson's disease, DOPA responsive dystonia, vascular parkinsonism, side effects associated with L-DOPA treatment for Parkinson's disease, L-DOPA induced dyskinesia, Segawa syndrome, or genetic dopamine receptor abnormalities.

The skilled person will appreciate that the therapeutic use of the first and second vectors means that they do not necessarily need to be provided in the same composition or formulation.

Hence, in yet another aspect, there is provided a first expression vector comprising a promoter operably linked to a self-complementary coding sequence, which encodes tyrosine hydroxylase (TH), and a second expression vector comprising a promoter operably linked to a coding sequence, which encodes GTP cyclohydrolase 1 (GCH1), for use in therapy.

In yet another aspect, there is provided a first expression vector comprising a promoter operably linked to a self-complementary coding sequence, which encodes tyrosine hydroxylase (TH), and a second expression vector comprising a promoter operably linked to a coding sequence, which encodes GTP cyclohydrolase 1 (GCH1), for use in treating, preventing, or ameliorating Parkinson's disease, DOPA responsive dystonia, vascular parkinsonism, side effects associated with L-DOPA treatment for Parkinson's disease, L-DOPA induced dyskinesia, Segawa syndrome, or genetic dopamine receptor abnormalities.

The first and second expression vectors used herein are as described in relation to the first aspect.

Preferably, the uses described herein comprise the use of a first self-complementary adeno-associated virus (scAAV) vector comprising a promoter operably linked to a coding sequence, which encodes tyrosine hydroxylase (TH), optionally human truncated TH lacking the regulatory domain.

Preferably, the uses described herein comprise the use of a second self-complementary adeno-associated virus (scAAV) vector comprising a promoter operably linked a coding sequence, which encodes GTP cyclohydrolase 1 (GCH1).

According to a fifth aspect, there is provided a method of treating, preventing, or ameliorating Parkinson's disease, DOPA responsive dystonia, vascular parkinsonism, side effects associated with L-DOPA treatment for Parkinson's disease, L-DOPA induced dyskinesia, Segawa syndrome, or genetic dopamine receptor abnormalities in a subject, the method comprising administering, to a subject in need of such treatment, a therapeutically effective amount of the composition according to the first aspect, or the composition according to the second aspect.

In yet another aspect, there is provided a method of treating, preventing, or ameliorating Parkinson's disease, DOPA responsive dystonia, vascular parkinsonism, side effects associated with L-DOPA treatment for Parkinson's disease, L-DOPA induced dyskinesia, Segawa syndrome, or genetic dopamine receptor abnormalities in a subject, the method comprising administering, to a subject in need of such treatment, a therapeutically effective amount of a first expression vector comprising a promoter operably linked to a self-complementary coding sequence, which encodes tyrosine hydroxylase (TH), and a second expression vector comprising a promoter operably linked to a coding sequence, which encodes GTP cyclohydrolase 1 (GCH1).

Preferably, the first and second vectors or compositions according to the invention are used in a gene therapy technique.

In embodiment, the disorder to be treated is selected from the group consisting of Parkinson's disease, DOPA responsive dystonia, vascular parkinsonism, side effects associated with L-DOPA treatment for Parkinson's disease, L-DOPA induced dyskinesia, Segawa syndrome, or genetic dopamine receptor abnormalities.

In a most preferred embodiment, the disease to be treated is Parkinson's disease.

The disclosed gene therapy technique leads to a constant level of production of L-DOPA in the striatum. This removes or reduces the need for oral L-DOPA and so results in reduced peak to trough variation. Hence, the disclosed gene therapy can be used for the treatment of side effects associated with L-DOPA treatment of Parkinson's disease and of L-DOPA-induced dyskinesia.

The disclosed gene therapy technique may be used for the treatment of Segawa syndrome. Although it would be possible to treat Segawa syndrome with a gene therapy delivering only GCH1, or only TH (depending on the cause of the Segawa syndrome) the additional inclusion of TH or GCH1 (respectively) is not expected to be prejudicial and may be beneficial. The disclosed treatment is especially advantageous as, due to the rareness of Segawa syndrome, it may not be commercially attractive or viable to develop a treatment solely for this indication. Production of the disclosed invention for this indication as well as Parkinson's disease, will reduce the unit cost of the therapy.

In a preferred embodiment, medicaments according to the invention (i.e. the first and second expression vectors) may be administered to a subject by injection into the blood stream, the cerebrospinal fluid, a nerve, or directly into a site requiring treatment. For instance, the expression vectors may be delivered to the brain. The vector may be delivered bilaterally or unilaterally. Specific regions of the brain may be targeted, such as striatum. The putamen or caudate nucleus may be targeted. Alternatively, only the putamen may be targeted. The treatment may be centred on the dopaminergic neurons of the pars compacta region in the substantia nigra.

The delivery method may be direct injection. Methods for injection into the brain (for instance the striatum) are well known in the art (Bilang-Bleuel et al (1997) Proc. Acad. Nati. Sci. USA 94:8818-8823; Choi-Lundberg et al (1998) Exp. Neurol. 154:261-275; Choi-Lundberg et al (1997) Science 275:838-841; and Mandel et al (1997)) Proc. Acad. Natl. Sci. USA 94:14083-14088). Alternatively, or in addition, the vector chosen may have a tropism that is targeted towards a specific desired tissue, such as a neuron.

Modifications of the vector capsid properties could enable targeting of the vector to the striatal region also after intrathecal (IT) injection or injection into the cerebral ventricles (ICV). Injections into the ventricles, such that the resulting L-DOPA or dopamine is elevated, may be transmitted to the striatum by diffusion through CSF or axonal transfer. An alternative approach is to generate chimeric AAV serotypes that would inherit different binding properties from the two serotypes mixed.

Preferably, however, the vector and compositions according to the invention may be administered to a subject by injection into the striatum.

The gene therapy vectors may be produced by any technique known in the art. For instance, the AAV vectors may be produced using classic triple transfection methodology. Methods for the production of adeno-associated virus vectors are disclosed in Matsushita et al. (Matsushita et al., Adeno-associated virus vectors can be efficiently produced without helper virus. Gene Therapy (1998) 5, 938-945).

It will be appreciated that the amount of the composition of the invention and amount of each of the two vectors within the mixture or provided separately that is required is determined by the biological activity and bioavailability of the two vectors in the mixture (or separately) which in turn depends on the mode of administration, the physiochemical properties of the vectors and whether the composition is being used as a monotherapy or combined with other therapies. Optimal dosages to be administered may be determined by those skilled in the art, and will vary with the particular expression vectors in use, the strength of the composition and pharmaceutical composition, the mode of administration, and the advancement of the neurodegenerative disorder being treated. Additional factors depending on the particular subject being treated will result in a need to adjust dosages, including subject age, weight, gender, diet, and time of administration.

The dose of composition of the invention delivered may be 300 μl to 20,000 μl, 300 μl to 10,000 μl, 300 μl to 5,000 μl, 300 μl to 4500 μl, 400 μl to 4000 μl, 500 μl to 3500 μl, 600 μl to 3000 μl, 700 μl to 2500 μl, 750 μl to 2000 μl, 800 μl to 1500 μl, 850 μl to 1000 μl, or approximately 900 μl.

If administered as a mixture of AAV vectors, the titre of each AAV may be 1E8 to 5E14, 1E9 to 1E14, 1E10 to 5E13, 1E11 to 1E13, 1E12 to 8E12, 4E12 to 6E12, or roughly 5E12 genome copies per ml (GC/ml).

If administered as a mixture of naked DNA plasmid vectors, the dose of each DNA plasmid vector may be 50, 100, 200, 300, 400, 500, 600, 700, 800, 900, 1000, 1250, 1500, 1750 or 2000 micrograms (μg) per brain hemisphere.

The composition may be administered during or after onset of the disorder. Doses may be given as a single administration, or multiple doses may be given over the course of the treatment. A dose may be administered to a patient, and the patient may be monitored in order to assess the necessity for a second or further doses. Repeat use delivery of the same genomes within AAV vectors may be facilitated by the switching the AAV capsid serotype to reduce the probability of interference by an antibody or cell mediated immune response induced by the previous treatment.

In some embodiments, the therapeutic methods may include, prior to gene therapy treatment, a test infusion of L-DOPA. The test infusion may be used to demonstrate that a subject is responsive to L-DOPA and benefits from reduced peak to trough variation in plasma and or brain L-DOPA levels, and so may allow the selection of subjects most likely to benefit from gene therapy treatment. The L-DOPA test infusion may be by any means capable of creating a steady blood level over hours or days. Examples of suitable infusion methods include by nasogastric tube, i.v. infusion, infusion via a pump, by the use of DuoDOPA, or any other suitable means.

It will be appreciated that the first and second expression vectors on their own, or the composition according to the first aspect, or the pharmaceutical composition of the second aspect may be used in a medicament, which may be used as a monotherapy (i.e. use of vector composition according to the first aspect or the composition according to the second aspect of the invention), for treating, ameliorating, or preventing any disorder as disclosed herein. Alternatively, the vectors on their own or the composition according to the invention may be used as an adjunct to, or in combination with, known therapies for treating, ameliorating, or preventing any disorder as disclosed herein. In some cases, vectors may be used as an adjunct to, in combination with, or alongside a treatment designed to improve the gene therapy. For instance, the vectors may be used in combination with an immunosuppressive treatment, in order to reduce, prevent, or control an immune response induced by the gene therapy itself. For example, the immunosuppressive treatment may prevent, reduce, or control an immune response directed to a capsid of a gene therapy vector, a genome comprised within a gene therapy vector, or a product produced by a gene therapy vector during therapy. The immunosuppressive regime may include a general immunosuppressant, such as a steroid. The immunosuppressive regime may include more targeted immunosuppression designed to reduce specific immune responses, such as immunotherapy to specific antigens found within, or produced by, a gene therapy construct. Alternatively, the composition comprising the vectors may be administered or used in combination with an agent intended to increase the efficiency of uptake of the vectors by the target cells, or increase the efficiency of transfection or transduction or prevent down-regulation or silencing of expression.

The compositions according to the invention may be combined in compositions having a number of different forms depending, in particular, on the manner in which the composition is to be used. Thus, for example, the compositions may be in the form of a powder, liquid, micellar solution, liposome suspension or any other suitable form that may be administered to a person or animal in need of treatment. It will be appreciated that the vehicle of medicaments according to the invention should be one which is well-tolerated by the subject to whom it is given. Preferably, the composition is in the form of an injectable liquid.

Known procedures, such as those conventionally employed by the pharmaceutical industry (e.g. in vivo experimentation, clinical trials, etc.), may be used to form specific formulations of the composition according to the invention and precise therapeutic regimes.

According to a sixth aspect, there is provided a method of preparing the pharmaceutical composition according to the second aspect, the method comprising contacting the composition according to the first aspect, and a pharmaceutically acceptable vehicle.

A “subject” may be a vertebrate, mammal, or domestic animal. Hence, compositions and medicaments according to the invention may be used to treat any mammal, for example livestock (e.g. a horse), pets, or may be used in other veterinary applications. Most preferably, however, the subject is a human being.

A “therapeutically effective amount” of the vector or the composition is any amount which, when administered to a subject, is the amount of the aforementioned that is needed to treat the disorder.

A “pharmaceutically acceptable vehicle” as referred to herein, is any known compound or combination of known compounds that are known to those skilled in the art to be useful in formulating pharmaceutical compositions.

In a preferred embodiment, the pharmaceutically acceptable vehicle may be such as to allow injection of the composition directly into a subject. For instance, the vehicle may be suitable for allowing the injection of the composition into the striatum.

In one embodiment, the pharmaceutically acceptable vehicle may be a solid, and the composition may be in the form of a powder, or suspension. A solid pharmaceutically acceptable vehicle may include one or more substances which may also act as, lubricants, solubilisers, suspending agents, dyes, fillers, glidants, compression aids, inert binders, preservatives, dyes, coatings, or solid-disintegrating agents. The vehicle may also be an encapsulating material. In powders, the vehicle is a finely divided solid that is in admixture with the finely divided active agents according to the invention. In another embodiment, the pharmaceutical vehicle may be a gel or the like.

However, the pharmaceutical vehicle may be a suspension or a liquid, and the pharmaceutical composition is in the form of a suspension or a solution. Liquid pharmaceutical compositions, which are sterile solutions or suspensions, can be utilized by, for example, intrathecal, epidural, intravenous and particularly direct injection into the target area of brain, such as the striatum. The first and second vectors may be prepared as a suspension or as sterile solid or dry composition that may be dissolved or suspended at the time of administration using sterile water, saline, Dulbecco's Phosphate Buffered Saline (dPBS) with MgCl2 and CaCl2), artificial cerebrospinal fluid or other appropriate sterile injectable medium.

In one embodiment, the composition of the first and second aspects of the invention may be supplied as a single pre-mixed formulation (e.g. in a vial or syringe). In another embodiment, however, the composition comprises the two expression vectors supplied individually (e.g. in two vials or two syringes), but in a kit, and mixed immediately prior to, or at the time of, administration.

Thus, in a seventh aspect, there is provided a kit of parts comprising the first and second expression vectors as defined in accordance with the first aspect, and optionally, instructions for use.

The kit of parts may comprise a first container in which the first expression vector is contained. The kit of parts may comprise a second container in which the second expression vector is contained. The first and/or second container may be a vial, syringe, Eppendorf, or the like. For example, the syringe may be a pre-loaded syringe. The kit may comprise a mixing vessel in which the vectors may be mixed prior to administration. Alternatively, one vector may be transferred to the container holding the other vector, where they may be mixed. Alternatively, the vectors may be administered separately, but sufficiently contemporaneously such that they are simultaneously therapeutically active in the subject. The instructions for use preferably describe how to mix the vectors, if appropriate, and dosages.

The ratio of the first expression vector to second expression vector may preferably be about 50:50, but could be 5:95, 10:90, 20:80, 30:70 60:40, 40:60, 70:30, 80:20, 90:10 or 95:5.

It will be appreciated that the kit of the seventh aspect can be used in therapy, and preferably for treating, preventing, or ameliorating Parkinson's disease, DOPA responsive dystonia, vascular parkinsonism, side effects associated with L-DOPA treatment for Parkinson's disease, L-DOPA induced dyskinesia, Segawa syndrome, or genetic dopamine receptor abnormalities.

In another aspect, there is provided a composition comprising first and second expression vectors, wherein the first expression vector comprises a promoter operably linked to a self-complementary coding sequence, which encodes tyrosine hydroxylase (TH), and the second expression vector comprises a coding sequence, which encodes GTP cyclohydrolase 1 (GCH1).

It will be appreciated that the invention extends to any nucleic acid or peptide or variant, derivative or analogue thereof, which comprises substantially the amino acid or nucleic acid sequences of any of the sequences referred to herein, including variants or fragments thereof. The terms “substantially the amino acid/nucleotide/peptide sequence”, “variant” and “fragment”, can be a sequence that has at least 40% sequence identity with the amino acid/nucleotide/peptide sequences of any one of the sequences referred to herein, for example 40% identity with the sequence identified as SEQ ID No:1-29 and so on.

Amino acid/polynucleotide/polypeptide sequences with a sequence identity which is greater than 65%, more preferably greater than 70%, even more preferably greater than 75%, and still more preferably greater than 80% sequence identity to any of the sequences referred to are also envisaged. Preferably, the amino acid/polynucleotide/polypeptide sequence has at least 85% identity with any of the sequences referred to, more preferably at least 90% identity, even more preferably at least 92% identity, even more preferably at least 95% identity, even more preferably at least 97% identity, even more preferably at least 98% identity and, most preferably at least 99% identity with any of the sequences referred to herein.

The skilled technician will appreciate how to calculate the percentage identity between two amino acid/polynucleotide/polypeptide sequences. In order to calculate the percentage identity between two amino acid/polynucleotide/polypeptide sequences, an alignment of the two sequences must first be prepared, followed by calculation of the sequence identity value. The percentage identity for two sequences may take different values depending on:—(i) the method used to align the sequences, for example, ClustalW, BLAST, FASTA, Smith-Warterman (implemented in different programs), or structural alignment from 3D comparison; and (ii) the parameters used by the alignment method, for example, local vs global alignment, the pair-score matrix used (e.g. BLOSUM62, PAM250, Gonnet etc.), and gap-penalty, e.g. functional form and constants.

Having made the alignment, there are many different ways of calculating percentage identity between the two sequences. For example, one may divide the number of identities by: (i) the length of shortest sequence; (ii) the length of alignment; (iii) the mean length of sequence; (iv) the number of non-gap positions; or (v) the number of equivalenced positions excluding overhangs. Furthermore, it will be appreciated that percentage identity is also strongly length dependent. Therefore, the shorter a pair of sequences is, the higher the sequence identity one may expect to occur by chance.

Hence, it will be appreciated that the accurate alignment of protein or DNA sequences is a complex process. The popular multiple alignment program ClustalW (Thompson et al., 1994, Nucleic Acids Research, 22, 4673-4680; Thompson et al., 1997, Nucleic Acids Research, 24, 4876-4882) is a preferred way for generating multiple alignments of proteins or DNA in accordance with the invention. Suitable parameters for ClustalW may be as follows: For DNA alignments: Gap Open Penalty=15.0, Gap Extension Penalty=6.66, and Matrix=Identity. For protein alignments: Gap Open Penalty=10.0, Gap Extension Penalty=0.2, and Matrix=Gonnet. For DNA and Protein alignments: ENDGAP=−1, and GAPDIST=4. Those skilled in the art will be aware that it may be necessary to vary these and other parameters for optimal sequence alignment.

Preferably, calculation of percentage identities between two amino acid/polynucleotide/polypeptide sequences may then be calculated from such an alignment as (N/T)*100, where N is the number of positions at which the sequences share an identical residue, and T is the total number of positions compared including gaps and either including or excluding overhangs. Preferably, overhangs are included in the calculation. Hence, a most preferred method for calculating percentage identity between two sequences comprises (i) preparing a sequence alignment using the ClustalW program using a suitable set of parameters, for example, as set out above; and (ii) inserting the values of N and T into the following formula:—Sequence Identity=(N/T)*100.

Alternative methods for identifying similar sequences will be known to those skilled in the art. For example, a substantially similar nucleotide sequence will be encoded by a sequence which hybridizes to DNA sequences or their complements under stringent conditions. By stringent conditions, we mean the nucleotide hybridises to filter-bound DNA or RNA in 3× sodium chloride/sodium citrate (SSC) at approximately 45° C. followed by at least one wash in 0.2×SSC/0.1% SDS at approximately 20-65° C. Alternatively, a substantially similar polypeptide may differ by at least 1, but less than 5, 10, 20, 50 or 100 amino acids from the sequences shown in, for example, in the amino acid sequence that are included within SEQ ID Nos: 1-29.

Due to the degeneracy of the genetic code, it is clear that any nucleic acid sequence described herein could be varied or changed without substantially affecting the sequence of the protein encoded thereby, to provide a functional variant thereof. Suitable nucleotide variants are those having a sequence altered by the substitution of different codons that encode the same amino acid within the sequence, thus producing a silent change. Other suitable variants are those having homologous nucleotide sequences but comprising all, or portions of, sequence, which are altered by the substitution of different codons that encode an amino acid with a side chain of similar biophysical properties to the amino acid it substitutes, to produce a conservative change. For example, small non-polar, hydrophobic amino acids include glycine, alanine, leucine, isoleucine, valine, proline, and methionine. Large non-polar, hydrophobic amino acids include phenylalanine, tryptophan and tyrosine. The polar neutral amino acids include serine, threonine, cysteine, asparagine and glutamine. The positively charged (basic) amino acids include lysine, arginine and histidine. The negatively charged (acidic) amino acids include aspartic acid and glutamic acid. It will therefore be appreciated which amino acids may be replaced with an amino acid having similar biophysical properties, and the skilled technician will know the nucleotide sequences encoding these amino acids.

All of the features described herein (including any accompanying claims, abstract and drawings), and/or all of the steps of any method or process so disclosed, may be combined with any of the above aspects in any combination, except combinations where at least some of such features and/or steps are mutually exclusive.

For a better understanding of the invention, and to show how embodiments of the same may be carried into effect, reference will now be made, by way of example, to the accompanying Figures, in which:—

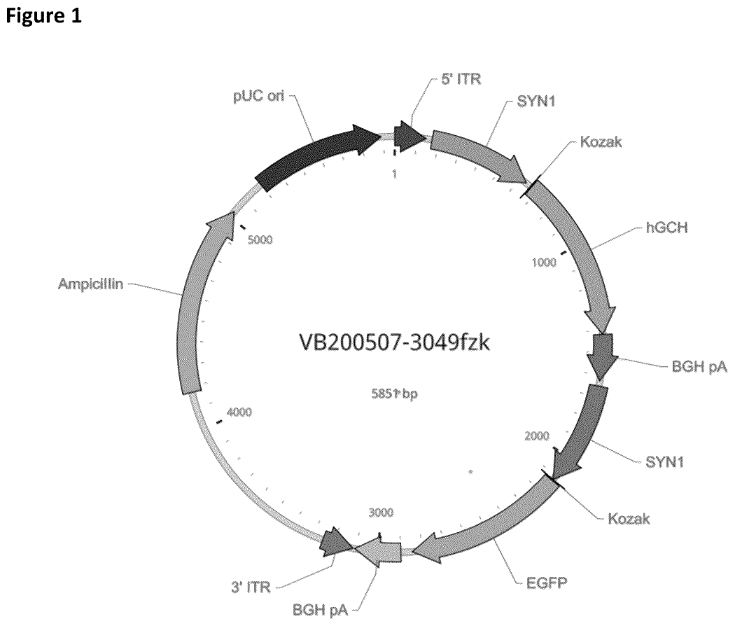

FIG. 1 is a plasmid map of one embodiment of a single-stranded ssAAV-SYN1-hGCH-SYN1-EGFP-WPRE-pA vector (construct A).

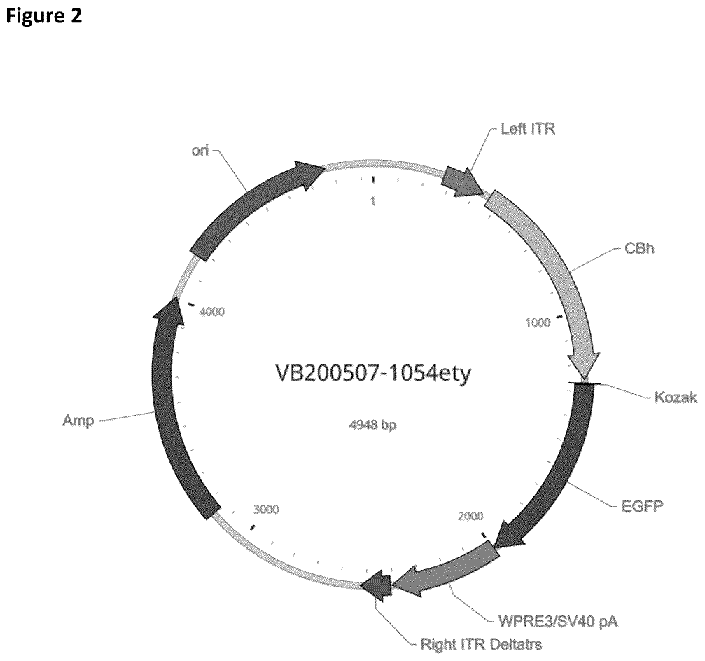

FIG. 2 is a plasmid map of one embodiment of a self-complementary plasmid pscAAV-CBh-EGFP-WPRE-SV40pA vector (construct B).

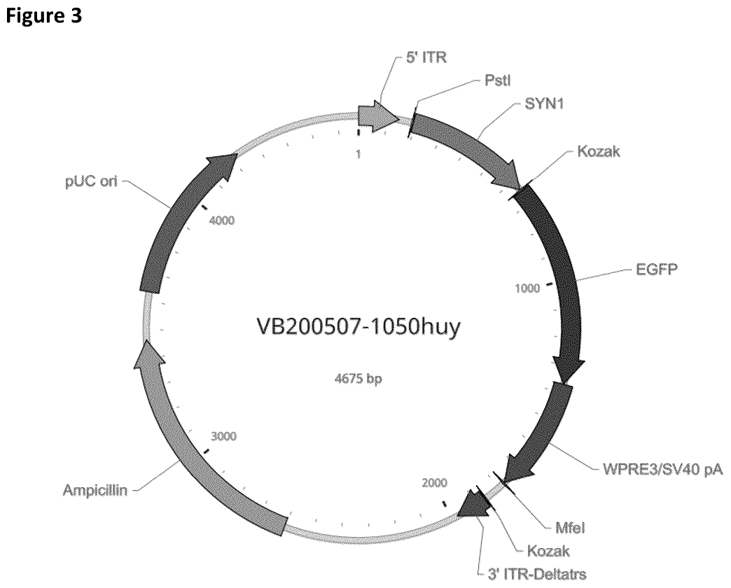

FIG. 3 is a plasmid map of one embodiment of a self-complementary plasmid pscAAV-SYN1-EGFP-WPRE-SV40pA vector (construct C).

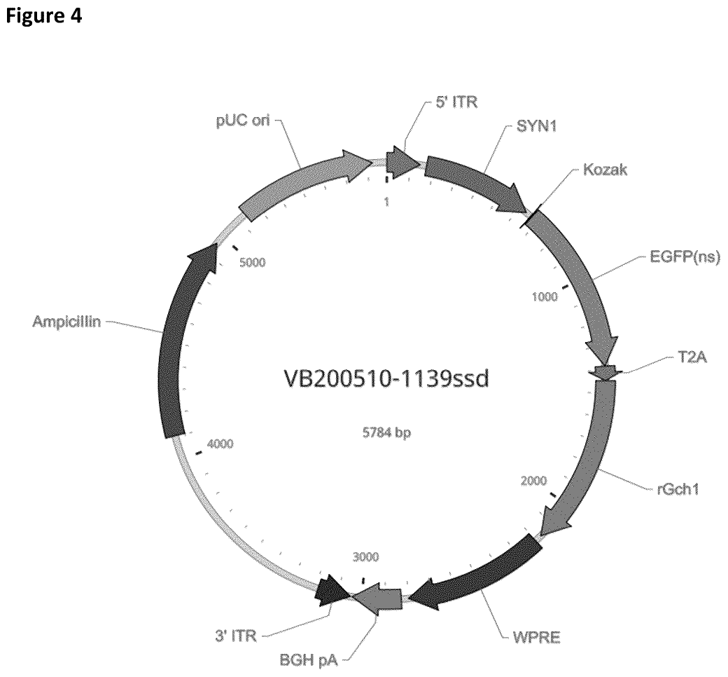

FIG. 4 is a plasmid map of one embodiment of a single-stranded plasmid sspAAV-SYN1-EGFP-T2A-GCH-WPRE-pA vector (construct D).

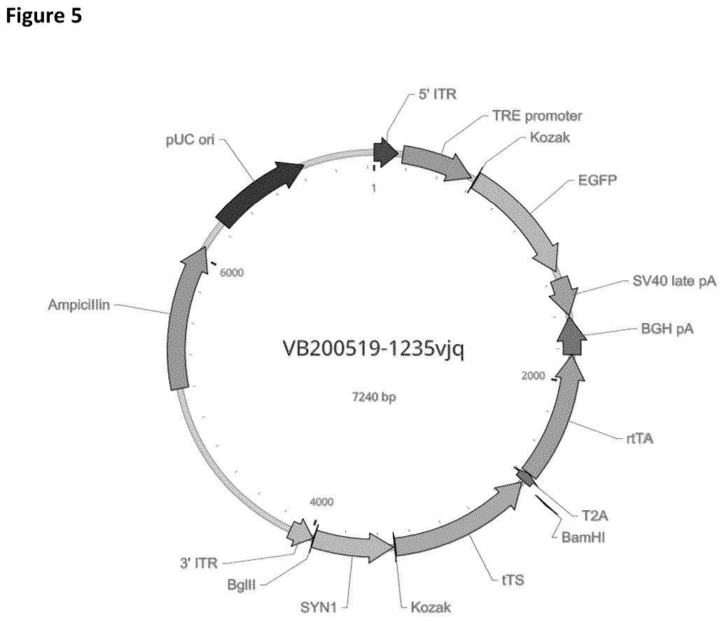

FIG. 5 is a plasmid map of one embodiment of an pAAV[TetOn]TRE-EGFP-rev(SYN1-tTS-T2A-rtTA) vector (construct E).

FIG. 6 is a plasmid map of one embodiment of a vector comprising a CBh promoter operably linked to a htTH coding sequence.

FIG. 7 is a plasmid map of one embodiment of a vector comprising a CBh promoter operably linked to a GCH-1 coding sequence.

FIG. 8 is a plasmid map of one embodiment of a vector comprising a SYN1 promoter operably linked to a htTH coding sequence.

FIG. 9 is a plasmid map of one embodiment of a vector comprising a SYN1 promoter operably linked to a GCH-1 coding sequence.

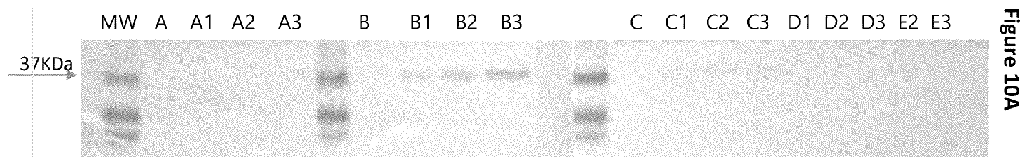

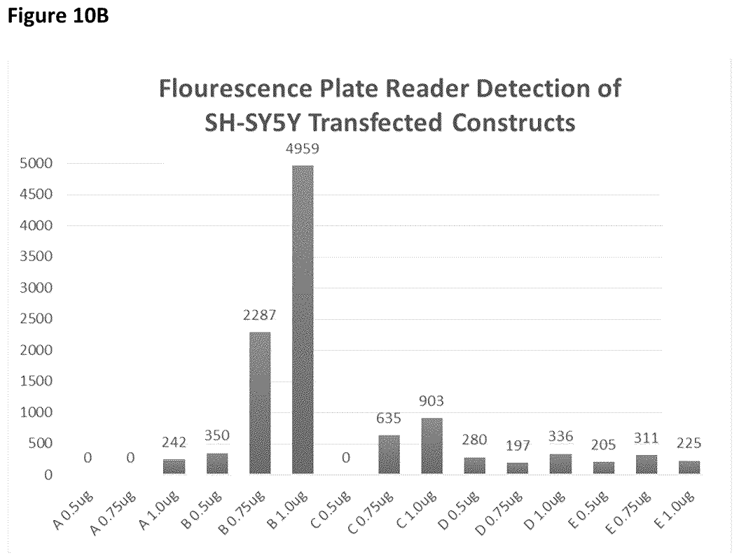

FIG. 10A shows qualitative analysis by Western blot of expression of the reporter gene EGFP of a number of constructs, A, B and C. The blot shows the correct molecular weight of EGFP of 37 KDa; and FIG. 10B shows quantitative DAB colorimetric detection of expression of the reporter gene EGFP.

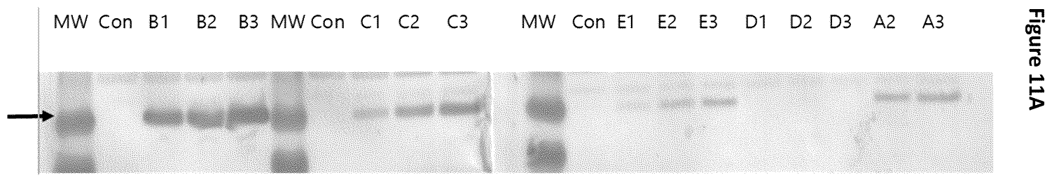

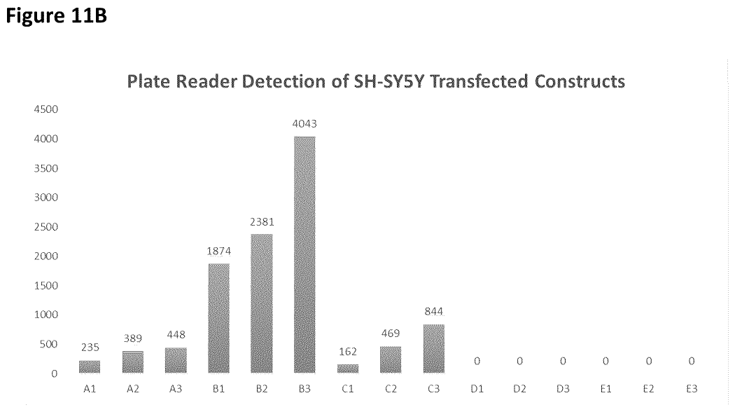

FIG. 11A shows a second data set of qualitative analysis by Western blot of expression of the reporter gene EGFP of constructs, A, B and C. The blot shows the correct molecular weight of EGFP of 37 KDa, and FIG. 11B shows quantitative DAB colorimetric detection of expression of the reporter gene EGFP. The results confirm that the B construct shows the highest dose dependent expression of EGFP.

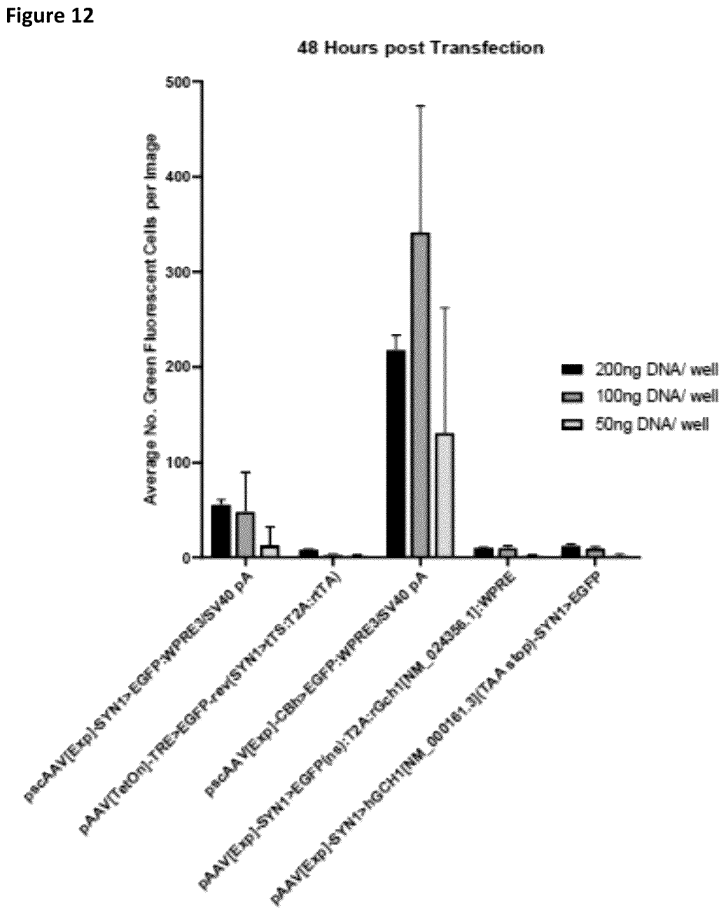

FIG. 12 shows the expression levels of GFP for the tested constructs 48 hours post transfection.

FIG. 13 shows the average number of GFP cells plotted over time with a maximum signal at about 48 hours.

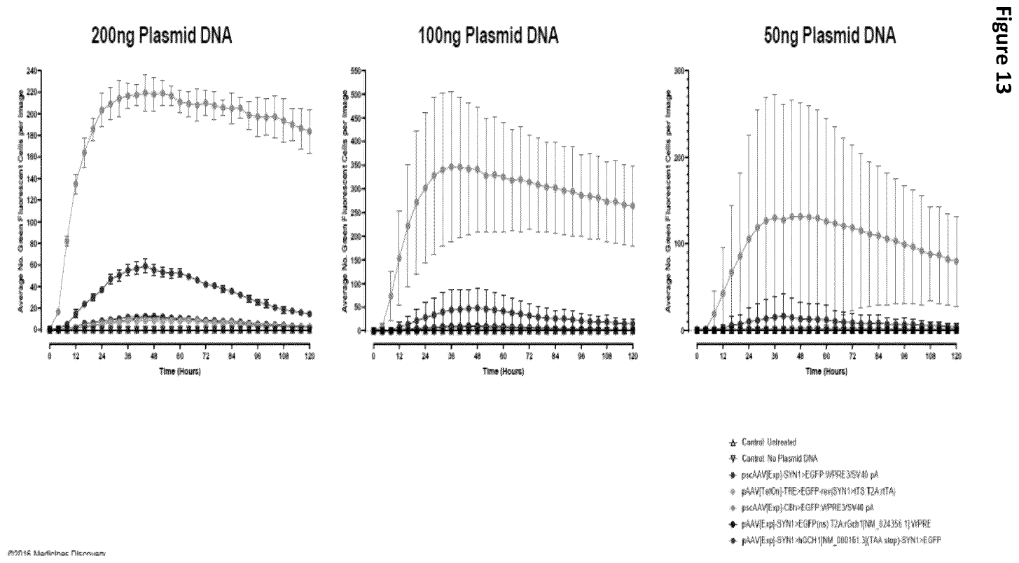

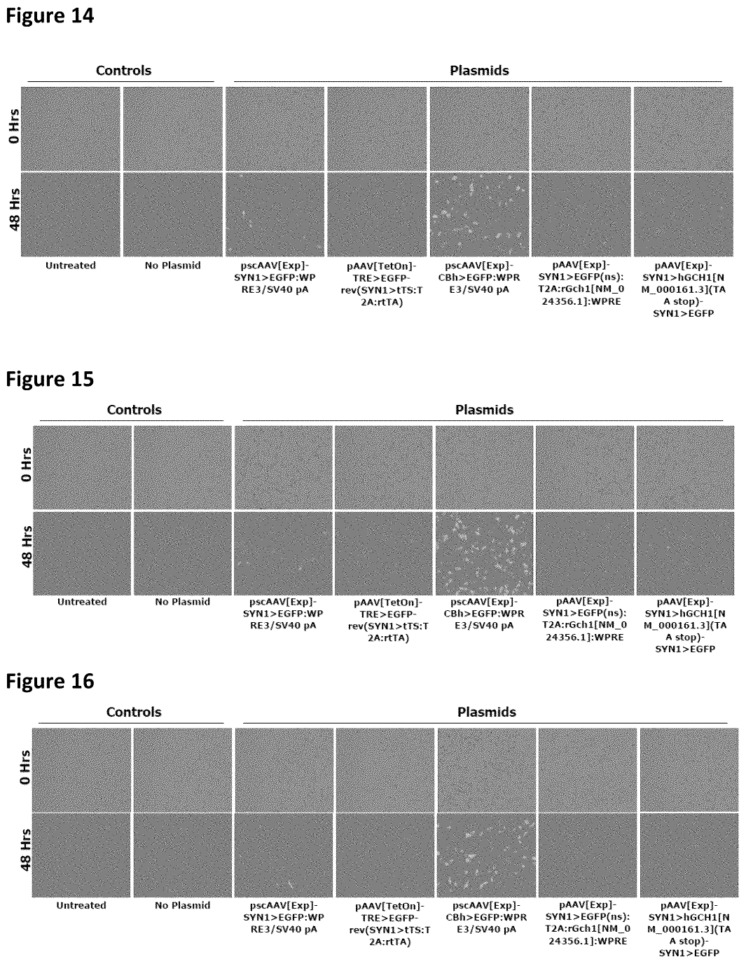

FIG. 14 shows the GFP expression in cells transfected with 200 ng of DNA after 48 hours.

FIG. 15 shows the GFP expression in cells transfected with 100 ng of DNA after 48 hours.

FIG. 16 shows the GFP expression in cells transfected with Song of DNA after 48 hours.

FIG. 17 shows a coronal section of MPTP-lesioned macaque brain stained with mouse anti-TH antibody (×2 magnification).

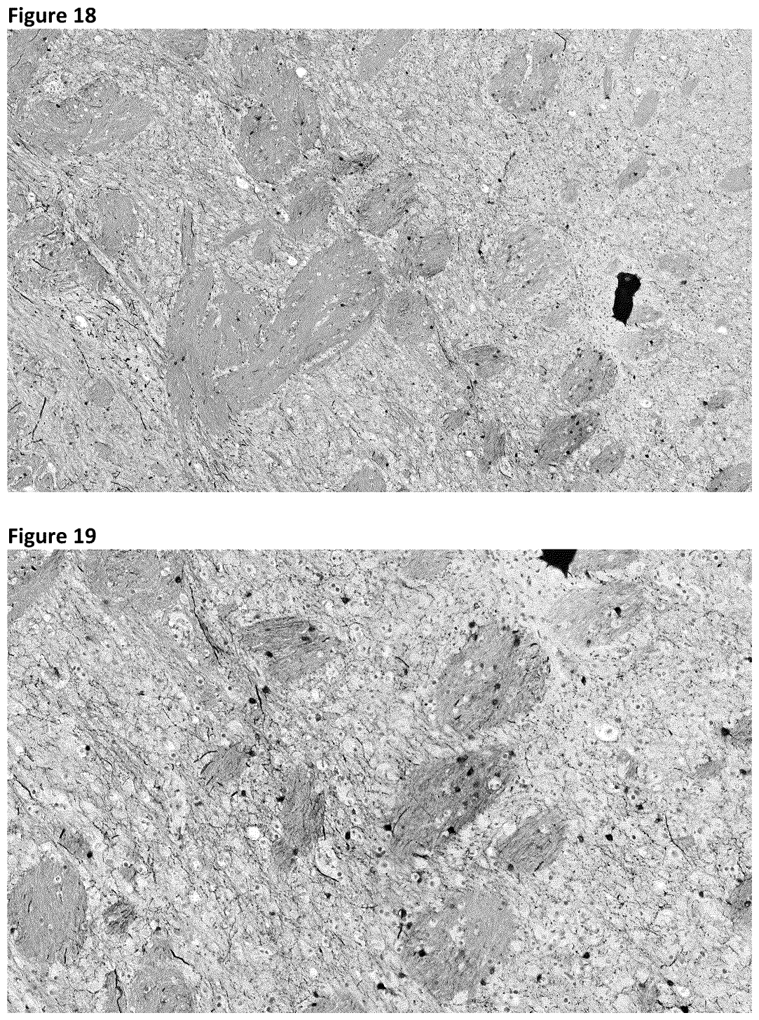

FIG. 18 shows a coronal section of MPTP-lesioned macaque brain stained with mouse anti-TH antibody (×10 magnification).

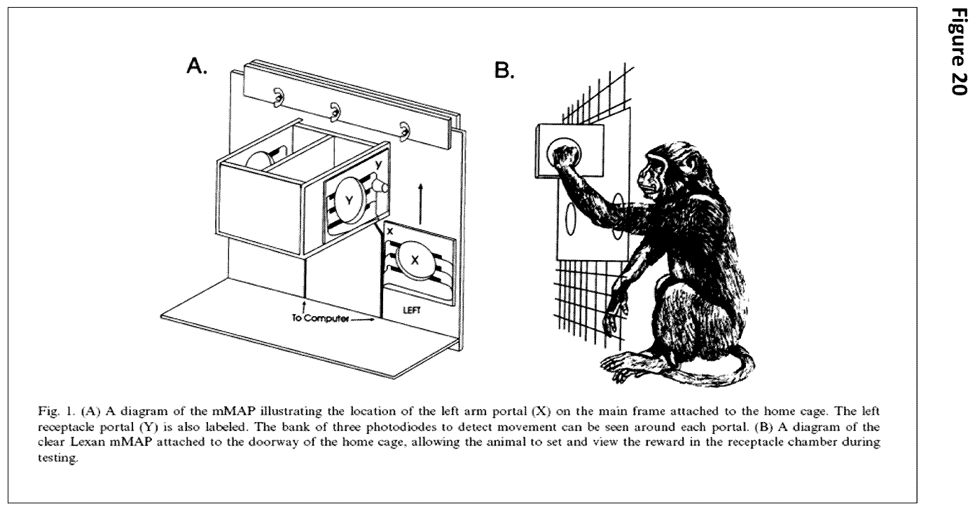

FIG. 19 shows a coronal section of MPTP-lesioned macaque brain stained with mouse anti-TH antibody (×20 magnification).

FIG. 20 shows a monkey movement analysis panel (mMAP; from Gash et al, 1999).

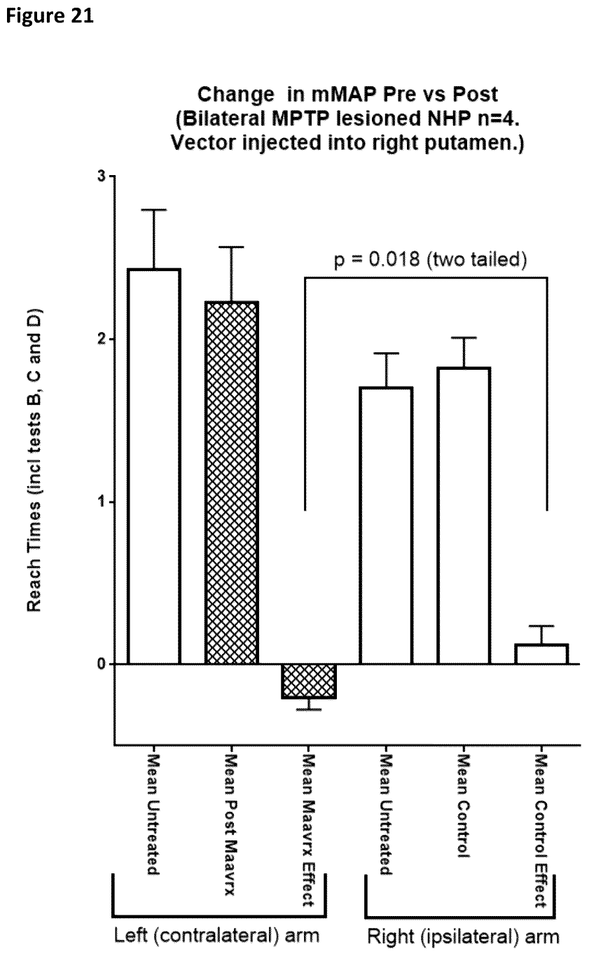

FIG. 21 shows results of monkey analysis panel experiments, i.e. the change in mMAP Pre vs Post treatment.

### EXAMPLES

### Background

The inventors set out to determine an optimum expression cassette (and vector harbouring the cassette) for in vivo expression of TH and GCH1. The objective of the study was to transfect SH-SY5Y (human neuronal cells) in vitro with five different plasmids at three different concentrations and analyse the expression of a reporter gene EGFP. In these constructs, the sequence for TH was substituted for EGFP to enable comparison of transduction efficiency by measurement of GFP fluorescence.

### Example 1

### Materials and Methods

The SH-SY5Y cells were obtained at P13 from Sigma-Aldrich, cultured in 10% FBS DMEM:F12 containing 2 mM L-Glutamine and banked at P15. A total of five transfection experiments were conducted to optimise the conditions. In brief, SH-SY5Y cells were plated directly from frozen in 96 well plates at 20,000 cells per well and cultured for 24 hr. The medium was removed and transfection was carried out using TransFast reagent in a 1:1 ratio of TransFast reagent to plasmid DNA in basal medium DMEM:F12 with no serum. Plasmids were assigned codes A-E as shown in Table 1 below. The transfection was carried out according to the instructions provided for the TransFast reagent using the equivalent of 0.5 ug, 0.75 ug and 1.0 ug per 24 well. Note that the Even et al publication used 0.75 ug. A total of 40 ul of TransFast plasmid mixture was added to each well of a 96 well plate and incubated for 1 hr. The calculation for the transfection reagent plasmid mixture is shown in Table 2 below. After 1 hr the TransFast plasmid mixture was removed and 200 ul of 10% FBS DMEM:F12 growth medium added and incubated for 2 days.

TABLE 1
Plasmid constructs

| | Plasmid | |
|---|---|---|
| Code | ID | Construct |
| A | VB200507- | pAAV[Exp]SYN1 > |
| | 3049fzk | hGCH1[NM_000161.3](TAAstop)-SYN1 > EGFP |
| B | VB200507- | pscAAV[Exp]CBh > EGFP:WPR E3/SV40 |
| | 1054ety | |
| C | VB200507- | pscAAV[Exp]SYN1 > EGFP:WP RE3/SV40 pA |
| | 1050huy | |
| D | VB200510- | pAAV[Exp]SYN1 > |
| | 1139ssd | EGFP(ns):T2A:rGch1[NM_024356.1]:WPRE |
| E | VB200519- | pAAV[TetOn]TRE > EGFP-rev(SYN1 > |
| | 1235vjq | tTS:T2A:rtTA) |

Constructs A and D were single-stranded AAV plasmids.

Constructs B and C were self-complementary AAV plasmids.

Referring to FIGS. 1-9, there are shown plasmid maps of different embodiments of the constructs described herein. FIG. 6 (SEQ ID No: 17) illustrates a plasmid map for one preferred embodiment of the first expression vector which encodes human truncated TH, and FIG. 7 (SEQ ID No: 18) shows the map for the second expression vector which encodes human GCH-1.

TABLE 2
Transfection calculations
Transfection Calculations

| | A | A1 | A2 | A3 | B | B1 | B2 | B3 | C | C1 | C2 | C3 |
|---|---|---|---|---|---|---|---|---|---|---|---|---|
| Four Conditions/24 well | 0 | 0.5 | 0.75 | 1 | 0 | 0.5 | 0.75 | 1 | 0 | 0.5 | 0.75 | 1 |
| Total DNA | 0 | 0.875 | 1.312 | 1.75 | 0 | 0.875 | 1.312 | 1.75 | 0 | 0.875 | 1.313 | 1.75 |
| Concentration DNA ug/ul | 0 | 2.677 | 2.677 | 2.677 | 0 | 0.196 | 0.196 | 0.196 | 0 | 0.342 | 0.342 | 0.342 |
| 1-1 DNA ul | 0.0 | 0.3 | 0.5 | 0.7 | 0.0 | 4.5 | 6.7 | 8.9 | 0.0 | 2.6 | 3.8 | 5.1 |
| TransFast | 0.0 | 2.6 | 3.9 | 5.3 | 0.0 | 2.6 | 3.9 | 5.3 | 0.0 | 2.6 | 3.9 | 5.3 |
| Medium | 0.0 | 347.0 | 345.6 | 344.1 | 0.0 | 342.9 | 339.4 | 335.8 | 350.0 | 344.8 | 342.2 | 339.6 |
| 1-1 Total | 350.0 | 350.0 | 350.0 | 350.0 | 350.0 | 350.0 | 350.0 | 350.0 | 350.0 | 350.0 | 350.0 | 350.0 |

| | D | D1 | D2 | D3 | E | E1 | E2 | E3 |
|---|---|---|---|---|---|---|---|---|
| Conditions/24 well | 0 | 0.5 | 0.75 | 1 | 0 | 0.5 | 0.75 | 1 |
| Total DNA | 0 | 0.875 | 1.3125 | 1.75 | 0 | 0.875 | 1.3125 | 1.75 |
| Concentration DNA ug/ul | 0 | 0.502 | 0.502 | 0.502 | 0 | 0.444 | 0.444 | 0.444 |
| 2-1 DNA ul | 0.0 | 1.7 | 2.6 | 3.5 | 0.0 | 2.0 | 3.0 | 3.9 |
| TransFast | 0.0 | 2.6 | 3.9 | 5.3 | 0.0 | 2.6 | 3.9 | 5.3 |
| Medium | 350.0 | 345.6 | 343.4 | 341.3 | 350.0 | 345.4 | 343.1 | 340.8 |
| 1-1 Total | 350.0 | 350.0 | 350.0 | 350.0 | 350.0 | 350.0 | 350.0 | 350.0 |

A total of 8 wells were transfected per condition. After two days incubation, the cells were gently washed 1×200 ul with warm PBS. Two wells per condition (top two rows of each 96 well plate) were fixed with 4% Paraformaldehyde containing 4% sucrose in PBS for 15 min. The wells were then washed 1×200 ul with PBS and stored in 100 ul PBS. Fluorescent plate analysis was conducted by scanning the top two rows of the 96 well plate with a Tecan plate reader using excitation filter 485 nm and emission filter 535 nm. Data is shown by subtracted the non-transfected well signals from the transfected wells (n=2).

All other wells were lysed with 1×SDS PAGE sample buffer (NuPage, Invitrogen) by adding 30 ul per well and pipetting up and down to lyse genomic DNA. The six wells for each condition were combined in one tube and boiled for 5 min before loading 50 ul of the cell lysate to a 4-12% NuPage Bis-Tris gel and separated by electrophoresis in MES running buffer. Western blotting of the gel was performed onto Nitrocellulose membrane in Novex Mini blot module using Bolt transfer buffer and 10% methanol. A prestained molecular weight marker was used to confirm transfer (EZ-Run prestained marker Fisher). In brief, the membrane was dried over night and blocked with 5% non-fat dried milk in PBS containing 0.05% Tween 20 (PBST). The membrane was probed for 1 hr with rabbit anti-egfp polyclonal antibody (Invitrogen CAB4211) at a dilution of 1:250 in PBST and then washed 3×5 min in PBST. The membrane was incubated in secondary anti-rabbit IgG HRP (Invitrogen cat #31460) at a dilution of 1:2500 for 1 hr and then washed 3×5 min in PBST. The western blots were then developed using a colorimetric DAB Substrate Kit from Thermo Fisher Cat #34002 for 15 min.

### Results

### First Data Set

As shown in FIG. 10A, the Western blot shows bands of the correct apparent molecular weight of EGFP of 37 KDa. Bands are clearly visible in plasmid construct B from 0.5 ug to 1.0 ug. Note that condition E1 was not run on the gel as there was only 10 wells per gel. A, B and C are control non-transfected cells. Levels of GFP expression were also determined using a fluorescence plate reader (FIG. 10B).

### Second Data Set

The second data set confirm the findings of the first data set, as shown in FIGS. 11A and B. Namely, the B construct (plasmid ID: VB200507-1054ety Construct: pscAAV[Exp]CBh>EGFP:WPR E3/SV40) shows the highest and dose dependent expression of EGFP and construct C (pscAAV[Exp]SYN1>EGFP:WP RE3/SV40 pA) showing a much lower but detectable expression of EGFP. Note in this experiment, the entire plate was scanned (n=8) as opposed to only the top two rows that were scanned in the first experiment (n=2). There was detectable expression in construct A but no expression was found in construct E incubating with 1 ug/ml doxycycline.

The Western blot analysis was loaded with a higher amount of sample than the first to increase the signal. In addition, TMB was used to develop the blot as it images much better. In the Western blot, expression is strongly detected in construct B and construct C, as shown in FIG. 11A. However, detection of expression was just above background in construct E incubated with 1 ug/ml doxycycline. It may be that a higher concentration of doxycycline is required but this would require a toxicity experiment as doxycycline is toxic >2 ug/ml in many cell lines. With the higher loading and TMB detection, expression of EGFP in construct A is just visible above background. No expression of EGFP is detected in construct D. (Con is Control, MW is Molecular Weight Marker).

### SUMMARY

The objective of the study was to transfect SH-SY5Y (human neuronal cells) in vitro with five different plasmids at three different concentrations and analyse the expression of a reporter gene EGFP to determine the best expression cassette for in vivo study and therapy. The SH-SY5Y were transfected at passage 15 (P15) with TransFast Reagent at 0.5 μg, 0.75 μg and 1 μg (equivalence to 24 well) in 96 well plates and analysed both qualitatively by Western Blot and quantitatively by fluorescence plate reader.

The results show that self-complementary plasmid ID: VB200507-1054ety Construct: pscAAV[Exp]CBh>EGFP:WPR E3/SV40 (construct B) had the highest expression of EGFP all five plasmids tested. Moreover, by both Western Blot and fluorescence plate reader, the expression of EGFP was dose dependant and increased from 0.5 μg to 1 μg. Expression of EGFP was observed at a lower level by only one other plasmid, i.e. self-complementary pscAAV[Exp]SYN1>EGFP:WP RE3/SV40 pA.

### Example 2—Pilot Study—Assess the Ability of AAV-TH/GCH1 on the Expression of Tyrosine Hydroxylase in a MPTP-Lesioned Macaque

### Study Outline

The study was a non-GLP study to assess the ability of AAV-TH/GCH1 to increase TH expression in the putamen following administration over 3 sites in the putamen.

MPTP is 1-methyl-4-phenyl-1,2,3,6-tetrahydropyridine and induces Parkinsonian syndrome. On Day 1 (D-7) of the study, a single female cynomolgus macaque previously lesioned by systemic MPTP and shows significant motor deficits that are reversible by L-DOPA, received a T1-weighted MRI for the purpose of imaging the anatomy from which to derive surgical targets.

On Do, the animal underwent stereotaxic surgery to administer AAV-TH/GCH1 over 3-sites within the right putamen. The left putamen received vehicle.

On D28, the animal was deeply sedated using pentobarbital and killed via exsanguination with heparinised saline.

Following rapid removal, the brain paraffin embedded before being placed ventral side up into a stainless steel brain matrix. The brain was blocked at 4 mm intervals to obtain slabs throughout the entire striatum. Slides from each brain slab were processed for TH-IHC and imaged on a 40× slide scanner.

All in-life components were conducted at Atuka's non-human primate facility at 1336 WuzhongAvenue, Suzhou, Jiangsu Province, PRC.

### Methods and Materials

### Constructs

The constructs of the invention, i.e. scAAV-TH/CGH1, were stored at −80° C.

### Vehicle

The vehicle used to formulate AAV-TH/CGH1 was PBS with 5% sorbitol.

### Animal Husbandry

The study was conducted under an approved IACUC. The macaque was obtained from Suzhou Xishan Zhongke Laboratory Animal Company (Xishan island, Jiangsu province, PRC). The animal was group housed with 2-3 animals per cage. The cage sizes exceeded UK, EU, NIH and CCAC minimum size recommendations, 152 (w)×136 (d)×192 (h) cm. The housing room was subject to a 12-hour light-dark cycle (lights on 7 a.m.), temperature 20-26° C. in a room containing only animals of the same sex. Fresh fruit, primate pellets and water was available ad libitum.

The animal was handled by technical staff and transferred from home caging to observation caging on a regular basis. The animal ID was identified via individually inscribed metal collar tags and also by a subcutaneously implanted transponder encoded with the animal ID (Plexx,model IPTT-300). The animal was weighed weekly throughout the duration of the study.

### Surgical Delivery of Viral Vectors

### MRI

For the purpose of calculating surgical coordinates for each animal and for each target, T1 weighted 3T MRI was performed. Animals were anesthetized with Zoletil (4-6 mg/kg, IM)/atropine 0.04 mg/kg, IM). Once sufficiently sedated, animals were mounted onto the surgical frame and coordinates recorded in order to place the animal back into the frame at the same orientation for surgery. Once in the frame the animal was placed into the MRI scanner and 0.3 mm thick horizontal slices was obtained throughout the brain. Images were stored on external hard drives and submitted to Osirix imaging software for viewing and derivation of surgical targets. Part of this process included a qualitative inspection of the brain to note any abnormal neuroanatomical presentations (e.g. tumor, abnormal asymmetry).

### Surgery (Do)

Stereotaxic injection of the AAV vector was performed under isoflurane anaesthesia in sterile conditions. All cranial injections were performed with the animal in a stereotaxic head holder. Precise stereotaxic coordinates for all surgeries were calculated prior to surgery from each individual animal MRI scan. Following sterile preparation, incisions were made in the scalpover the target area and skin, muscle and fascia retracted to expose the cranial surface. Single large bilateral burr holes were made over the target areas. A small incision was then be made in the dura above the desired injection sites and the tip of a Hamilton syringe (26G) was lowered to the desired site for microinjection. Injections containing the AAV were made at a speed of 1.0 μL/min and a volume of 15 μL each into 3 sites of each hemisphere of the putamen (AC+1, AC-2 and AC-5 mm). At each site, 2 deposits were made at 2 different depths. The needle was maintained in place for an additional 5 min after each injection before being withdrawn very slowly and moved to the next site. Following injection, the exposed dura was covered with Gelfoam and the incision was closed with an interrupted 6-0 monofilament suture. Animals received antibiotic treatment just prior to surgery (ampicillin, 160 mg/kg, IM) and 2×/day thereafter for 3 days.

Postoperatively, for pain control, animals received meloxicam for 5 days (1/day, 0.1 mg/kg, PO). The animals were carefully monitored during the post operative period (see details below).

### Treatments

The experiment contained 1 animal. Details can be found in the table below,

| | Surgery, | | |
|---|---|---|---|
| | right | Surgery, left | |
| Treatment | putamen | putamen | |
| group | (Day 0) | (Day 0) | Necropsy |
| 1 | AAV-TH/GCH1 | vehicle | Day 28 |

### Brain Tissue Preparation

On D28, the animal was removed from their home cage, administered NSD1015 (dose and route to be determined) 30 minutes prior to necropsy. The animal was then deeply sedated by overdose with sodium pentobarbital (50 mg/kg, IV).

The thoracic cavity was quickly opened and a 14G catheter was inserted into the ascendingaorta, the descending aorta was clamped behind the heart and an incision made into the right atrium to allow efflux of returning venous blood. Using a perfusion pump approximately 200 ml of ice-cold heparinised 0.9% saline (10 000 IU/L) was perfused at a rate of 100 ml/min. The brains were then removed and placed ventral side up, into a pre-chilled ice-cold brain matrix. Brain slabs of 4 mm thickness were made spanning the entire striatum and from one of the slabs, single punches of putamen were taken from both hemispheres and stored at −80° C. Slabs of tissue were then immersed in 10% formalin for 48 hrs before being subjected to paraffin embedding. Tissues were then sectioned onto glass slides for immunohistochemistry.

### Postmortem Measures

### Tyrosine Hydroxylase Histology

From 5 um thick paraffin-embedded sections from both hemispheres, mounted on glass slides, slides were deparaffinized and rehydrated as follows: twice in 100% Xylene (5 minutes each), twice in 100% Ethanol (3 minutes each), once in 95% Ethanol (1 minute), once in 70% Ethanol (1 minute), twice in double distilled water (3 minutes each). Heat induced antigen retrieval was performed by incubating slides in Citrate Buffer for 20 minutes, followed by cooling at room temperature for approximately 20 minutes. Following three washes in PBS, endogenous peroxidase was quenched in 0.6% hydrogen peroxide solution and background staining was inhibited in a 10% normal goat serum/2% bovine serum albumin 0.1% Triton PBS solution. Tissue was then incubated with primary antibody overnight: Mouse anti-TH antibody (1:50, ThermoFisher. No. 185). After three washes in PBS, sections were sequentially incubated in biotinylated goat anti-mouse IgG (1:500, Jackson Immuno Research Laboratories, West Grove, PA, Cat. No. 111-065-144) for 1 hour at room temperature. Following 3 washes in PBS, sections were incubated with the Elite avidin-biotin complex (ABC kit, Vector, Burlingame, Ca, Cat. No. PK-6101) for 30 minutes. Immunostaining was visualized following a 15 minute reaction with 3, 3 diaminobenzidine (DAB kit, Vector, Burlingame, Ca, Cat. No. SK-4100). Sections were allowed to dry, dehydrated through graded alcohols (70%, 95%, 100%), cleared in xylenes and coverslipped with DEPEX mounting medium (Electron Microscopy Sciences, Hatfield, PA, Cat. No. 13514). Each slide was digitally scanned at 40× magnification (Aperio XT, Leica) and images will be provided.

### Results

Referring to FIGS. 17-19, there are shown coronal sections of MPTP-lesioned macaque brain stained with Mouse anti-TH antibody (1:50, ThermoFisher. No. 185). TH-expressing fibres and cell bodies are stained brown. Expression of TH is demonstrated in the area where a combination of scAAV-htTH and scAAV-GCH1 was injected.

### DISCUSSION

This study was conducted as previous attempts by others to transduce sufficient TH expression to be detected by immunohistochemistry in the MPTP macaque model of PD by transduction with a bicistronic vector failed, i.e. Cederfjill, E. et al. Continuous DOPA synthesis from a single AAV: dosing and efficacy in models of Parkinson's disease. Sci Rep-uk 3, (2013), in which the authors state “The reason for the lack of transgenic TH expression by histology and lack of DOPA and dopamine production by microdialysis remains unclear at this time. However, this problem requires a solution prior to the initiation of clinical trials utilizing this approach.”.

In contrast, the study described herein demonstrates that the claimed invention resulted in sufficient expression of TH in the MPTP-lesioned macaque putamen to be easily detected using equivalent immunohistochemistry detection.

### Example 3—Assessment of the Effects of 1:1 Combination of scAAV5-htTH and scAAV5-GCH1 on Behaviour in the MPTP-Lesioned Macaque

### Objective

The purpose of this study was to assess the ability of a 1:1 mixture of scAAV-TH and scAAV-GCH1 administered unilaterally to the putamen to improve contralateral motor performance on a reaching task in animals with an existing motor disability from MPTP exposure.

### Animal Welfare

The study was conducted according to CCAC guidelines and under IACUC-approved Animal Use Protocols (AUPs).

### Study Outline

The animals selected for the study had been previously lesioned via systemic administration of MPTP and show stable bilateral motor deficits that are sensitive to L-DOPA therapy. The behavioural tasks examined included a reaching task (monkey movement assessment panel, mMAP) and, independently, observation cage measures of general locomotor activity (assessed via passive infra-red activity monitors during a 2-hour period and by use of Actical over a 24 hour period).

Behavioural assessments (mMAP and activity) was made twice weekly prior to surgery for 3 weeks (for a total of 6 observations) and twice weekly, every two weeks post-surgery for a period of 3 months post-surgery (for a total of 12 observations).

On Day D-20 of the Study, all animals will received a T1-weighted MRI for the purpose of imaging the anatomy from which to derive surgical targets.

On D1, the animals will underwent stereotaxic surgery to administer 1:1 mixture of AAV-TH and AAV-GCH1 over 3-sites within the right putamen.

### Methods and Materials

The scAAV-TH and scAAV-GCH1 were stored at −80 and were brought to room temperature before co-infusion in PBS with 5% sorbitol.

The macaques were obtained from Suzhou Xishan Zhongke Laboratory Animal Company (Xishan island, Jiangsu province, PRC). Animals were acclimatised to the experimental setting. The animals will had been rendered parkinsonian by once daily subcutaneous injection of 0.2 mg/kg MPTP, administered for 8-12 days, until the first appearance of parkinsonian symptoms. After this time, a parkinsonian syndrome reached a moderate to marked level, over approximately 30 days, and stabilized. Additional administrations of MPTP were given to some animals to titrate to similar degrees of parkinsonism in individuals across the group. The macaques were allowed to recover for a minimum of further 30 days until their parkinsonism was demonstrated as being stable.

Sixty days after commencing MPTP administration, L-DOPA (25 mg/kg) was administered orally twice daily for at least two months. L-DOPA is given with the decarboxylase inhibitor benserazide (as Madopar™). This treatment leads to the development of motor fluctuations, including dyskinesia. Once selected for the current study animals received no further regular L-DOPA administration.

For the purpose of calculating surgical coordinates for each animal and for each target, T1 weighted 3T MRI was performed. Animals were anesthetized with Zoletil (4-6 mg/kg, IM/atropine 0.04 mg/kg, IM). Once sufficiently sedated, animals were mounted onto the surgical frame and coordinates recorded in order to place the animal back into the frame at the same orientation for surgery. Once in the frame the animal was placed into the MRI scanner and 0.3 mm thick horizontal slices will be obtained throughout the brain. Images were stored on external hard drives and submitted to Osirix imaging software for viewing and derivation of surgical targets.

Stereotaxic injection of the AAV vectors was performed under isoflurane anesthesia in sterile conditions. Injections containing the mixture of two AAV vectors will be made by an infusion pump at a speed of 2.0 uL/min (Pump 11 Elite Nanomite Programmable Syringe Pump, Harvard Apparatus). The needle was based on the construction described in WIPO Patent number WO2006/042090 AI (Kankiewicz and Sommer). Two stacked deposits, each of 15 μL of AAV mixture will be equally spaced along three tracks. The three tracks will be positioned pre-commissural, commissural and post-commissural in the putamen. Thus, a total of 90 μL of the AAV mixture will be deposited in the right putamen of each animal. The needle tip was positioned at the target site for the proximal deposit (1 minute wait time) followed by cannula advancement and infusion at the distal deposit (5 minute wait time).

Animals received antibiotic treatment just prior to surgery (ampicillin, 160 mg/kg, IM) and 2×/day thereafter for 3 days. Postoperatively, for pain control, animals will receive meloxicam for 5 days (1/day, 0.1 mg/kg, PO).

| Surgery | Behaviour | Number |
|---|---|---|
| Right | Right and left arm mMAP and | of |
| Putamen | activity counts | animals |
| 1:1 | mMAP reach times, twice per week at | 4 |
| scAAV- | weeks −3, −2, and −1 prior to surgery and | |
| TH:scAAV- | twice every 2 weeks post-surgery for 3 months | |
| GCH1 | (all tests without L-DOPA/benserazide) | |
| mixture | | |

### Behaviour

The primary endpoint of the study was fine motor function of the right and left upper limb assessed, in the animal's home cage, using a reaching task. Prior to commencing the study, animals were been trained to retrieve a positive reinforcer (candy), using the right and left hands from the monkey movement analysis panel as illustrated in FIG. 20 (mMAP; essentially equivalent to that described by Gash D M, et al., J Neurosci Methods. 1999; 89:111-7. KoW Ltd, Mississauga, ON, Canada).

The trial began when the experimenter places the candy (Lifesaver) in the mMAP located outside the animal's home cage within reach of the animal. The time taken for the animal to retrieve the candy (“Retrieval Time”) and return its arm to within the home cage, with a maximum cut off of 45 seconds, was assessed post-hoc by analysis of video recordings by an observer blinded to the treatment given, The ability of the animal to perform at three ascending levels of difficulty was assessed whereby the Lifesaver treat is either placed on the floor of the mMAP receptacle (Level B), on a straight pin within the receptacle (C) or, and most challengingly, on a curved hook (D) within the receptacle. The MAP tests were videoed in such a way that the resulting digital video file will allow a third-party rater blinded to treatment allocation or side of surgery to independently repeat the measurement of retrieval time.

### Results

Referring to FIG. 21, there is shown the change in mMAP Pre vs Post treatment. As can be seen, treatment resulted in a significantly (p=0.018) greater reduction in the reach time as a percent of baseline reach time in the contralateral arm compared to the ipsilateral arm. The reduction in reach time for the contralateral arm is consistent with increased L-DOPA production within the lesioned hemisphere. (The observed increase in reach time in the ipsilateral arm is unexplained and could be due to chance or could reflect some reversal of the increased dopamine D2 receptors known to occur in untreated MPTP lesioned animals. The precent reduction in reach time would approach significance (p=0.052) even if no change was assumed in the ipsilateral arm.)

### OVERALL CONCLUSIONS

It is known that Parkinson's disease symptoms arise due to a lack have the production of dopamine in the striatum of the brain. Three enzymes are necessary to produce dopamine, namely tyrosine hydroxylase [TH], GTP cyclohydrolase 1 [GCH] and amino acid decarboxylase [AADC]. Multiple attempts, including preclinical and clinical studies, have attempted to provide symptomatic relief of Parkinson's disease by using gene therapy to restore production of dopamine in the striatum by introducing one, two, or three of the genes to produce these enzymes. To date, however, none of these attempts has resulted in an optimal solution.

The invention described herein embodies co-administration off preferably two self-complementary AAV viral vectors encoding transduction of tyrosine hydroxylase and GTP cyclohydrolase 1 into intraparenchymal injection into the striatum. In contrast to the only published study using a transducing just TH and GCH1 (without AADC) into the putamen of the MPTP NHP model of PD the invention resulted in expression of TH detectable by immunohistochemistry. Despite many previous published studies in which one or both genes have been injected into the striatum of animal models or patients to reduce the motor symptoms of Parkinson's disease, the composition described herein has never been described previously. Importantly, the novel co-administration of two monocistronic self-complementary AAV vectors has not been viewed as an obvious strategy. The enhanced efficacy and reduced cost of goods resulting from the invention are surprising and clinically important.

The inventor has found no reference to the co-administration of two self-complementary AAV vectors as a treatment for Parkinson's disease in the published literature are any other publicly available source.

The efficacy and economic advantages of the invention are advantageous and surprising for the following reasons:
(i) Two previously published approaches have coadministered TH and GCH in combination with AADC the resultant improvement in the motor symptoms of animal models of Parkinson's disease. While both these approaches have incorporated coadministration of TH and GCH neither has demonstrated that the use of TH and GCH in the absence of coadministration with AADC is effective. This is important because separate studies in non-human primates and in humans have emphasized the importance off loss of AADC in the evolution of clinical Parkinson's disease and have demonstrated significant symptomatic improvement in Parkinson's disease following intraparenchymal injection into the striatum of AADC alone. Based on the publications in which all three genes have been coadministered either as three monocistronic AAV vectors or as a single tricistronic lenti vector, it is not possible to predict the efficacy of TH and GCH administered in the absence of AADC.
(ii) A series of preclinical studies demonstrated that coadministration of single-stranded monocistronic AAV vectors carrying the transgenes for TH and GCH resulted in an improved motor performance when administered directly into the striatum of 6 hydroxy dopamine lesioned rats. However, this approach was not investigated further in larger animals or man, the researchers reasoning that it “has a number of limitations, as transduction of efficacy and transgene expression is difficult to predict by in vitro assays clinical production would become very troublesome.” Secondly, although at the global scale the expression pattern over the two genes might look similar, the number of copies of the two vectors in an individual cell might vary dramatically, thus resulting in varying grade of DOPA synthesis. In addition, the effect might be aggravated with many cells receiving none or only one of the genes and therefore displaying limited DOPA synthesis if any. Furthermore, co-administration of different vectors carrying the same serotype was postulated to risk competition between the vectors for the same binding site on the target cell. Finally, and importantly, it was also reasoned that the cost of goods for a therapy requiring two individual AAV vectors would be significantly higher than a therapy requiring a single bicistronic vector. For these reasons, investigators switched to developing a bicistronic vector delivering both TH and GCH.
(iii) A series of studies subsequently demonstrated that administration of TH and GCH in a bicistronic single stranded [i.e., not self-complementary] AAV vector improved Parkinson disease motor symptoms in rat. Although this approach produced complete reversal of motor symptoms in the six hydroxy dopamine rat model of Parkinson's disease, it produced only modest improvement when scaled up to the non-human primate model. This was consistent with low barely discernible expression (assessed by immunohistochemistry) of TH in the striatum of treated non-human primates. As these findings were observed at the highest feasible dose of the bicistronic vector, development of this product was terminated and not advanced into clinical trials. Indeed, in a resulting publication, the authors state “The reason for the lack of transgenic TH expression by histology and lack of DOPA and dopamine production by microdialysis remains unclear at this time. However, this problem requires a solution prior to the initiation of clinical trials utilizing this approach.” Since these findings, no further studies implying that dual use of only TH and GCH have been published and the approach seems to have been abandoned as not sufficiently efficacious.
(iv) Co-administration of two self-complementary AAV vectors for this indication is novel and results in markedly enhanced transduction of tyrosine hydroxylase to an extent that could not have been predicted. Clear evidence of efficacy in MPTP lesioned non-human primates has been demonstrated following co-administration of the two self-complementary monocistronic vectors.
(v) The invention uses a CBh promoter that has never been applied to vectors intended to treat Parkinson's disease. The CBh promoter offers the following advantages over promoters used in previous vectors intended to treat Parkinson's disease, i.e. (1) its short length enables accommodation of the promoter trans gene combination within a self-complementary AAV construct. (2) It is less prone to silencing then the CMV promoter widely used in previous monocistronic constructs. (3) Its lack of neuronal specificity enables transduction of astrocytes and glia increasing the potential for additional production of DOPA by these cells within the striatum (4), the CBh promoter contains both a truncated chicken beta-actin intron and a minute virus of mouse (MVM) intron, which, together, act as a spacer, thereby increasing gene expression.
(vi) Importantly, the increase in efficacy observed along human primates with the invention is such that the dose of the two individual self-complementary vectors is more than 50% lower than then the [moderately] effective dose of the previously researched bicistronic factor. This surprising finding has significant potential beneficial impact on the cost of goods for the resulting therapeutic product.

1-39. (canceled)

40. A composition comprising first and second expression vectors, wherein the first expression vector comprises a promoter operably linked to a self-complementary coding sequence, which encodes tyrosine hydroxylase (TH), and the second expression vector comprises a promoter operably linked to a coding sequence, which encodes GTP cyclohydrolase 1 (GCH1).

41. The composition according to claim 40, wherein:
(i) the first expression vector and/or the second expression vector is a naked DNA vector;
(ii) the first expression vector and/or the second expression vector is an AAV vector;
(iii) the second expression vector is a single-stranded AAV (ssAAV) vector; and/or
(iv) the second expression vector is a self-complementary AAV vector.

42. The composition according to claim 40, wherein:
(i) the first expression vector is self-complementary AAV vector, and the second expression vector is a ssAAV or naked DNA vector;
(ii) the first and/or second expression vector is derived from AAV-1, AAV-2, AAV-3A, AAV-3B, AAV-4, AAV-5, AAV-6, AAV-7, AAV-8, AAV-9, AAV-10, and/or AAV-11;
(iii) the first and/or second expression vector is derived from AAV1, AAV5, or AAV9, and more preferably, AAV5; and/or
(iv) the composition does not comprise a vector, which encodes aromatic amino acid decarboxylase (AADC).

43. The composition according to claim 40, wherein:
(i) the coding sequence encoding TH comprises a nucleotide sequence substantially as set out in SEQ ID No: 1 or 21, or a fragment or variant thereof, or wherein the coding sequence encoding TH comprises a nucleotide sequence encoding an amino acid sequence substantially as set out in SEQ ID No: 2 or 22, or a fragment or variant thereof;
(ii) the coding sequence encoding TH comprises a nucleotide sequence encoding truncated TH lacking the regulatory domain of TH, optionally
wherein the coding sequence encoding TH comprises a nucleotide sequence substantially as set out in SEQ ID No: 3 or 23, or a fragment or variant thereof, or wherein the coding sequence encoding TH comprises a nucleotide sequence encoding an amino acid sequence substantially as set out in SEQ ID No: 4 or 24, or a fragment or variant thereof; and/or
(iii) the coding sequence encoding GCH1 comprises a nucleotide sequence substantially as set out in SEQ ID No: 6, or a fragment or variant thereof, or wherein the coding sequence encoding GCH1 comprises a nucleotide sequence encoding an amino acid sequence substantially as set out in SEQ ID No: 7, or a fragment or variant thereof.

44. The composition according to claim 40, wherein:
(i) the promoter in the first and/or second expression vector is one that permits high expression in a subject's neurons, or in the subject's glial cells, or in the subject's neurons and glial cells, or in the subject's neurons and ependymal cells lining the cerebral ventricles, or in the subject's neurons and glial cells and ependymal cells;
(ii) the promoter in the first and/or second expression vector is the CBh promoter, or a fragment or variant thereof, optionally wherein the promoter in the first and second vectors comprises the CBh promoter, optionally
wherein the promoter sequence in the first and/or second expression vector comprises a nucleotide sequence substantially as set out in SEQ ID No: 8, or a fragment or variant thereof; and/or
(iii) the promoter in the first and/or second expression vector is a human synapsin promoter, or the chicken beta actin promoter with a cytomegalovirus enhancer (CB7), or a Tetracycline-responsive element (TRE) promoter, and optionally is not the CMV promoter, or the CMV enhancer/promoter, optionally
wherein the promoter comprises a nucleotide sequence substantially as set out in SEQ ID No: 9, 10, 11, or 25, or a fragment or variant thereof.

45. The composition according to claim 40, wherein the first expression vector and/or the second expression vector comprises an intron disposed between its promoter and the nucleotide encoding TH1 or GCH1, respectively, optionally wherein
(i) the intron is at least 25, 50, 75, or 100 nucleotides in length;
(ii) the intron is at least 125, 150, 175, or 200 nucleotides in length; or
(iii) the intron is at least 225, 250, 275, or 300 nucleotides in length.

46. The composition according to claim 45, wherein the intron is selected from a group of introns consisting of: the human growth hormone (hGH) intron; the beta-actin intron; the minute virus of mouse (MVM) intron; the SV40 intron; and the EF-1 alpha intron, optionally wherein the intron is the MVM intron, preferably wherein the intron comprises a nucleotide sequence substantially as set out in SEQ ID No: 27, or a fragment or variant thereof.

47. The composition according to claim 45, wherein:
(i) the first and/or second expression vector comprises a SYN1 promoter followed by an intron, which is either the MVM intron (SEQ ID No: 27) or the human growth hormone (hGH) intron (SEQ ID No: 26);
(ii) the first and/or second expression vector comprises a chicken beta actin promoter with a cytomegalovirus enhancer (CB7) followed by an intron, which is either the MVM intron or the human growth hormone (hGH) intron;
(iii) the first and/or second expression vector comprises a Tetracycline-responsive element (TRE) promoter followed by an intron, which is either the MVM intron or the human growth hormone (hGH) intron; or
(iv) the first and/or second expression vector comprises a CMV promoter followed by an intron, which is either the MVM intron or the human growth hormone (hGH) intron.

48. The composition according to claim 40, wherein second expression vector further comprises a nucleotide sequence encoding Woodchuck Hepatitis Virus Post-transcriptional Regulatory Element (WPRE), optionally wherein the WPRE coding sequence is disposed 3′ of GCH1 coding sequence, and/or wherein the WPRE comprises a nucleic acid sequence substantially as set out in SEQ ID No: 12 or 13, or a fragment or variant thereof.

49. The composition according to claim 40, wherein the first and/or second expression vector comprises a nucleotide sequence encoding a polyA tail, wherein:
(i) the polyA tail comprises the simian virus SV40 polyA tail, optionally comprising a nucleic acid sequence substantially as set out in SEQ ID No: 14, or a fragment or variant thereof; and/or
(ii) the polyA tail comprises the bovine growth hormone (BGH) poly A tail, optionally comprising a nucleic acid sequence substantially as set out in SEQ ID No: 15, or a fragment or variant thereof.

50. The composition according to claim 40, wherein:
(i) the first expression vector and/or the second expression vector comprises a nucleotide sequence encoding a 3′ untranslated region (3′ UTR), optionally wherein the first expression vector comprises a 3′ UTR coding sequence comprising a nucleic acid sequence substantially as set out in SEQ ID No: 28, or a fragment or variant thereof;
(ii) the first and/or second expression vector comprises a left and/or a right Inverted Terminal Repeat sequences (ITRs), optionally wherein the first and/or second expression vector comprises one Inverted Terminal Repeat (ITR) sequence and one modified ITR sequence in which the terminal resolution site is deleted, optionally comprising an ITR comprising a nucleic acid sequence substantially as set out in SEQ ID No: 16, or a fragment or variant thereof;
(iii) the first expression vector comprises a nucleic acid sequence substantially as set out in SEQ ID No: 17, or a fragment or variant thereof; and/or
(iv) the second expression vector comprises a nucleic acid sequence substantially as set out in SEQ ID No: 18, or a fragment or variant thereof.

51. The composition according to claim 40, wherein the composition comprises:—
(i) a first self-complementary adeno-associated virus (scAAV) vector comprising a promoter operably linked to a coding sequence, which encodes tyrosine hydroxylase (TH), optionally truncated TH lacking the regulatory domain; and
(ii) a second self-complementary adeno-associated virus (scAAV) vector comprising a promoter operably linked a coding sequence, which encodes GTP cyclohydrolase 1 (GCH1).

52. A pharmaceutical composition comprising the composition according to claim 40, and a pharmaceutically acceptable vehicle.

53. A method of treating, preventing, or ameliorating Parkinson's disease, DOPA responsive dystonia, vascular parkinsonism, side effects associated with L-DOPA treatment for Parkinson's disease, L-DOPA induced dyskinesia, Segawa syndrome, or genetic dopamine receptor abnormalities in a subject, the method comprising administering, to a subject in need of such treatment, a therapeutically effective amount of the composition according to claim 1, or the pharmaceutical composition according to claim 52.

54. The method of claim 53, wherein the composition or the pharmaceutical composition are administered into the blood stream, the cerebrospinal fluid, a nerve, or the brain; and/or wherein the composition or the pharmaceutical composition are administered into the striatum, the putamen or caudate nucleus, dopaminergic neurons of the pars compacta region in the substantia nigra.

55. The method of claim 53, wherein the dose of the composition delivered is 300 μl to 20,000 μl, 300 μl to 10,000 μl, 300 μl to 5,000 μl, 300 μl to 4500 μl, 400 μl to 4000 μl, 500 μl to 3500 μl, 600 μl to 3000 μl, 700 μl to 2500 μl, 750 μl to 2000 μl, 800 μl to 1500 μl, 850 μl to 1000 μl, or approximately 900 μl; and/or
wherein if administered as a mixture of AAV vectors, the titre of each AAV is 1E8 to 5E14, 1E9 to 1E14, 1E10 to 5E13, 1E11 to 1E13, 1E12 to 8E12, 4E12 to 6E12, or roughly 5E12 genome copies per ml (GC/ml).

56. The method of claim 53, wherein if administered as a mixture of naked DNA plasmid vectors, the dose of each DNA plasmid vector is 50, 100, 200, 300, 400, 500, 600, 700, 800, 900, 1000, 1250, 1500, 1750 or 2000 micrograms (μg) per brain hemisphere.

57. A method of treating, preventing, or ameliorating Parkinson's disease, DOPA responsive dystonia, vascular parkinsonism, side effects associated with L-DOPA treatment for Parkinson's disease, L-DOPA induced dyskinesia, Segawa syndrome, or genetic dopamine receptor abnormalities in a subject, the method comprising administering, to a subject in need of such treatment, a therapeutically effective amount of a first expression vector comprising a promoter operably linked to a self-complementary coding sequence, which encodes tyrosine hydroxylase (TH), and a second expression vector comprising a promoter operably linked to a coding sequence, which encodes GTP cyclohydrolase 1 (GCH1).

58. The method of claim 57, wherein the first and second expression vectors are as defined in claim 1.

59. A kit of parts comprising the first and second expression vectors as defined in claim 40, and optionally, instructions for use, optionally
wherein the kit comprises a first container in which the first expression vector is contained, and a second container in which the second expression vector is contained, optionally wherein the first and/or second container is a vial, syringe, Eppendorf, or the like.
